(19) United States
(12) Patent Application Publication
(10) Pub. No.: US 2007/0059702 A1
(43) Pub. Date: Mar. 15, 2007

(54) Disease related protein network

(76) Inventors: Erich Wanker, Berlin (DE); Hans Lehrach, Berlin (DE); Heike Gohler, Berlin (DE); Martin Strodicke, Berlin (DE); Ulrich Stelzl, Berlin (DE); Maciej Lalowski, Berlin (DE)

Correspondence Address: Louis M Heidelberger;Reed Smith, 2500 One Liberty Place, 1650 Market Street, Philadelphia, PA, 19103, US

(21) Appl. No.: 10/561,669

(22) PCT Filed: Jun. 18, 2004
(86) PCT No.: PCT/EP04/06617

§ 371 Date: Dec. 20, 2005

(30) Foreign Application Priority Data
Jun. 20, 2003 (EP) 03013957.0

(51) Int. Cl. C40B 40/08; C40B 30/06; C12Q 1/68; C07H 21/04; C12P 21/06; C07K 14/705

(52) U.S. Cl. 435/6; 435/69.1; 435/320.1; 435/325; 530/350; 536/23.5

(57) ABSTRACT

The present invention relates to a method for generating a network of direct and indirect interaction partners of a disease-related (poly)peptide comprising the steps of (a) contacting a selection of (poly)peptides suspected to contain one or several of said direct or indirect interaction partners with said disease-related (poly)peptides and optionally with known direct or indirect interaction partners of said disease-related (poly)peptide under conditions that allow the interaction between interaction partners to occur; (b) detecting (poly)peptides that interact with said disease-related (poly)peptide or with said known direct or indirect interaction partners of said disease-related (poly)peptide; (c) contacting (poly)peptides detected in step (b) with a selection of (poly)peptides suspected to contain one or several (poly)peptides interacting with said (poly)peptides detected in step (b) under conditions that allow the interaction between interaction partners to occur; (d) detecting proteins that interact with said (poly)peptides detected in step (b); (e) contacting said disease-related (poly)peptide and optionally said known direct or indirect interaction partners of said disease-related (poly)peptide, said (poly)peptides detected in steps (b) and (d) and a selection of proteins suspected to contain one or several (poly)peptides interacting with any of the afore mentioned (poly)peptides under conditions that allow the interaction between interaction partners to occur; (f) detecting (poly)peptides that interact with said disease-related (poly)peptide and optionally said known direct or indirect interaction partners of said disease-related (poly)peptide or with said (poly)peptides identified in step (b) or (d); and (g) generating a (poly)peptide-(poly)peptide interaction network of said disease-related (poly)peptide and optionally said known direct or indirect interaction partners of said disease-related (poly)peptide and said (poly)peptides identified in steps (b), (d) and (f). Moreover, the present invention relates to a protein complex comprising at least two proteins and to methods for identifying compounds interfering with an interaction of said proteins. Finally, the present invention relates to a pharmaceutical composition and to the use of compounds identified by the present invention for the preparation of a pharmaceutical composition for the treatment of Huntington's disease.

The present invention relates to a method for generating a network of direct and indirect interaction partners of a disease-related (poly)peptide comprising the steps of (a) contacting a selection of (poly)peptides suspected to contain one or several of said direct or indirect interaction partners with said disease-related (poly)peptides and optionally with known direct or indirect interaction partners of said disease-related (poly)peptide under conditions that allow the interaction between interaction partners to occur; (b) detecting (poly)peptides that interact with said disease-related (poly)peptide or with said known direct or indirect interaction partners of said disease-related (poly)peptide; (c) contacting (poly)peptides detected in step (b) with a selection of (poly)peptides suspected to contain one or several (poly)peptides interacting with said (poly)peptides detected in step (b) under conditions that allow the interaction between interaction partners to occur; (d) detecting proteins that interact with said (poly)peptides detected in step (b); (e) contacting said disease-related (poly)peptide and optionally said known direct or indirect interaction partners of said disease-related (poly)peptide, said (poly)peptides detected in steps (b) and (d) and a selection of proteins suspected to contain one or several (poly)peptides interacting with any of the afore mentioned (poly)peptides under conditions that allow the interaction between interaction partners to occur; (f) detecting (poly)peptides that interact with said disease-related (poly)peptide and optionally said known direct or indirect interaction partners of said disease-related (poly)peptide or with said (poly)peptides identified in step (b) or (d); and (g) generating a (poly)peptide—(poly)peptide interaction network of said disease-related (poly)peptide and optionally said known direct or indirect interaction partners of said disease-related (poly)peptide and said (poly)peptides identified in steps (b), (d) and (f). Moreover, the present invention relates to a protein complex comprising at least two proteins and to methods for identifying compounds interfering with an interaction of said proteins. Finally, the present invention relates to a pharmaceutical composition and to the use of compounds identified by the present invention for the preparation of a pharmaceutical composition for the treatment of Huntington's disease.

Several documents are cited throughout the text of this specification. The disclosure content of the documents cited herein (including any manufacture's specifications, instructions, etc.) is herewith incorporated by reference. The present invention is based on scientific experiments which have been performed on biological specimen derived from diseased patients. Patients have given their consent to use the specimen for the study which is disclosed in the present invention. In case of deceased patients, the consent has been given by a relative.

With the identification of >35.000 genes in the human genome the challenge arises to assign biological function to all proteins and to link these proteins to physiological pathways and disease processes. Since protein-protein interactions play a role in most events in a cell, clues to the function of an unknown protein can be obtained by investigating its interaction with other proteins whose function are already known. Thus, if the function of one protein is known, the function of the binding parners can be infered (deduced). This allows the researcher to assign a biological function to uncharacterized proteins by identifying protein-protein interactions. For example, several so far uncharacterized proteins in Caenorhabditis elegans were identified in a yeast two-hybrid screen for eukaryotic 26S proteasome interacting proteins and thereby could be linked to the ubiquitin-proteasome proteolytic pathway (Vidal et al., 2001). Elucidation of protein-protein interactions is particularly desired when it comes to the generation of new drugs. For many diseases, the available drug portfolio is insufficient or inappropriate to provide a cure or to prevent onset of the disease. One such disease is Huntington's disease.

Huntington's disease (HD) is a neurodegenerative disorder caused by an expanded polyglutamine (polyQ) tract in the multidomain protein huntingtin (htt). The elongated polyQ sequence is believed to confer a toxic gain of function to htt. It leads to htt aggregation primarily in neurons of the striatum and cortex and subsequently to the appearance of the disease phenotype. However, there is experimental evidence that loss of htt function may also contribute to HD pathogenesis. Since huntingtin aggregation correlates with disease progression, it is crucial to develop methods for identifying factors that promote or inhibit aggregation of huntingtin.

Previously, a number of single interaction partners of huntingtin had been reported. In light of these reports, it is tempting to speculate that huntingtin is bound into a larger network of interacting partners, many of which might be capable of modulating huntingtin's activity and function by direct or indirect interaction. It is likely that an aberrant interaction of huntingtin with some of the members of said network will impair huntingtin's normal function. Moreover, this interaction might also be relevant for the conformation of huntingtin or for its solubility or state of aggregation. Interfering with the direct or indirect interactions of the protein-protein interaction network will provide an excellent basis for therapeutic intervention as it will allow to modulate huntingtin's activity or state of aggregation or both. The state of the art so far did not provide compounds capable of reducing or suppressing huntingtin aggregation since the factors promoting or suppressing huntingtin aggregation were not known.

Thus, the technical problem underlying the present invention was to provide novel approaches for identifying direct or indirect interaction partners of disease-related proteins, which must be seen as new targets for drug development. The solution to this technical problem is achieved by providing the embodiments characterized in the claims.

Accordingly, the present invention relates to a method for generating a network of direct and indirect interaction partners of a disease-related (poly)peptide comprising the steps of (a) contacting a selection of (poly)peptides suspected to contain one or several of said direct or indirect interaction partners with said disease-related (poly)peptides and optionally with known direct or indirect interaction partners of said disease-related (poly)peptide under conditions that allow the interaction between interaction partners to occur; (b) detecting (poly)peptides that interact with said disease-related (poly)peptide or with said known direct or indirect interaction partners of said disease-related (poly)peptide; (c) contacting (poly)peptides detected in step (b) with a selection of (poly)peptides suspected to contain one or several (poly)peptides interacting with said (poly)peptides detected in step (b) under conditions that allow the interaction between interaction partners to occur; (d) detecting proteins that interact with said (poly)peptides detected in step (b); (e) contacting said disease-related (poly)peptide and optionally said known direct or indirect interaction partners of said disease-related (poly)peptide, said (poly)peptides detected in steps (b) and (d) and a selection of proteins suspected to contain one or several (poly)peptides interacting with any of the afore mentioned (poly)peptides under conditions that allow the interaction between interaction partners to occur; (f) detecting (poly)peptides that interact with said disease-related (poly)peptide and optionally said known direct or indirect interaction partners of said disease-related (poly)peptide or with said (poly)peptides identified in step (b) or (d); and (g) generating a (poly)peptide-(poly)peptide interaction network of said disease-related (poly)peptide and optionally said known direct or indirect interaction partners of said disease-related (poly)peptide and said (poly)peptides identified in steps (b), (d) and (f).

In accordance with the present invention, the term “direct and indirect interaction partners” relates to (poly)peptides that either directly interact with the disease-related (poly)peptide (direct interaction) or that interact via a protein binding to/interacting with said disease-related (poly)peptide. In the letter case, there is no direct contact between the direct interaction partner and the disease-related protein. Rather, a further protein forms a “bridge” between these two proteins.

The term “known direct or indirect interaction partners” refers to the fact that for certain disease-related (poly)peptides, such interaction partners are known in the art. If such interaction partners are known in the art, it is advantageous to include them into the method of the invention. If no such interactions partners are known in the art, then the network may be generated starting solely from the known disease-related (poly)peptide.

The term “conditions that allow the interaction between interaction partners to occur” relates to conditions that would, as a rule, resemble physiological conditions. Conditions that allow protein actions are well known in the art and, can be taken, for example from Golemis, E. A. Ed., Protein-Protein Interactions, Cold Spring Harbor Laboratory Press, 2002.

The term “suspected to contain one or more of said direct or indirect interaction partners” relates to the fact that normally, a selection of (poly)peptides would be employed where the person skilled in the art would expect that interaction partners are present. Examples of such selections of (poly)peptides are libraries of human origin such as cDNA libraries or genomic libraries.

The term “detecting proteins” refers to the fact that the (poly)peptides interacting with the “bait” (poly)peptides are identified within the selection of (poly)peptides. A further characterization or isolation of the “prey” (poly)peptides at this stage may be advantageous but is not necessary. The term “detecting (poly)peptides” preferably also comprises characterizing said (poly)peptides or the nucleic acid molecules encoding said (poly)peptides. The skilled person knows that this can be done by a number of techniques, some of which are described for example in Sambrook et al., “Molecular Cloning, A Laboratory Manual”; CSH Press, Cold Spring Harbor, 1989 or Higgins and Hames (eds.). For example, the nucleotide sequence may be determined by DNA Sequencing, including PCR-Sequencing (see for example Mullis K, Faloona F, Scharf S, Saiki R, Horn G, Erlich H., Cold Spring Harb Symp Quant Biol. 1986; 51 Pt 1:263-73). Alternatively, the amino acid sequence of said (poly)peptide may be determined. The skilled artesian knows various methods for sequencing proteins which include the method of Edman degradation, which is a preferred method of the present invention of determining the amino acid sequence of a protein. However, the amino acid sequence of a protein or (poly)peptide can also be reliably determined by methods such as for example Maldi-Tof, optionally in combination with the method of Edman degradation. The interaction partner may be identified either as fusion with a DNA binding domain or as fusion with an activation domain. Preferably, if an interaction partner has been identified as a fusion molecule comprising a DNA binding domain, the interaction partner is cloned into a vector allowing the expression of the interaction partner as a fusion with an activation domain. Consequently, protein interaction can be tested in the context the DNA activation or the DNA binding domain.

In accordance with the present invention, the first round of detecting (poly)peptides that interact with the “bait” (poly)peptides recited in step (a) wherein the detected (poly)peptides be considered as “prey” (poly)peptides is followed by the second round of detecting further interacting (poly)peptides wherein the former “prey” (poly)peptides are now used as “bait” (poly)peptides. In certain preferred embodiments of the present invention such as in a two-hybrid detection system, a re-cloning of the former “prey” (poly)peptides into vectors that are suitable for expressing “bait” (poly)peptides may be desired.

Accordingly, the invention describes a novel strategy to identify protein-protein interaction networks for human disease proteins. This strategy was applied to detect pair-wise protein-protein interactions for Huntington's disease and is useful for other hereditary diseases as well. Several human hereditary diseases are summarized in table 5.

A crucial step of the method of the invention is step (e). Here, the disease-related (poly)peptide and optionally said known direct or indirect interaction partners of said disease-related (poly)peptide are contacted under appropriate conditions, preferably at the same time, with both the (poly)peptides identified in steps (b) and (d) and further with a selection of (poly)peptides suspected to contain further interaction partners. Alternatively, the various baits, preys and further selection partners are added one after another, so that the final pool contains all baits and preys so far identified, in addition to the further selection partners. In other terms, in this step of the method of the invention, all “baits” and all “preys” are pooled and, additionally, further potential interaction partners are added. In this way, surprisingly the number of directed or indirect interactions partners of the previously identified “baits” and “preys” could significantly be enhanced. It is to be understood that various preys identified in one detection step may interact with each other and not only with the baits that were employed for the identification. For example, if a collection of baits detects prays “a” and “b”, the invention does not exclude that “a” also interacts with “b”. The same holds true mutatis mutandis for the baits used in accordance with the present invention. Wherever possible, baits and preys are exchangeable in the sense that bait (poly)peptides may be used as preys and vice versa. In a given case, however, the skilled person has to determine whether or not this exchange is possible on the basis of unfavourable site effects and limitations of the applied scientific approach. This can be done by the skilled person without undue burden by applying standard techniques known in the art.

It is further preferred in accordance with the present invention that the interaction of proteins is a specific interaction, such as a specific binding. This means that the (poly)peptide being an interaction partner with a further (poly)peptide only or essentially only interacts with the interaction site(s) involved with this interaction partner. This does not exclude, of course, that further interaction sites of said (poly)peptide interact with further interaction partners, wherein in the corresponding interaction is preferably also specific. The concept also embraces that, if a (poly)peptide has several identical interaction sites, which in nature bind to different interaction partners, these different interaction partners are also bound by the (poly)peptide in the method of the present invention.

In other terms, at least in the case of huntingtin, the number of interaction partners found in step (e) was enhanced in an exponential rather than in a linear fashion.

The term “(poly)peptide” refers alternatively to peptide or to (poly)peptides. Peptides conventionally are covalently linked amino acids of up to 30 residues, whereas polypeptides (also referred to as “proteins”) comprise 31 and more amino acid residues.

The term “huntingtin” refers to a protein with the data bank accession number P42858 which is referenced for the purpose of the present invention as “wild-type huntingtin protein”. However, the term “huntingtin” also comprises proteins encoded by the nucleic acid sequence deposited under accession number L12392 or to proteins encoded by nucleic acid molecules which hybridize to the nucleic acid molecule of L12392 under stringent conditions of hybridization. The present invention relates to all variants of the huntingtin protein. In particular, relevant for the present invention are those variants of huntingtin which comprise a polyglutamine tract (polyQ tract) or an elongated polyQ tract. A polyQ tract consists of two or more glutamines within the huntingtin protein. The insertion of additional glutamine codons will result in huntingtin proteins with, for example 2, 51, 75 or 100 added glutamines in comparison to the sequence deposited under accession number P42858. In fact, the person skilled in the art knows that the huntingtin protein may have a glutamine tract with any random number of glutamines in the range of 1 to 200 added glutamines. All these proteins are comprised by the present invention.

The term “hybridizes under stringent conditions”, as used in the description of the present invention, is well known to the skilled artisian and corresponds to conditions of high stringency. Appropriate stringent hybridization conditions for each sequence may be established by a person skilled in the art on well-known parameters such as temperature, composition of the nucleic acid molecules, salt conditions etc.; see, for example, Sambrook et al., “Molecular Cloning, A Laboratory Manual”; CSH Press, Cold Spring Harbor, 1989 or Higgins and Hames (eds.), “Nucleic acid hybridization, a practical approach”, IRL Press, Oxford 1985, see in particular the chapter “Hybridization Strategy” by Britten & Davidson, 3 to 15. Stringent hybridization conditions are, for example, conditions comprising overnight incubation at 42° C. in a solution comprising: 50% formamide, 5×SSC (750 mM NaCl, 75 mM trisodium citrate), 50 mM sodium phosphate (pH 7.6), 5× Denhardt's solution, 10% dextran sulfate, and 20 micrograms/ml denatured, sheared salmon sperm DNA, followed by washing the filters in 0.1×SSC at about 650. Other stringent hybridization conditions are for example 0.2×SSC (0.03 M NaCl, 0.003M Natriumcitrat, pH 7) bei 65° C. In addition, to achieve even lower stringency, washes performed following stringent hybridization can be done at higher salt concentrations (e.g. 5×SSC). Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The skilled person knows that the presence of additional codons in the nucleic acid sequence of huntingtin might significantly reduce the capability of this nucleic acid molecule to hybridize to the nucleic acid molecule deposited under L12392 and referenced as wild-type huntingtin protein. Nevertheless, such proteins shall still be comprised by the present invention. In fact, computer programs such as the computer program Bestfit (Wisconsin Sequence Analysis Package, Version 8 for Unix, Genetics Computer Group, University Research Park, 575 Science Drive, Madison, Wis. 53711) or blast, capable of calculating homologies between two nucleic acid sequences, efficiently recognize nucleotide insertions and allow for an adjustment of gaps created by these insertions. The term “huntingtin” as used in the present invention, also includes those molecules of huntingtin, which have a homology of more than 95% to wild-type huntingtin when analyzed with a program like bestfit under conditions not weighing gaps created by polyQ tracts (gap penalty=0).

The term “contacting” means bringing into contact so that two or more proteins or (poly)peptides can interact with each other, preferably under physiological conditions. The terms “interacting” or “binding” refer to a transient or permanent contact between two proteins or (poly)peptides. Preferably, the (poly)peptide or protein is provided by expression from a nucleic acid molecule, more preferably from a cDNA molecule within a cDNA library. Alternatively, said nucleic acid molecule is a genomic nucleic acid molecule of a genomic DNA library, or a nucleic acid molecule from a synthetic DNA or RNA library. Preferably, the nucleic acid molecule encoding the disease-related protein or its interaction partner is obtainable from nerve cells, brain tissue human adrenal gland, human bladder, human bone, human brain, human colon, human dorsal root ganglion, human heart, human HeLa cells, human kidney, human liver, human lung, human mammary gland, human ovary, human pancreas, human placenta, human prostate, human retina, human salivary gland, human sceletal muscle, human small intestine, human smooth muscle, human spinal cord, human spleen, human stomach, human testis, human thymus, human thyroid, human tonsil, human trachea, human uterus, human cell line HEP G2, human cell line MDA 435, human fetal brain, human fetal heart, human fetal kidney, human fetal liver, human fetal spleen, human fetal thymus, human breast tumor, human cervix tumor, human colon tumor, human kidney tumor, human lung tumor, human ovary tumor, human stomach tumor, human brain tumor and/or human uterus tumor.

The term “disease-related protein” refers to a protein known to be the causative agent of a disease or known to be involved in onset or progression of a disease. Preferably, said disease is CHOREA HUNTINGTON or the disease-related protein is huntingtin. More preferably, the disease-related protein is selected from table 6 and/or 7. The term “conditions that allow the interaction between interaction partners” means conditions that are similar to physiological conditions. Preferably, said conditions are physiological conditions.

The term “selection of (poly)peptides” refers to a library of (poly)peptides, which comprises the above-mentioned libraries, but also includes libraries such as phage display libraries. Preferably, the (poly)peptide is provided by expression from a nucleic acid molecule. Preferably, the protein or (poly)peptide expressed by said nucleic acid molecule is a (poly)peptide comprising a DNA binding domain (DBD) (in this case the fusion protein is termed “bait”) or (b) a (poly)peptide comprising an activation domain capable of interacting with a transcription factor or an RNA polymerase and capable of activating transcription of a reporter or indicator gene (in this case the fusion protein is called “prey”). As used here, the terms “reporter gene” and “indicator gene” are to be understood as synonyms. It is important to note that one of the interaction partners will always comprise the amino acid sequence of a protein or (poly)peptide translated from said nucleic acid molecule while the other interaction partner will comprise the amino acid sequence of a protein or protein fragment. Preferably, a bait used for a method of the present invention is selected from the proteins listed in table 6 and/or 7. If, for example, the proteins encoded by the nucleic acid molecules contain a DNA binding domain fused in frame, the fusion protein can bind to the DNA recognition sequence of the DNA binding domain. Interaction of said fusion protein with a second fusion protein containing an activation domain can induce transcription of a nearby indicator gene. The indicator gene may encode a selection marker such as a protein that confers resistance to an antibiotic including ampicillin, kanamycin, chloramphenicol, tetracyclin, hygromycin, neomycin or methotrexate. Further examples of antibiotics are Penicillins: Ampicillin HCl, Ampicillin Na, Amoxycillin Na, Carbenicillin disodium, Penicillin G, Cephalosporins, Cefotaxim Na, Cefalexin HCl, Vancomycin, Cycloserine. Other examples include Bacteriostatic Inhibitors such as: Chloramphenicol, Erythromycin, Lincomycin, Tetracyclin, Spectinomycin sulfate, Clindamycin HCl, Chlortetracycline HCl. Additional examples are proteins that allow selection with Bacteriosidal inhibitors such as those affecting protein synthesis irreversibly causing cell death. Aminoglycosides can be inactivated by enzymes such as NPT II which phosphorylates 3′-OH present on kanamycin, thus inactivating this antibiotic. Some aminoglycoside modifying enzymes acetylate the compound and block their entry in to the cell. Gentamycin, Hygromycin B, Kanamycin, Neomycin, Streptomycin, G418, Tobramycin Nucleic Acid Metabolism Inhibitors, Rifampicin, Mitomycin C, Nalidixic acid, Doxorubicin HCl, 5-Flurouracil, 6-Mercaptopurine, Antimetabolites, Miconazole, Trimethoprim, Methotrexate, Metronidazole, Sulfametoxazole. Alternatively, said indicator gene may encode a protein such as lacZ, GFP or luciferase, the expression of which can be monitored by detection of a specific color. Other proteins commonly used as indicator proteins are beta-galactosidase, beta-glucuronidase, green fluorescent protein (GFP), autofluorescent proteins, including blue fluorescent protein (BFP), glutathione-5-transferase (GST), luciferase, horseradish peroxidase (HRP), and chloramphenicol acetyltransferase (CAT). In general, however, the selection in the yeast two hybrid-system is based on a deficiency of the yeast strain to produce specific amino acids. The skilled person knows that any amino acid deficiency can be used for this selection strategy.

Preferably said preys and baits are expressed from two separate expression vectors contained in one host cell. The nucleic acid molecule encoding the preys and baits can be introduced into the host cell, for example, by transformation, transfection, transduction or microinjection which are common techniques known to the person skilled in the art and which require no additional explanation. In addition, the nucleic acid molecule contains a chromosomal or episomal nucleic acid sequence encoding the above-mentioned indicator protein. The expression of said indicator protein is under control of a recognition sequence which serves as a binding site for the bait protein. The nucleic acid molecule may be fused either to a DNA binding domain or to an activation domain. Co-expression of only those bait- and prey fusion proteins which are capable of interacting will induce the expression of one of the above-identified indicator proteins and thus allow the identification a nucleic acid molecule encoding a protein capable of interacting with huntingtin or an interaction or binding partner of huntingtin. The skilled person knows this system as the yeast two hybrid system. The yeast two hybrid system, which uses a bait protein-prey protein combination to induce transcription of the reporter gene, is a preferred method to identify proteins capable of interacting with huntingtin or with a direct or indirect interaction or binding partner of huntingtin. See for example Fields and Song, Nature 340:245 (1989) or Uetz et al., 2000 Nature 403(6770): 623-7. This is a useful way of determining protein-protein interactions. Another preferred method uses the yeast three hybrid system, as described in U.S. Pat. No. 5,928,868. Preferably, steps (a) to (d) of the method for generating a network of direct and indirect interaction partners comprise the yeast two hybrid system. Preferably, steps (e) and (f) of the method for generating a network of direct and indirect interaction partners comprise yeast interaction mating. Preferably, said “interaction mating” comprises the interaction of all interaction partners identified in steps (a) to (d). Also preferred is that the interaction partners identified in steps (a) to (d) interact as prey and bait proteins, so that all prey proteins are contacted with all bait proteins. Using the array mating system, each bait is tested individually for interaction with every prey in the array. Alternatively, steps (e) and (f) of the method for generating a network of direct and indirect interaction partners comprise testing all interaction partners identified in steps (a) to (d) in interaction assays such as biacore or coimmunoprecipitation. When performing such an assay, it is preferred that the interaction partners are tested as prey and/or bait fusion proteins or contain no fused (poly)peptides. Preferably, all interaction partners are contacted in the biacore or coimmunoprecipitation assay by themselves and by all other remaining interaction partners identified in steps (a) to (d).

The method for generating a network of direct and indirect interaction partners of a disease related protein or (poly)peptide has proven to be an effective tool for unveiling the protein-protein interactions (PPI) of preferably monogenic diseases. This is exemplified by the analysis of the disease related protein of Chorea Huntington, the analysis of which has demonstrated that the method of the present invention will be useful in an approach to identify potential drugs in the treatment of CHOREA HUNTINGTON. Moreover, this method will also be effective in unveiling the protein-protein interactions of other disease related proteins and in identifying novel targets for treatment of these diseases. Using a preferred combination of library and matrix yeast two-hybrid screens, based on the methods of the present invention, a highly connected network was generated among 70 proteins involved in 117 protein-protein interactions, 99 of which had not been described previously. As progression of Huntington's disease (HD) appears to be linked to huntingtin aggregation, a set of network proteins was tested for their potential to modulate this process. By using the methods of the present invention, it was discovered that the GTPase activating protein GIT1 strongly promotes huntingtin aggregation in vivo. GIT1 also localises to huntingtin aggregates in brains of transgenic mice and HD patients. Therefore, a combination of the methods of the present invention has proven to provide effective means for the identification of potential targets for therapeutic intervention. GIT1 is a selected example of a modulator interaction partner of huntingtin. The other proteins in the network of interaction partners disclosed by the present invention are further modulator interaction partners of huntingtin.

Preferably, the interaction mating comprises using an array maiting system. In general, for this screen, MATα yeast cultures are transformed with plasmids encoding prey proteins and arrayed on a microtiter plate for interaction mating with individual MATa strains expressing bait proteins. Using this test system, each bait can be tested individually for interaction with every prey in the array. Diploid yeast clones, formed by maiting on YPD plates and expressing both, bait and prey proteins, are selected on agar SDII plates, and further transferred for example by a spotting robot on SDIV plates to select for protein-protein interactions. In a more preferred embodiment of the method, plasmids encoding bait and prey proteins are transformed into strains L40 ccua and L40 cca, respectively. L40 cca clones are arrayed on microtitre plates and mixed with a single L40 ccua clone for interaction mating. These cells are transferred, preferably by a robot onto YPD medium plates and, after incubation for 20 h to 28 h at approximately 30° C., for selection of the cells, were transferred onto SDII medium plates, where mating takes place, for additional 60 h to 80 h at approximately 30° C. For two-hybrid selection diploid cells are transferred onto SDIV medium plates with and without nylon or nitrocellulose membranes and incubated for approximately 5 days at about 30° C. The nylon or nitrocellulose membranes are subjected to the β-GAL assay. Positive clones can be verified by cotransformation assays using plasmids encoding respective bait and prey proteins. Other preferred methods for studying protein-protein interactions according to the present invention are colocalization, coimmunoprecipitation, screening of protein or (poly)peptide arrays, library screens, in vivo and in vitro binding experiments using different tags such as HIS6, TAP or FLAG.

In a preferred embodiment of the present invention's method for generating a network of direct and indirect interaction partners of a disease related protein or (poly)peptide, plasmids encoding bait proteins are transformed into a strain such as L40 ccua, tested for the absence of reporter gene activity and co-transformed with a human fetal brain cDNA library. Independent transformants are plated onto minimal medium lacking tryptophan, leucine, histidine and uracil (SDIV medium) and incubated at about 30° C. for 5 to 10 days. Clones are transferred into microtitre plates, optionally using a picking robot, and grown over night in liquid minimal medium lacking tryptophan and leucine (SDII medium). Subsequently, the clones are spotted onto nylon or nitrocellulose membranes placed on SDIV medium plates. After incubation for about 4 days membranes are subjected to a β-galactosidase (β-GAL) assay. Plasmids are prepared from positive clones and characterised, for example by restriction analyses and sequencing. For retransformation assays plasmids encoding bait and prey proteins are cotransformed in the yeast strain L40 ccua and plated onto SDIV medium.

The term “generating a protein-protein interaction (PPI) network” means listing the interactions of all proteins interacting or binding directly or indirectly interacting the disease related (poly)peptide or protein. Preferably, this can be done by displaying the information in a matrix or a network representation. In a more preferred embodiment of the present invention's method, the protein-protein interaction network is generated by using Pivot 1.0 (Prof. Ron Shamir, Prof. Yossi Shilo, Nir Orlev; Tel Aviv University (TAU); Dep. of computer science; Ramat Aviv; Tel Aviv 69978; Israel).

In a preferred embodiment of the invention, interactions are detected by using the yeast two-hybrid system, MALDI-TOF MS or electro spray MS. Preferably, yeast strains such as strains L40 ccua and L40 cca, are transformed with an expression selected from the group consisting of pBTM116, pBTM117, pBTM117c, pACT2, pAS2-1, pGADIO, pGAD424, pGAD425, pGAD426, pGAD427, pGAD428.

In another preferred embodiment of the present invention's method for generating a network of direct and indirect interaction partners of a disease-related polypeptide, the method contains after step (d) the additional steps of isolating a nucleic acid molecule with homology to said nucleic acid molecule expressing the encoded protein and testing it for its activity as a modulator of huntingtin, wherein said nucleic acid molecule is DNA, RNA, cDNA, or genomic DNA. Said testing can be done in several different assays. Preferably, the testing is performed in a co-immunoprecipitation assay or an affinity chromatography-based technique. Generally, co-immunoprecipitation is performed by purifying an interacting protein complex with a single antibody specific for one protein in the protein complex and by detecting the proteins in the protein complex. The step of detection can involve the use of additional antibodies directed against proteins suspected of being trapped in the purified protein complex. Alternatively, at least one protein in the protein complex is fused to a tag sequence with affinity to a compound fixed to a solid matrix. By contacting the solid matrix with said tagged protein, further proteins binding to said protein can be purified and binding can be detected. GST or HA are preferred tags in accordance with the present invention.

In a preferred embodiment of the present invention's method, said contacting step (e) is effected in an interaction mating two hybrid approach.

In another preferred embodiment of the present invention's method, said method comprises after step (d) and before step (e) the steps of: (d′) contacting (poly)peptides detected in step (d) with a selection of (poly)peptides suspected to contain one or several (poly)peptides interacting with said (poly)peptides detected in step (d) under conditions that allow the interaction between interaction partners to occur; and (d″) detecting proteins that interact with said (poly)peptides detected in step (d′).

This preferred embodiment of the invention, an additional step of identifying further interaction partners is carried out prior to the contacting of all “baits” and “preys” in one pool (step (e)). Optionally, further steps of selecting interaction partners in analogy to steps (d′) and (d″) may be infected prior to the pooling/interaction step.

Diseases of particular interest for which interrelationships of disease-related proteins may be analyzed in accordance with the invention are provided in Table 5.

In yet another preferred embodiment of the present invention's method, said disease related protein is a protein suspected of being a causative agent of a hereditary (see Table 5), such as a monogenic disease.

In another preferred embodiment of the present invention's method, said disease related protein is huntingtin and said interaction partners are the interaction partners as shown in table 6,7 and/or 9

In another preferred embodiment of the present invention's method, said method comprises the step of determining the nucleotide sequence of a nucleic acid molecule encoding a direct or indirect interaction partner of the disease related protein.

In another preferred embodiment of the present invention's method, said selections of proteins are translated from a nucleic acid library.

In, another preferred embodiment of the present invention's method, said selection of proteins in step (a) and/or (c) and/or (d′) and/or (e) is the same selection or a selection from the same source. In another preferred embodiment of the present invention's method, said selection of proteins in step (a) and/or (c) and/or (d′) and/or (e) is a different selection or a selection from a different source.

Preferably, said source is selected from nerve cells, brain tissue, human adrenal gland, human bladder, human bone, human brain, human colon, human dorsal root ganglion, human heart, human HeLa cells, human kidney, human liver, human lung, human mammary gland, human ovary, human pancreas, human placenta, human prostate, human retina, human salivary gland, human sceletal muscle, human small intestine, human smooth muscle, human spinal cord, human spleen, human stomach, human testis, human thymus, human thyroid, human tonsil, human trachea, human uterus, human cell line HEP G2, human cell line MDA 435, human fetal brain, human fetal heart, human fetal kidney, human fetal liver, human fetal spleen, human fetal thymus, human breast tumor, human cervix tumor, human colon tumor, human kidney tumor, human lung tumor, human ovary tumor, human stomach tumor, human brain tumor and/or human uterus tumor.

In another preferred embodiment of the present invention's method, said method is performed by contacting the proteins on an array. Preferably, said array is an array allowing to detect protein-protein interaction by the principle of a biacore detector.

In another preferred embodiment of the present invention's method, said interactions are detected by using the yeast two-hybrid system. Preferably, said inteactions detected by using MALDI-TOF, MS, electro spray MS or biacore.

In another preferred embodiment of the present invention's method, said method contains after step of (b), (d), (d″) or (f) the additional steps of isolating a nucleic acid molecule with homology to said cDNA expressing the encoded protein and testing it for its activity as a modulator of huntingtin, wherein said nucleic acid molecule is DNA, or RNA, and preferably cDNA, or genomic or synthetic DNA, or mRNA.

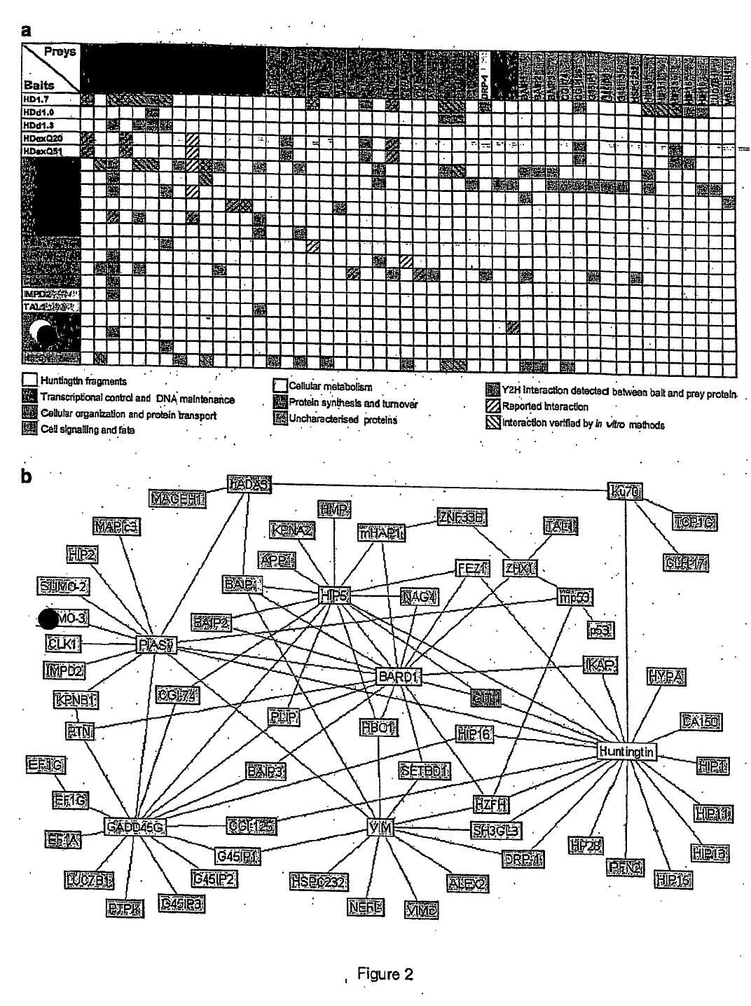

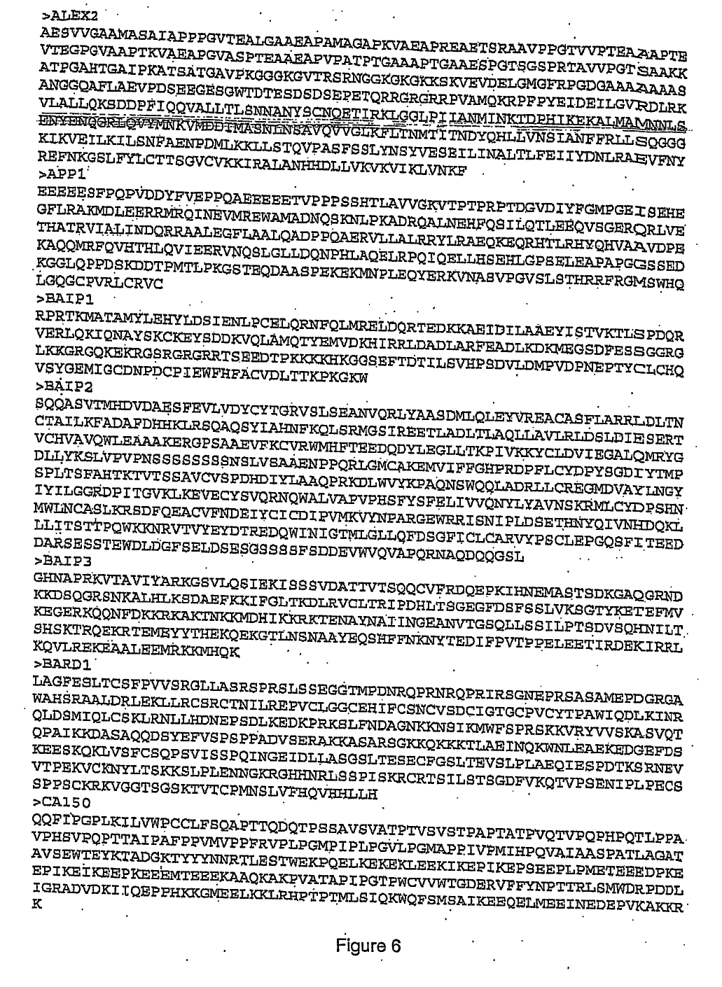

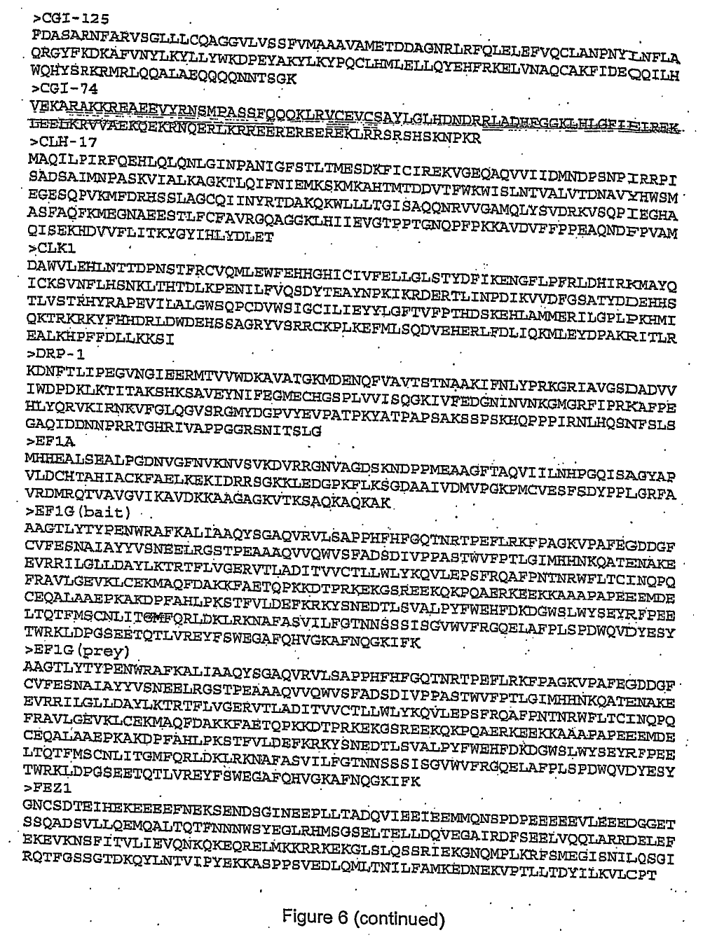

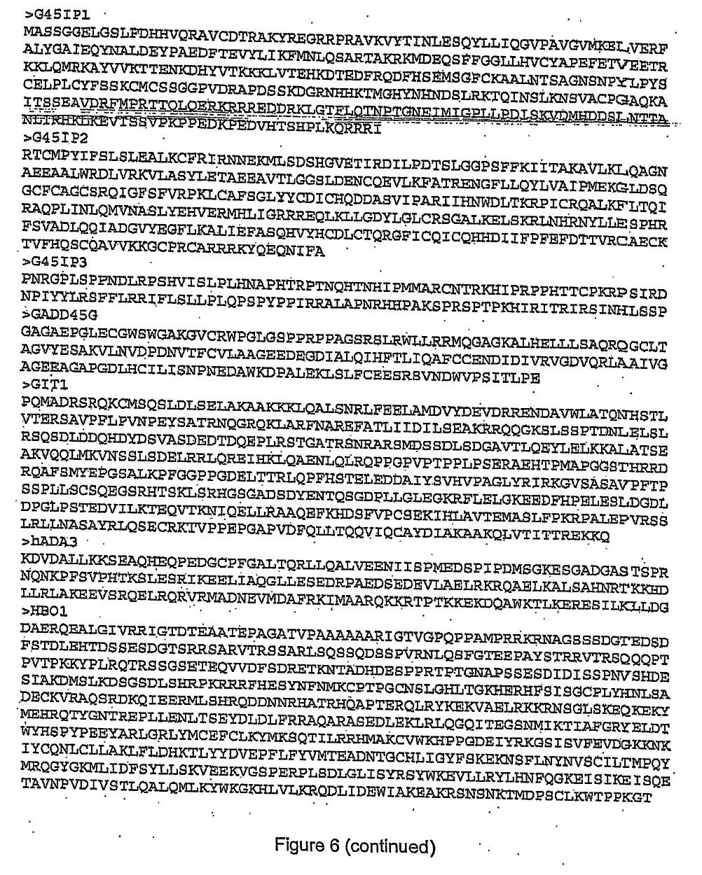

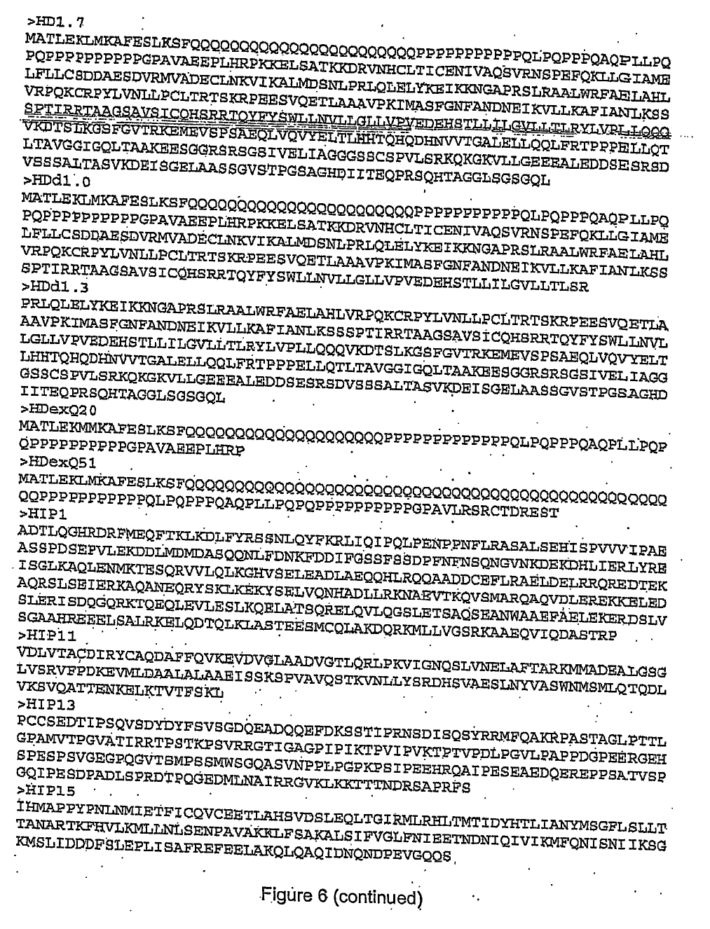

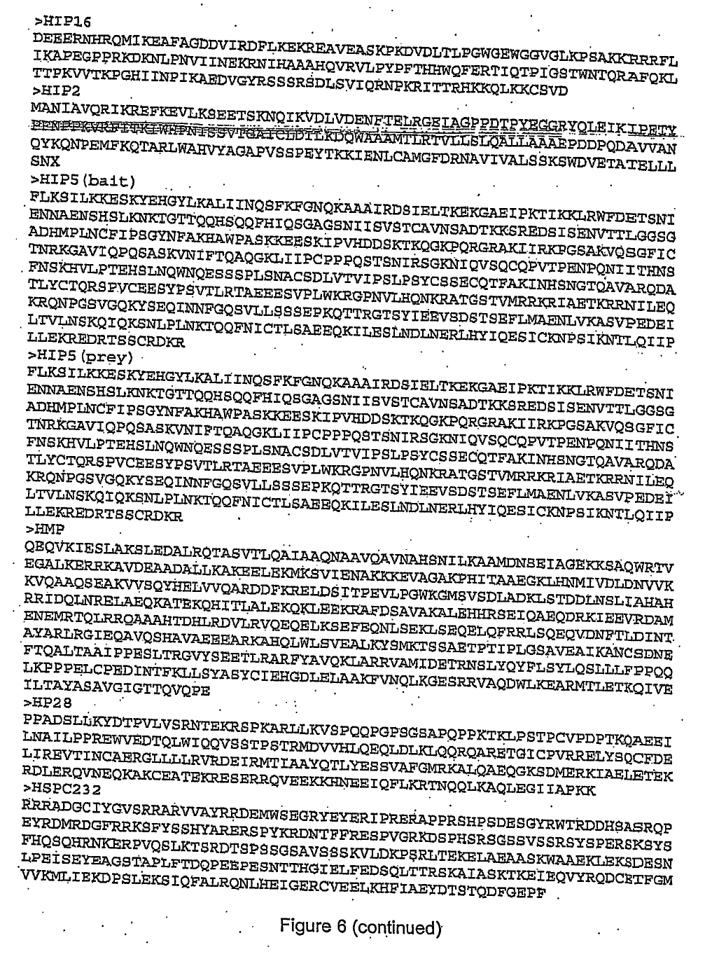

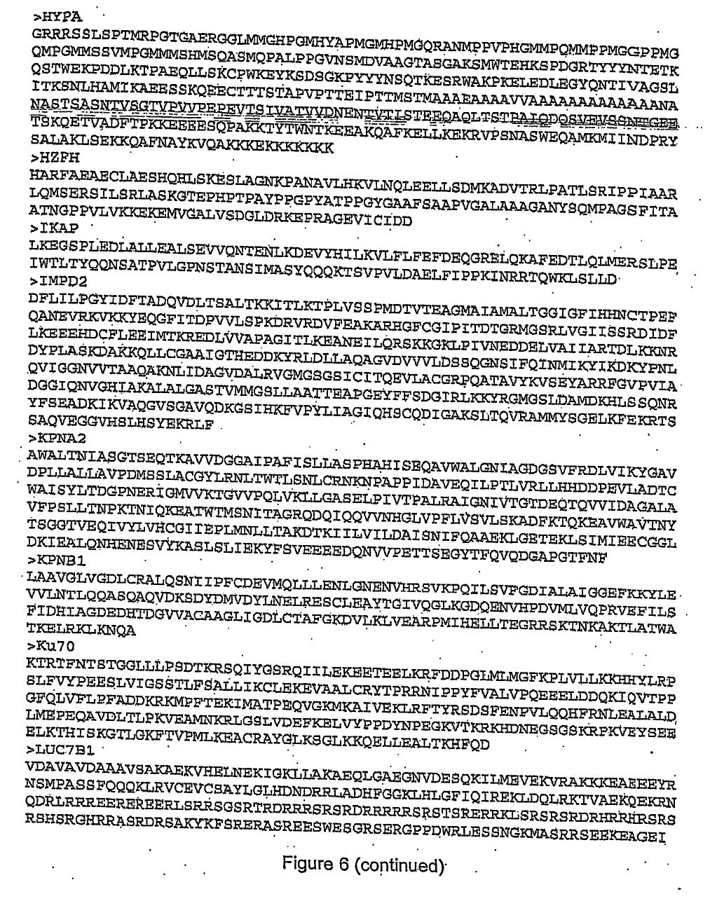

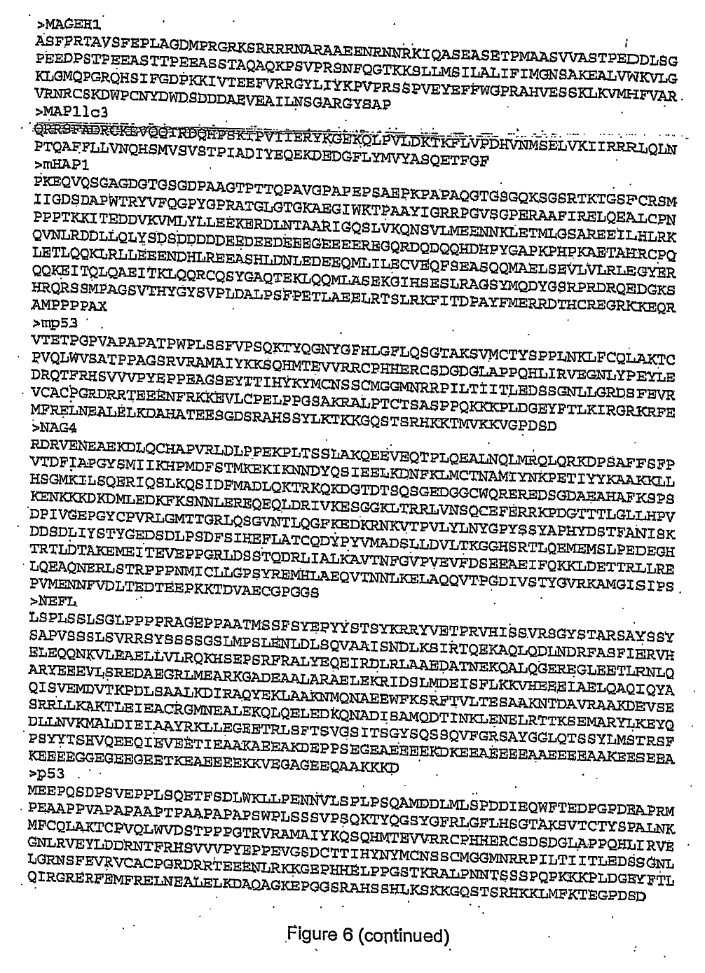

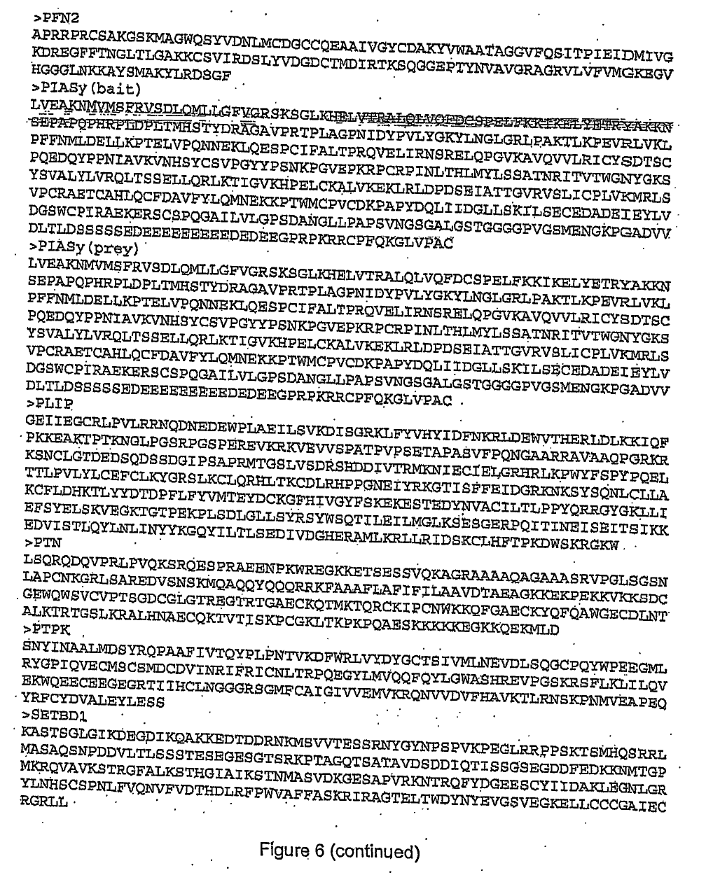

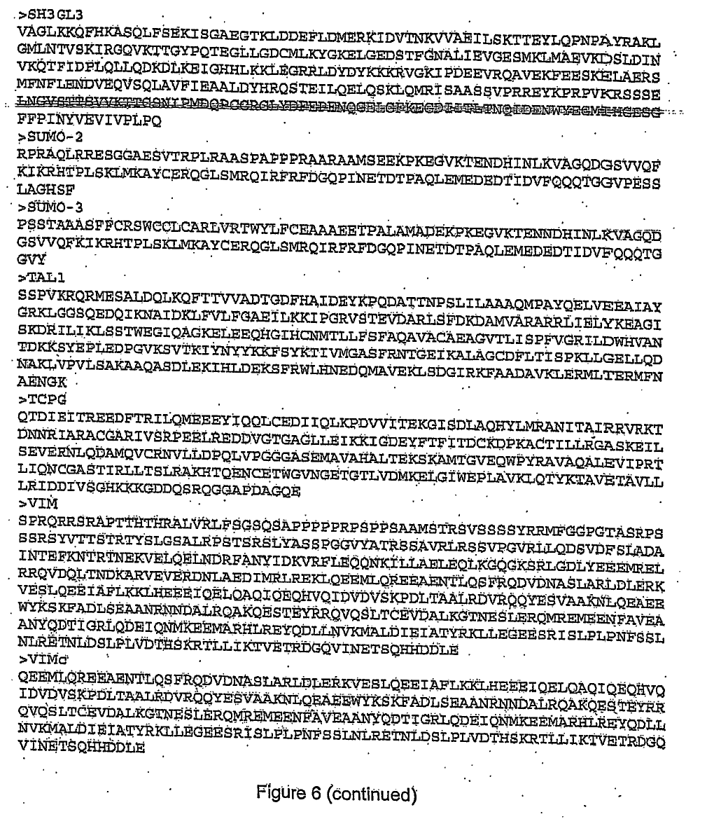

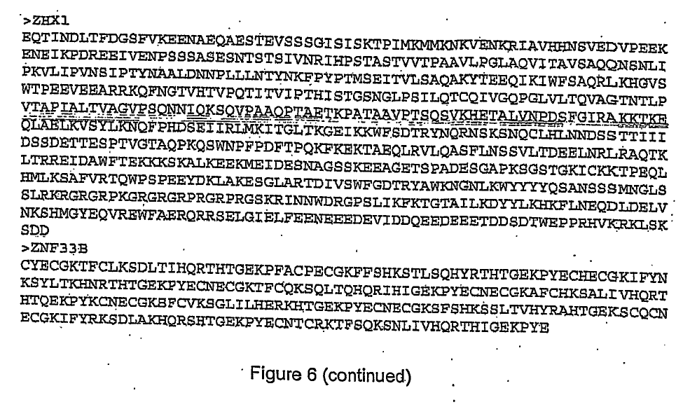

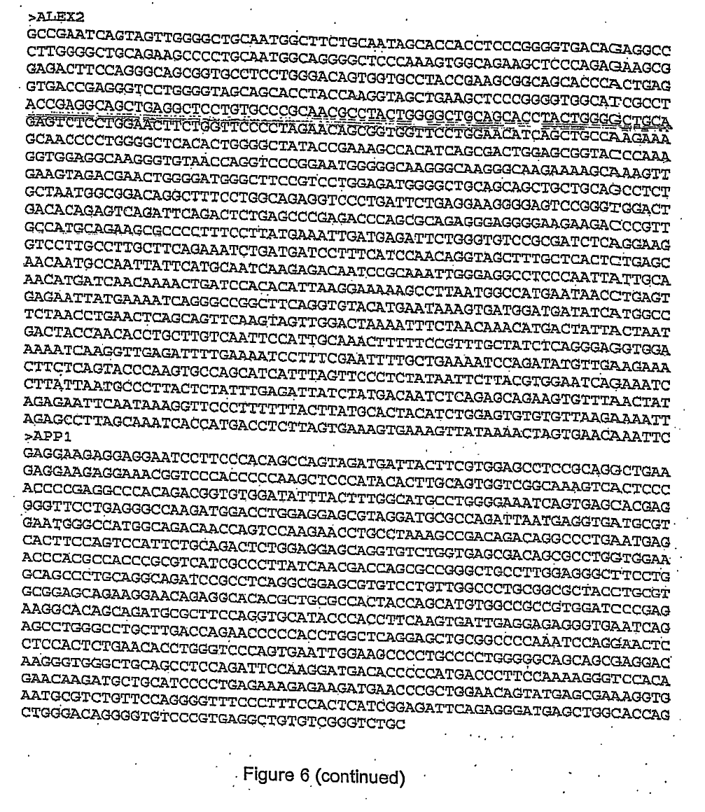

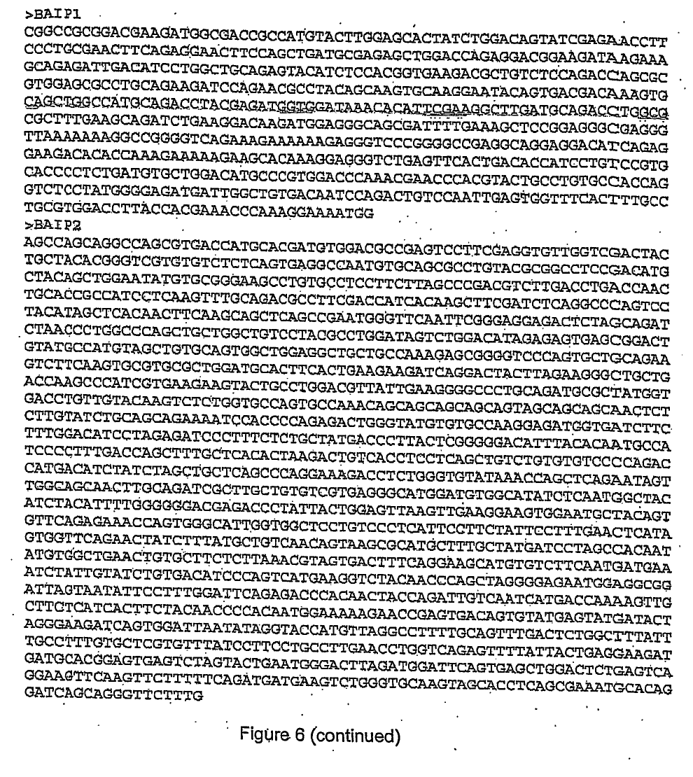

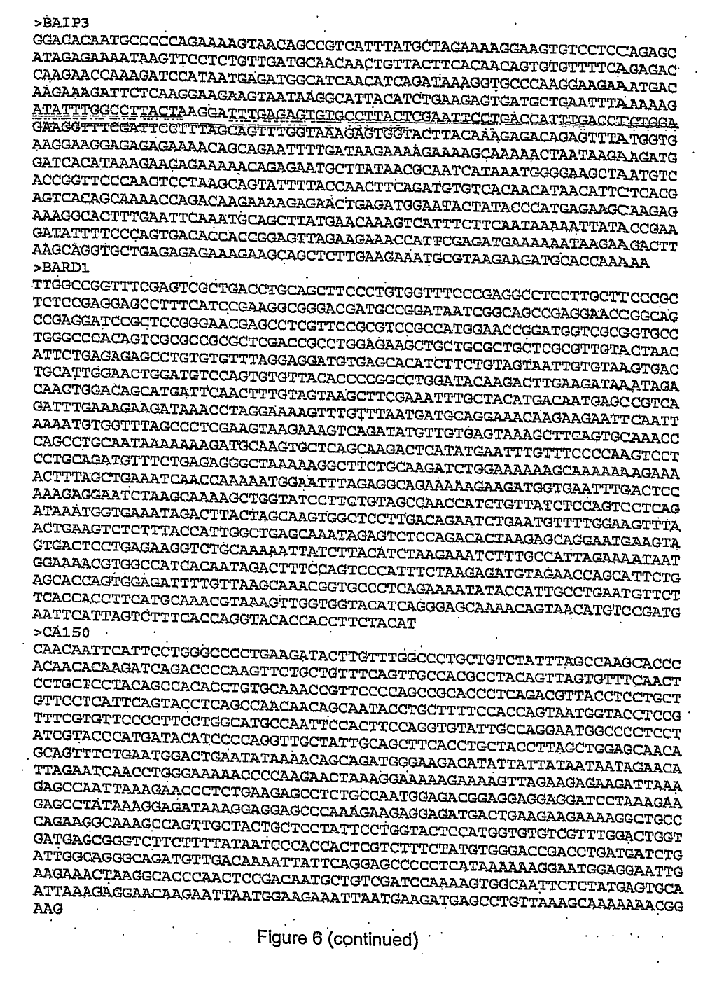

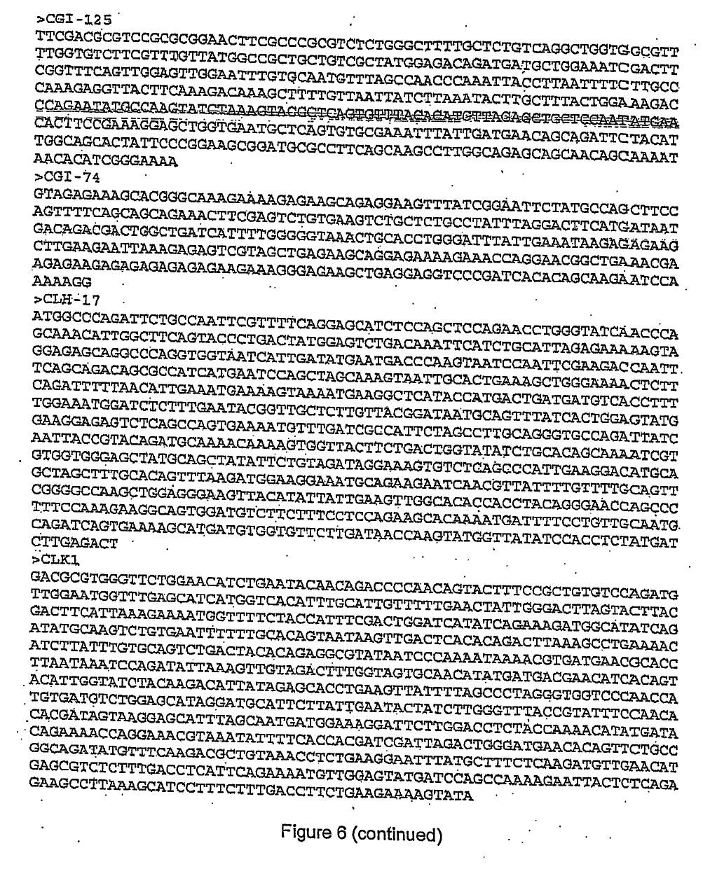

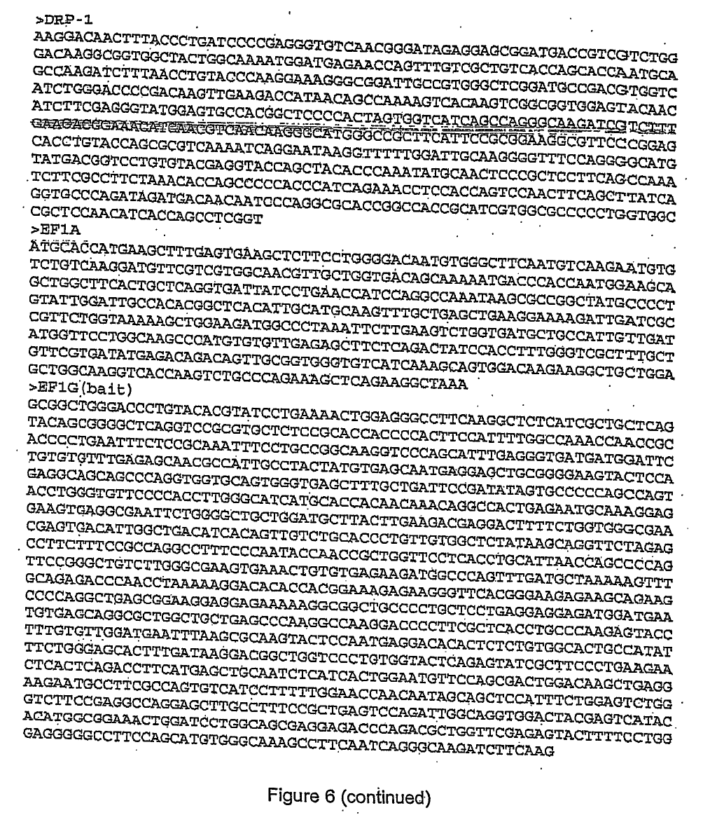

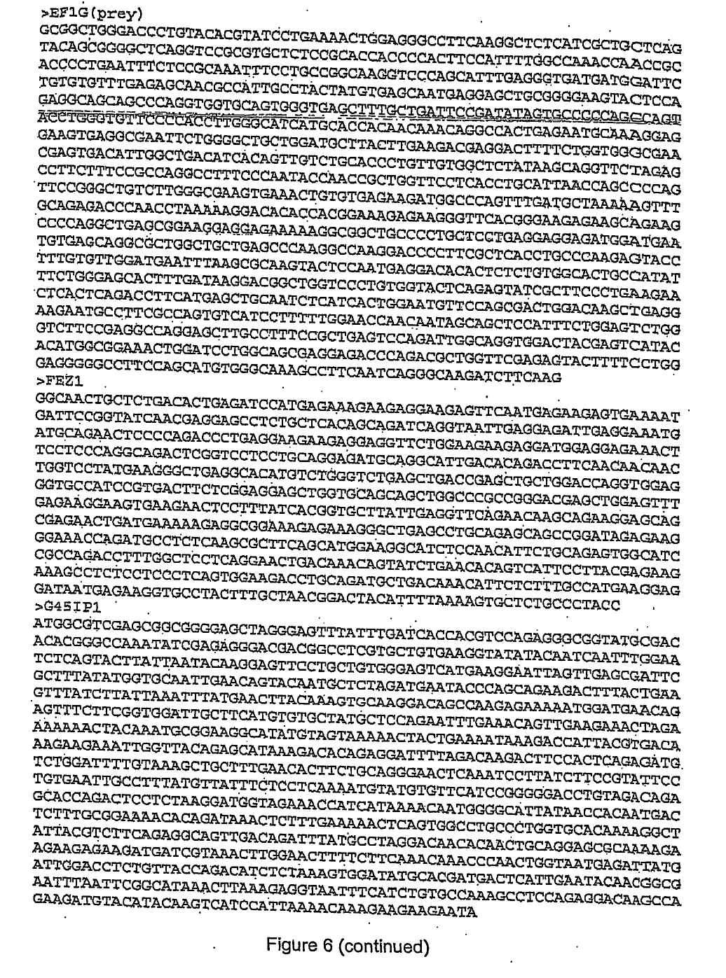

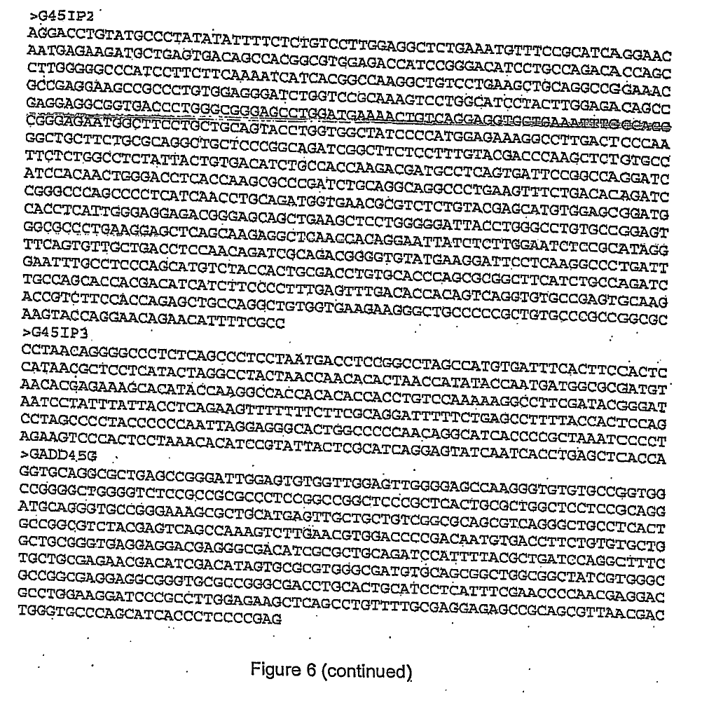

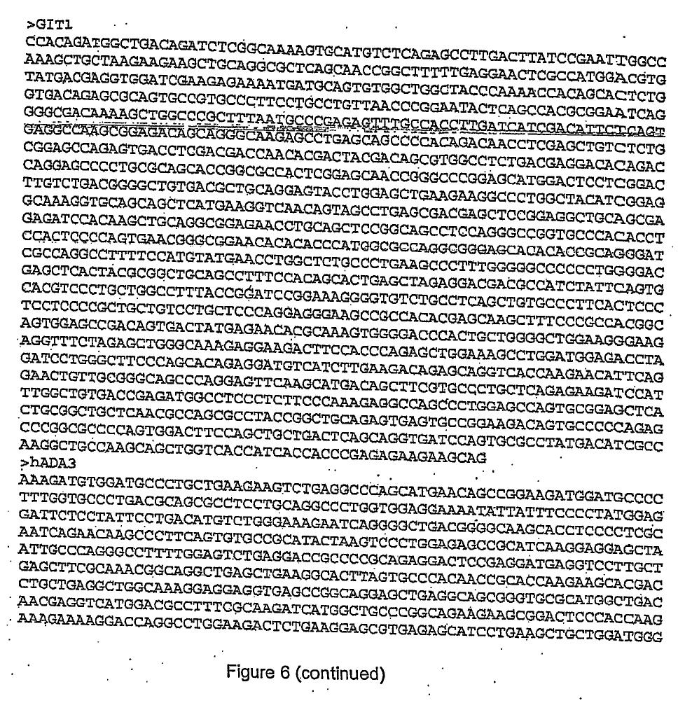

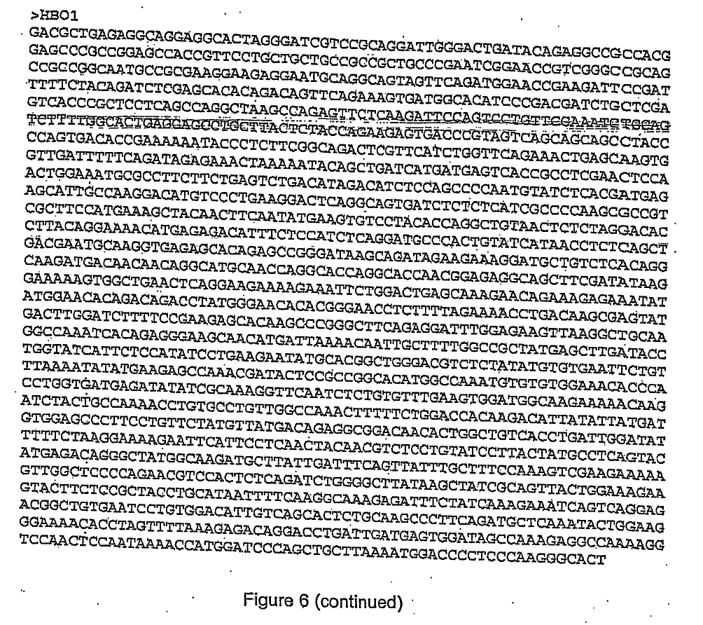

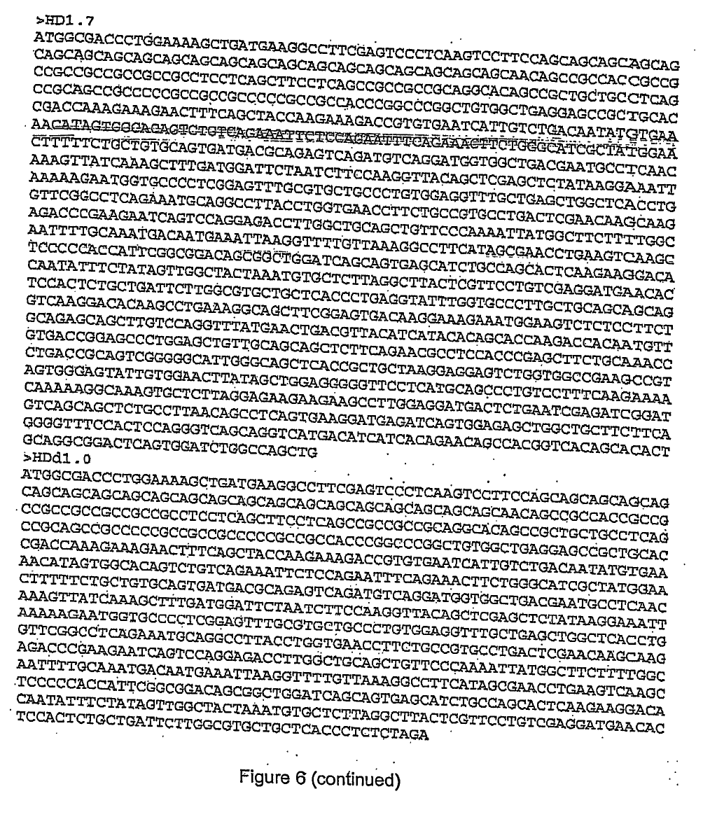

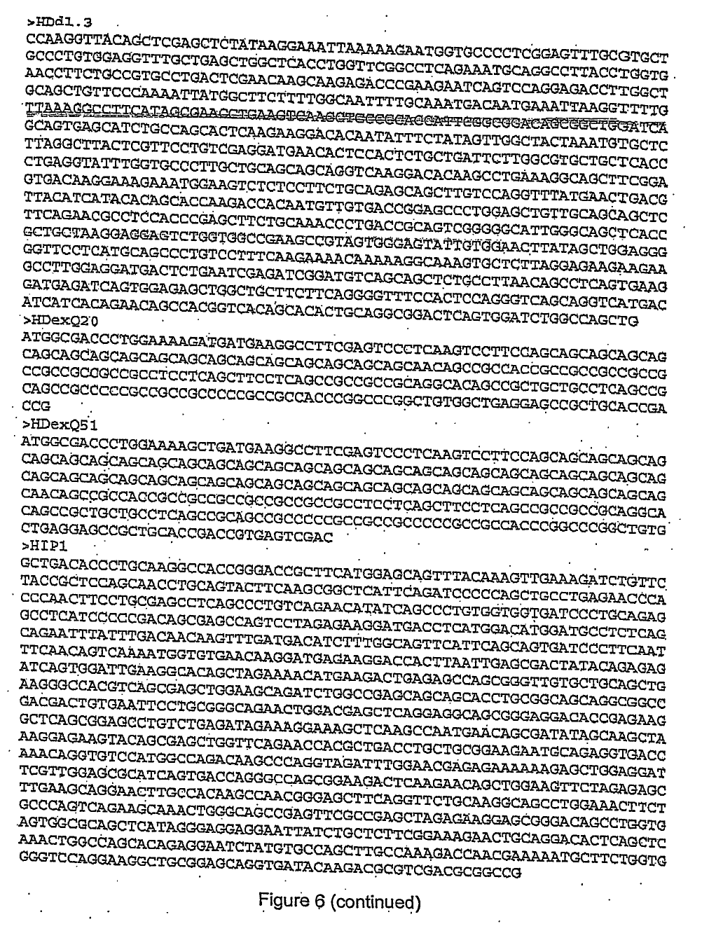

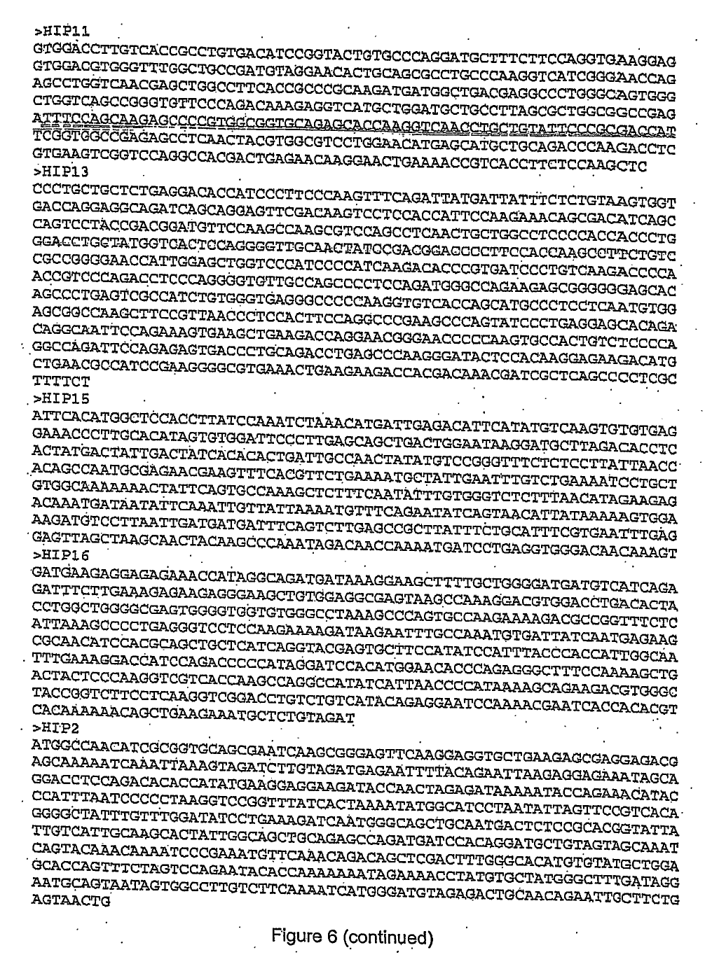

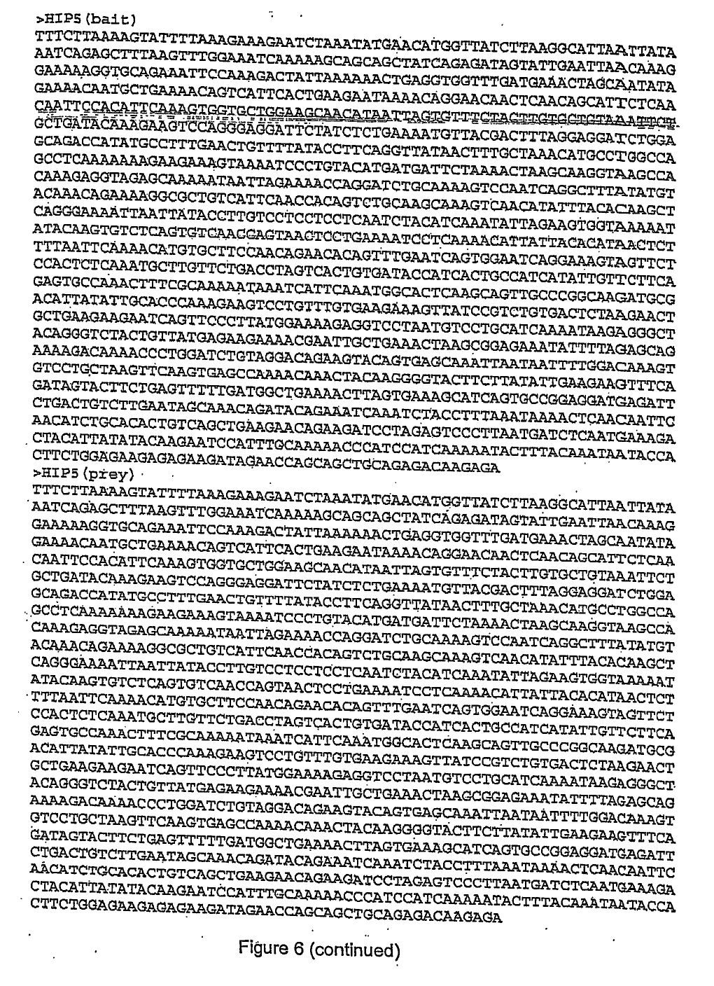

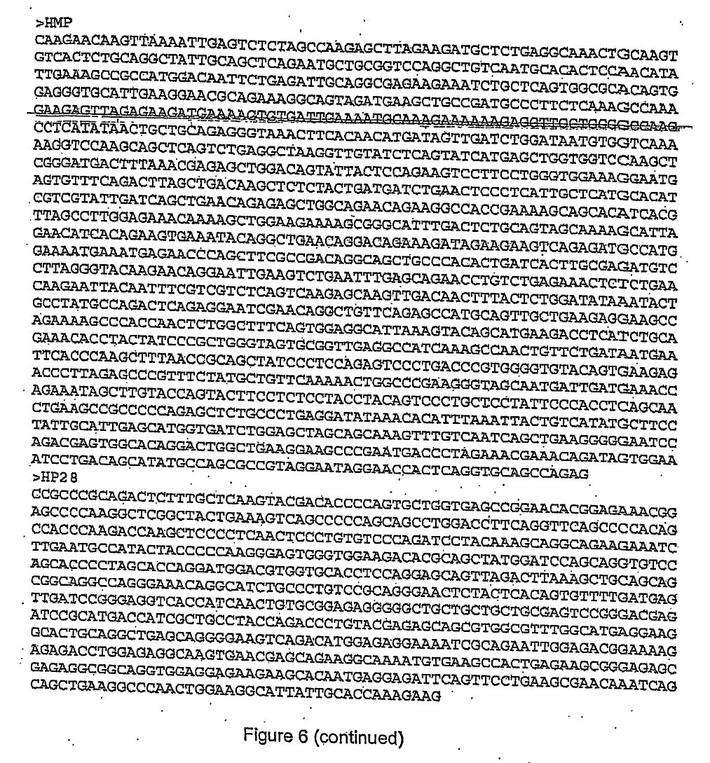

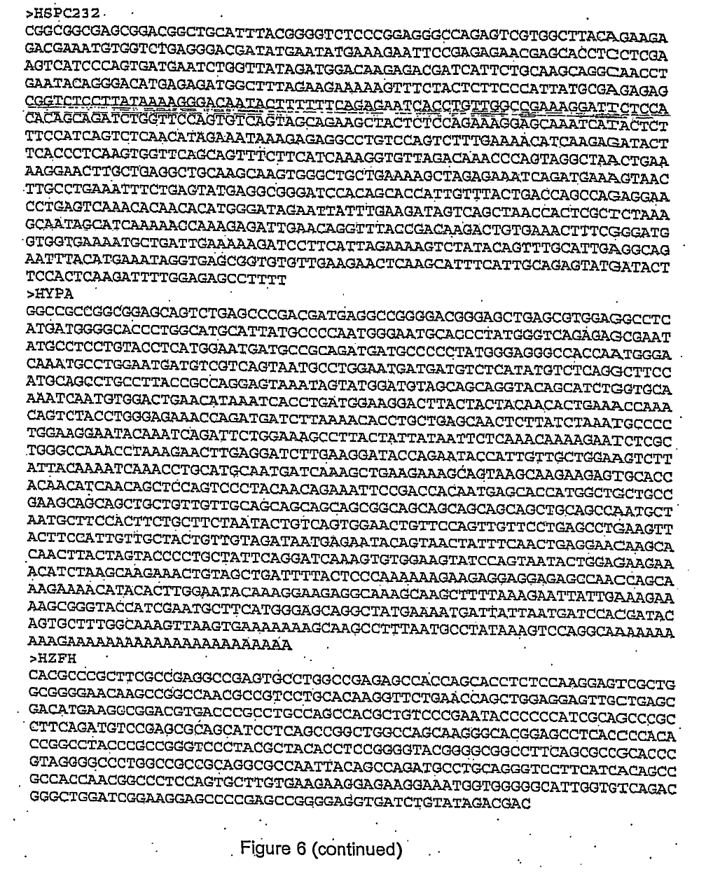

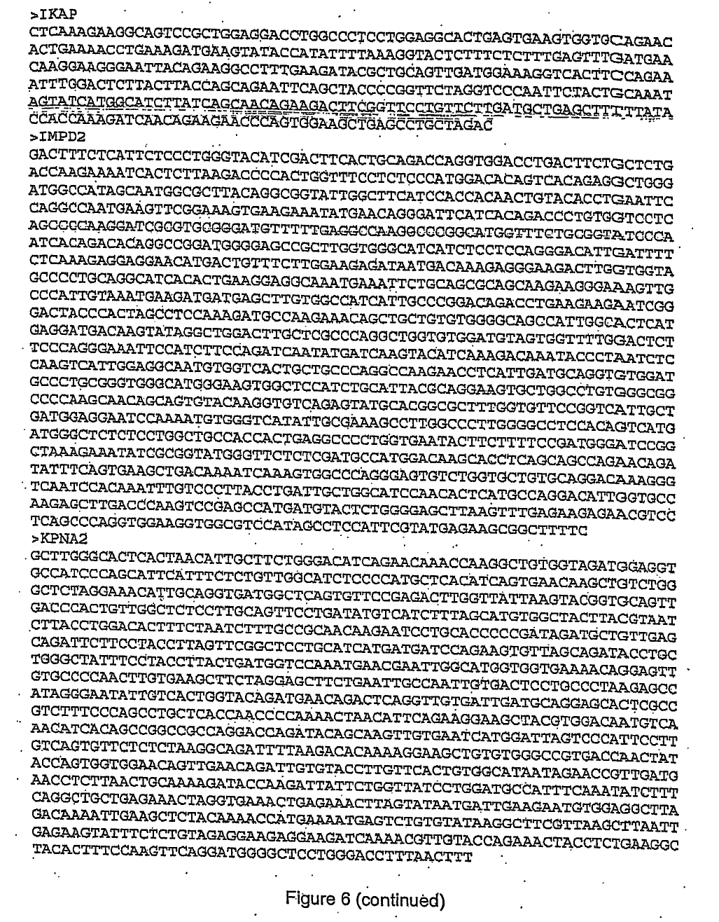

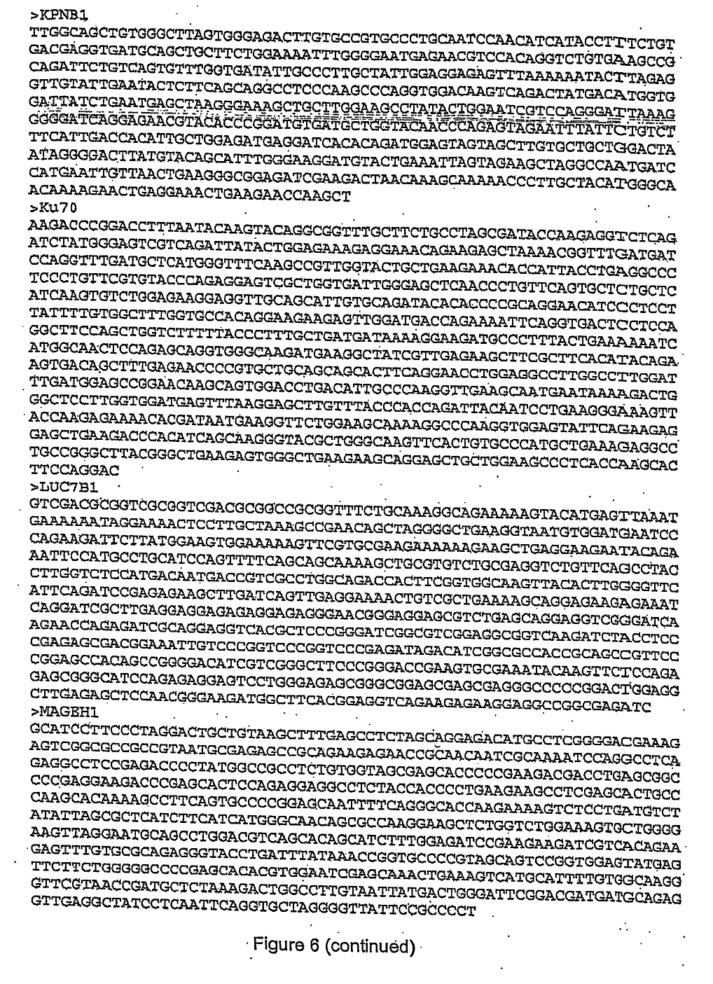

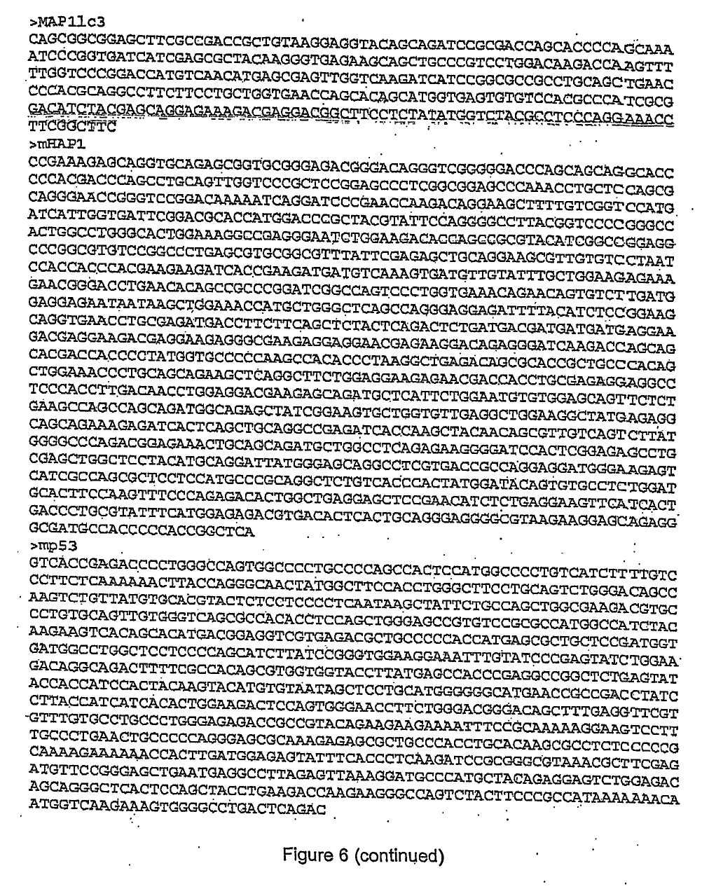

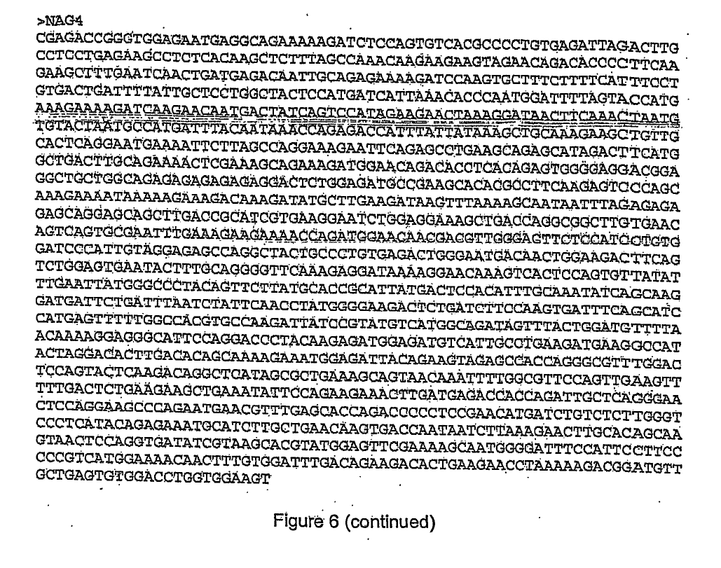

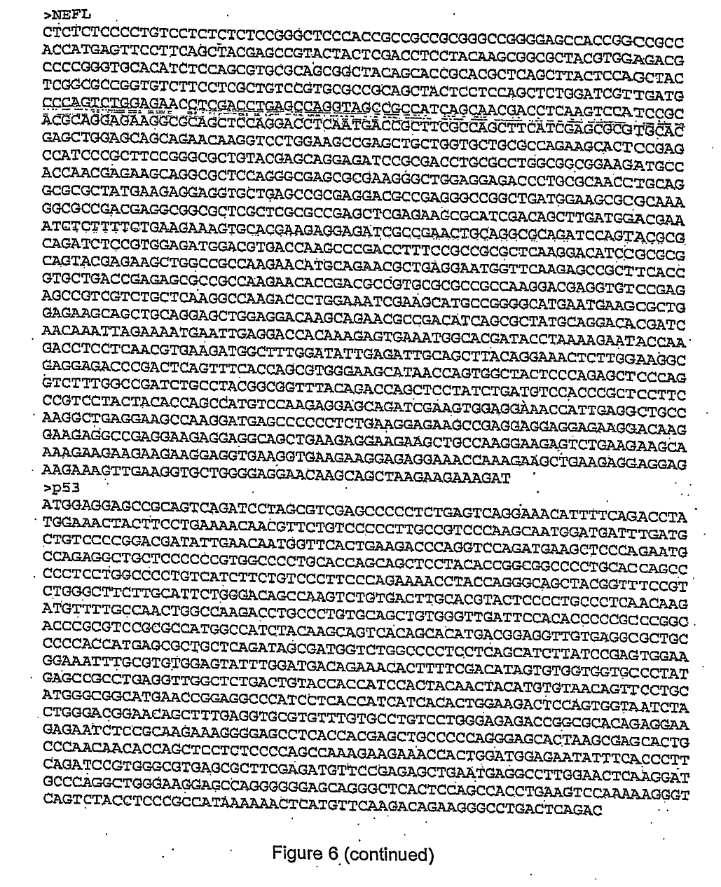

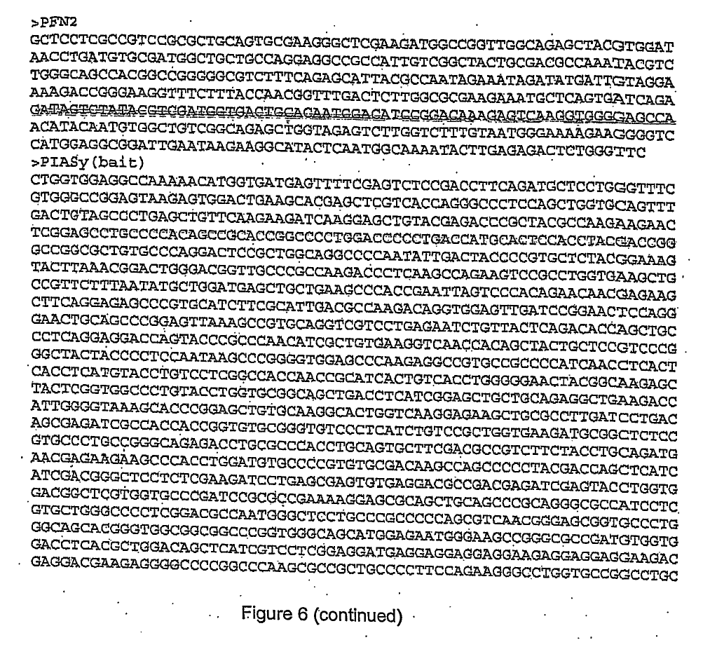

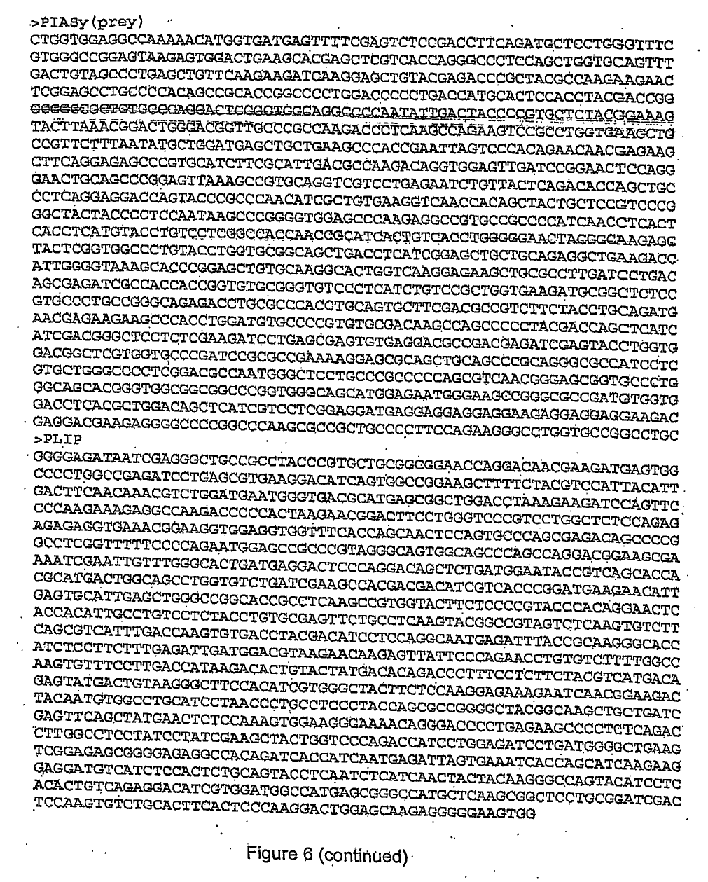

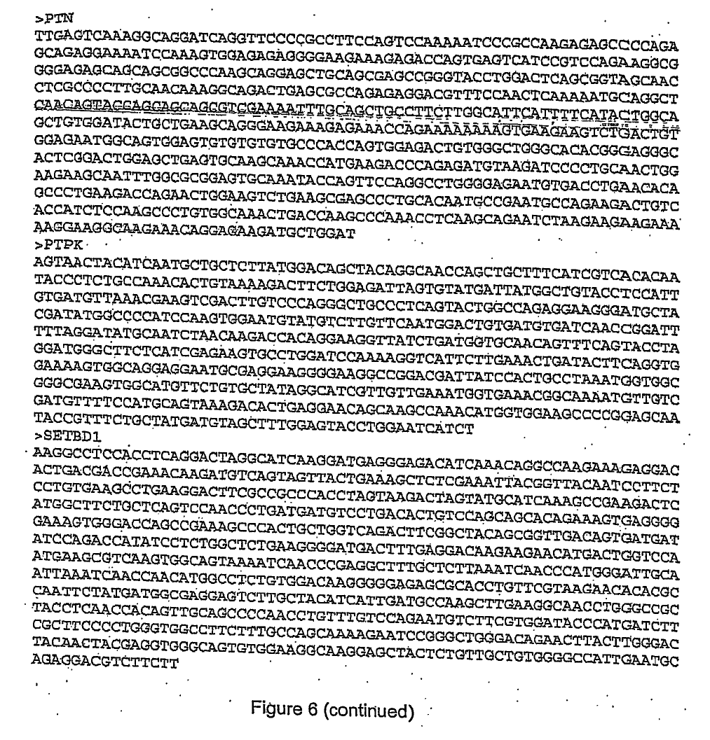

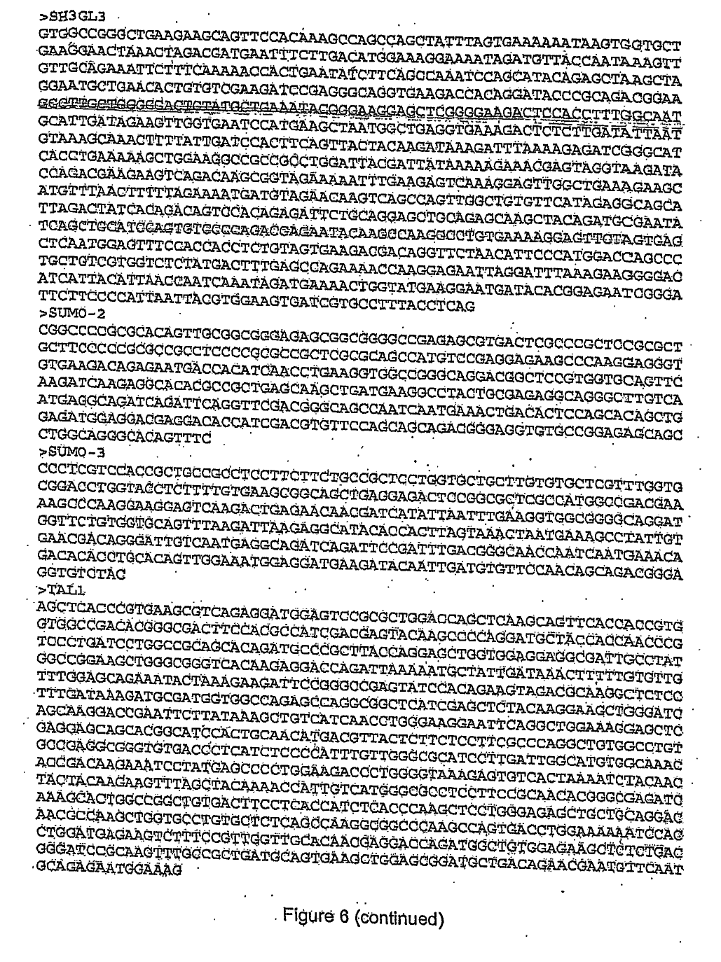

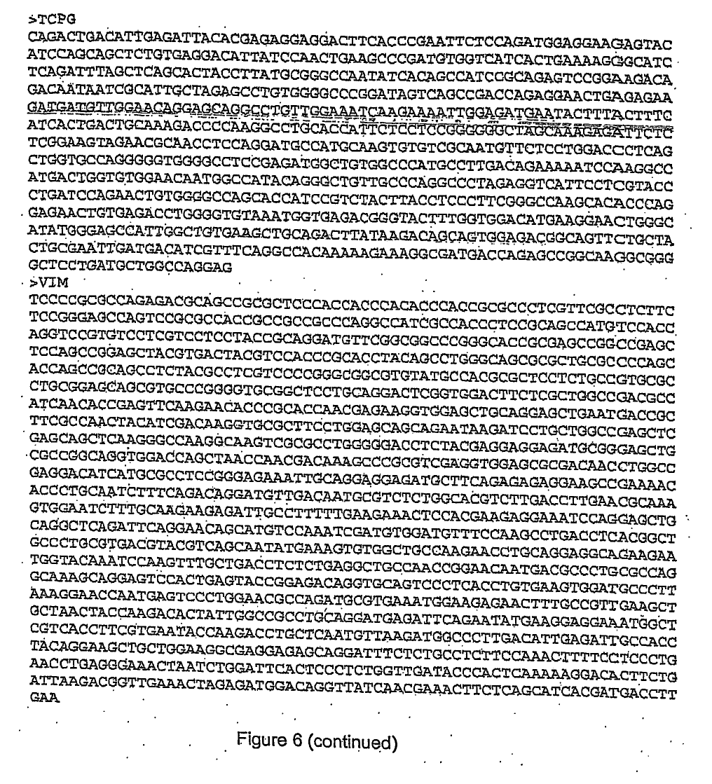

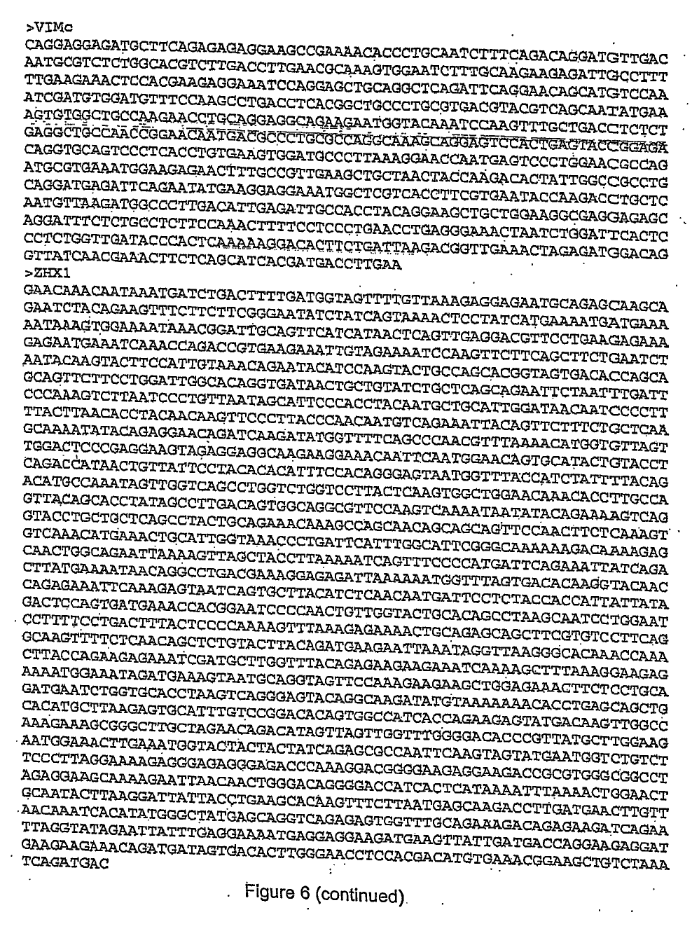

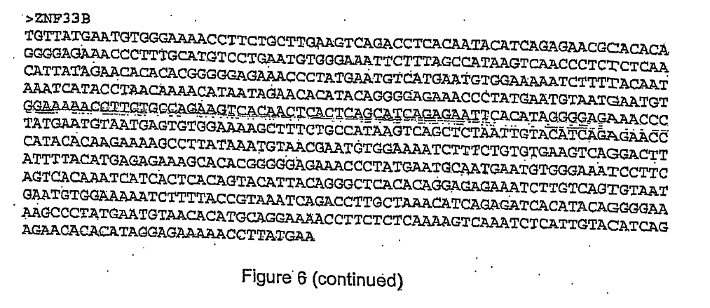

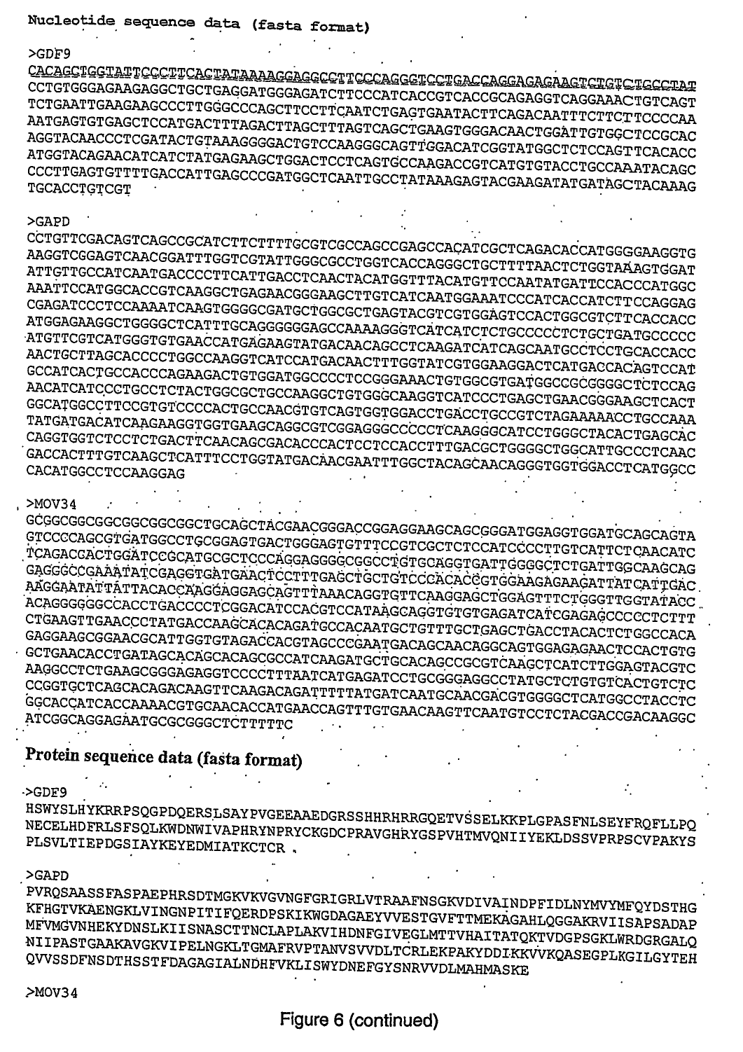

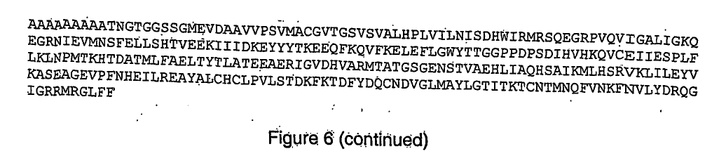

By using the methods disclosed herein, a rate of success or fidelity of at least 70% validatable protein-protein interactions (PPI) (of proteins within the protein interaction network of huntingtin) can be achieved. This level of consistency is well above the level described in the art. In order to increase the rate of success or fidelity, the skilled person can, when carrying out the methods of the present invention, combine the methods of the present invention with additional steps of testing. For example, a step of co-immunoprecipitation and/or an in vitro binding assay may be carried out, in cases when initially the interaction was determined by using the yeast-two-hybrid system (or vice versa). Such additional steps may be carried out at any stage of the methods of the present invention. For example, after but also prior to step (f) of the method of the present invention, PPIs may be verified using in-vitro binding and/or immunoprecipatation assays in order to increase the stringency of the method. By performing these additional steps of testing, the skilled person can increase the rate of success or fidelity to at least 50%, more preferably to at least 60%. For the additional validation, any method may be employed that is available to the skilled artisan for testing the protein interaction. For example, the skilled artisan may simply repeat the step(s) initially carried out, optionally by (slightly) altering the reaction conditions, preferably to more stringent reaction conditions, i.e. conditions that could be expected to further reduce the number of false positive interactions. Alternatively, a different method may be carried out in the validation process. For example, if the method of the invention employed two hybrid systems, the validation might be carried out by precipitation steps as outlined elsewhere in the specification. Whereas the method of the invention provides valid results without the additional validation step(s), the inclusion of such additional validation steps may be advantageous for certain purposes, e.g. drug target identification. In the case that a first validation step does not confirm that the protein in question is a member of the interaction network, further steps in this regard should be carried out. For example, it should be excluded that the validation step(s) do/does not catch weak protein interactions that nevertheless are part of the network. The present invention also relates to a nucleic acid molecule encoding a modulator of huntingtin, wherein said modulator is a protein selected from table 8. FIG. 6 provides the amino acid sequences of the new proteins or (poly)peptides listed in table 8. The term “modulator protein of huntingtin” comprises two types of proteins within the network of proteins interacting with huntingtin. Direct interaction or binding partners of huntingtin are those proteins in the PPI network of huntingtin that directly interact with or bind to huntingtin (see FIG. 2). Examples of these proteins are IKAP, HYPA, CA150, HIP1, HIP11, HIP13, HIP15, CGI-125, PFN2, HP28, DRP-1, SH3GL3, HZFH, HIP5, PIASy, HIP16, GIT1, Ku70 and FEZ1. Table 7 and FIG. 6 provides a reference allowing to identify these proteins. The second class of proteins are indirect interaction or binding partners of huntingtin, i.e. those proteins in the PPI network of huntingtin that do not directly interact with or bind to huntingtin. Such proteins require a mediator, i.e. a direct binding partner of huntingtin to exert their huntingtin modulating function. Examples of these proteins are BARD1 or VIM, which bind to direct interaction partners of huntingtin. However, complexes of huntingtin and a direct interaction or binding partner are likely to interact with additional indirect interaction or binding partners. To summarize the above, modulator proteins of huntingtin can exert their function by direct or indirect contact to huntingtin.

The term “modulator protein”, as used in the present invention, refers to a protein capable of modulating the function or physical state of a second protein and comprises proteins that enhance or reduce (inhibit) the function or activity of huntingtin. Preferably, the modulator protein is a protein having an activity selected from the group consisting of oxidoreductase activity (acting on the CH—OH group of donors, acting on the aldehyde or oxo group of donors, acting on the CH—CH group of donors, acting on the CH—NH(2) group of donors, acting on the CH—NH group of donors, acting on NADH or NADPH, acting on other nitrogenous compounds as donors, acting on a sulfur group of donors, acting on a heme group of donors, acting on diphenols and related substances as donors, acting on a peroxide as acceptor, acting on hydrogen as donor, acting on single donors with incorporation of molecular oxygen, acting on the CH—OH group of donors, acting on superoxide as acceptor, oxidizing metal ions, acting on —CH(2) groups, acting on iron-sulfur proteins as donors, acting on reduced flavodoxin as donor, acting on phosphorus or arsenic in donors, acting on x-H and y-H to form an x-y bond, other oxidoreductases), transferase activity (transferring one-carbon groups, transferring aldehyde or ketone residues, acyltransferases, glycosyltransferases, transferring alkyl or aryl groups, other than methyl groups, transferring nitrogenous groups, transferring phosphorous-containing groups, transferring sulfur-containing groups, transferring selenium-containing groups), hydrolase activity (glycosylase activity, acting on ether bonds, acting on peptide bonds, acting on carbon-nitrogen bonds (other than peptide bonds), acting on acid anhydrides, acting on carbon-carbon bonds, acting on halide bonds, acting on phosphorus-nitrogen bonds, acting on sulfur-nitrogen bonds, acting on carbon-phosphorus bonds, acting on sulfur-nitrogen bonds, acting on carbon-phosphorus bonds, acting on sulfur-sulfur bonds, acting on carbon-sulfur bonds, lyases (carbon-carbon lyases, carbon-oxygen lyases, carbon-nitrogen lyases, carbon-sulfur lyases, carbon-halide lyases, phosphorus-oxygen lyases, other lyases), isomerases (racemases and epimerases, cis-trans-isomerases, intramolecular oxidoreductases, intramolecular transferases, intramolecular lyases, other isomerases), ligases activity (forming carbon-oxygen bonds, forming carbon-sulfur bonds, forming carbon-nitrogen bonds, forming carbon-carbon bonds, forming phosphoric ester bonds), transcription factor activity, filament protein, membrane protein and structural protein.

In a preferred embodiment, the present invention's nucleic acid molecule is DNA, or RNA, and preferably cDNA, or genomic DNA or synthetic DNA or mRNA

In another preferred embodiment of the invention, the nucleic acid molecule is double stranded or single stranded.

In another preferred embodiment of the invention, the nucleic acid molecule is of vertebrate, nematode, insect, bakterium or yeast. Preferably, the nematode is Caenorhabditis elegans. In another more preferred embodiment of the present invention, the insect is drosophila, preferably drosiphila melanogaster. In another more preferred embodiment of the present invention, the vertebrate is human, mouse rat, Xenopus laevis, zebrafish.

In yet another preferred embodiment of the present invention, the nucleic acid molecule is fused to a heterologous nucleic acid molecule. In a further preferred embodiment of the present invention, the heterologous (poly)peptide encoded by said heterlogous nucleic acid molecule is an immunoglobulin Fc domain.

In another preferred embodiment of the present invention the nucleic acid molecule is labeled. Labeled nucleic acid molecules may be useful for purification or detection. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine(ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may also be a two stage system, where the DNA is conjugated to biotin, haptens, etc. having a high affinity binding partner, e.g. avidin, specific antibodies, etc., where the binding partner is conjugated to a detectable label. In the case of amplification the label may be conjugated to one or both of the primers. The pool of nucleotides used in the amplification may also be labeled, so as to incorporate the label into the amplification product. Alternatively, the double strand formed after hybridization can be detected by anti-double strand DNA specific antibodies or aptamers etc.

In a more preferred embodiment said heterologous nucleic acid molecule encodes a heterologous polypeptide. Preferably said heterologous (poly)peptide, fused to the (poly)peptide encoded by the nucleic acid molecule of the present invention, is a DNA binding protein selected from the group consisting of GAL4 (DBP) and LexA (DBP). Also preferred in accordance with the present invention are activation domains selected from the group consisting of GAL4(AD) and VP16(AD). Also, preferred are (poly)peptides selected from the group consisting of GST, His Tag, Flag Tag, Tap Tag, HA Tag and Protein A Tag.

Thus, the sequence encoding the (poly)peptide may be fused to a marker sequence, such as a sequence encoding a peptide which facilitates purification of the fused (poly)peptide. In certain preferred embodiments of this aspect of the invention, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. The “HA” tag is another peptide useful for purification which corresponds to an epitope derived from the influenza hemagglutinin protein, which has been described by Wilson et al., Cell 37: 767 (1984).

The (poly)peptide may be expressed in a modified form, such as a fusion protein, and may include not only secretion signals, but also additional heterologous functional regions. For instance, a region of additional amino acids, particularly charged amino acids, may be added to the N-terminus of the (poly)peptide to improve stability and persistence in the host cell, during purification, or during subsequent handling and storage. Also, peptide moieties may be added to the (poly)peptide to facilitate purification. Such regions may be removed prior to final preparation of the (poly)peptide. The addition of peptide moieties to (poly)peptides to engender secretion or excretion, to improve stability and to facilitate purification, among others, are familiar and routine techniques in the art. A preferred fusion protein comprises a heterologous region from immunoglobulin that is useful to stabilize and purify proteins.

The present invention also relates to a method of producing a vector comprising the nucleic acid molecule the present invention. Furthermore, the present invention relates to a vector produced said method.

The present invention also relates to a vector comprising the nucleic acid molecule of the present invention. Preferably said vector is a transfer or expression vector selected from the group consisting of pACT2; pAS2-1; pBTM116; pBTM117; pcDNA3.1; pcDNAI; pECFP; pECFP-C1; pECFP-N1; pECFP-N2; pECFP-N3; pEYFP-C1; pFLAG-CMV-5 a, b, c; pGAD10; pGAD424; pGAD425; pGAD427; pGAD428; pGBT9; pGEX-3×1; pGEX-5×1; pGEX-6P1; pGFP; pQE30; pQE30N; pQE30-NST; pQE31; pQE31 N; pQE32; pQE32N; pQE60; pSE111; pSG5; pTET-CMV-AS; pTET-CMV-F.°-AS; pTET-CMV-F.°-S; pTET-CMV-MCS; pTET-CMV-S; pTK-Hyg; pTL1; pTL10; pTL-HA0; pTL-HA1; pTL-HA2; pTL-HA3; pBTM118c; pGEX-6P3; pACGHLT-C; pACGHLT-A; pACGHLT-B; pUP; pcDNA3.1-V5His; pMalc2x. Said expression vectors may particularly be plasmids, cosmids, viruses or bacteriophages used conventionally in genetic engineering plasmids, cosmids, viruses and bacteriophages used conventionally in genetic engineering that comprise the aforementioned nucleic acid. Preferably, said vector is a gene transfer or targeting vector. Expression vectors derived from viruses such as retroviruses, vaccinia virus, adeno-associated virus, herpes viruses, or bovine papilloma virus, may be used for delivery of the nucleic acid into targeted cell population. Methods which are well known to those skilled in the art can be used to construct recombinant viral vectors; see, for example, the techniques described in Sambrook et al., Molecular Cloning A Laboratory Manual, Cold Spring Harbor Laboratory (1989) N.Y. and Ausubel et al., Current Protocols in Molecular Biology, Green Publishing Associates and Wiley Interscience, N.Y. (1989).

In yet a further preferred embodiment of the invention the vector contains an additional expression cassette for a reporter protein, selected from the group consisting of β-galactosidase, luciferase, green fluorescent protein and variants thereof.

Preferably, said vector comprises regulatory elements for expression of said nucleic acid molecule. Consequently, the nucleic acid of the invention may be operatively linked to expression control sequences allowing expression in eukaryotic cells. Expression of said nucleic acid molecule comprises transcription of the sequence nucleic acid molecule into a translatable mRNA. Regulatory elements ensuring expression in eukaryotic cells, preferably mammalian cells, are well known to those skilled in the art. They usually comprise regulatory sequences ensuring initiation of transcription and, optionally, a poly-A signal ensuring termination of transcription and stabilization of the transcript, and/or an intron further enhancing expression of said nucleic acid. Additional regulatory elements may include transcriptional as well as translational enhancers, and/or naturally-associated or heterologous promoter regions. Possible regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the nucleic acid molecule. Furthermore, depending on the expression system used leader sequences capable of directing the (poly)peptide to a cellular compartment or secreting it into the medium may be added to the coding sequence of the aforementioned nucleic acid and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an C- or N-terminal identification peptide imparting desired characteristics, e.g., stabilization or simplified purification of expressed recombinant product. In this context, suitable expression vectors are known in the art such as Okayama-Berg cDNA expression vector pcDVI (Pharmacia), pCDM8, pRc/CMV, pcDNA1, pcDNA3, the Echo™ Cloning System (Invitrogen), pSPORT1 (GIBCO BRL) or pRevTet-On/pRevTet-Off or pCI (Promega).

The present invention also relates to a method of producing a host cell comprising genetically engineering cells with the nucleic acid molecule or the vector of the present invention. The present invention also relates to a host cell produced said method. Furthermore, the present invention relates to a host cell comprising the vector of the present invention. Preferably, said host cell contains an endogenous nucleic acid molecule which is operably associated with a heterologous regulatory control sequence, including the regulatory elements contained in the vector of the present invention.

The present invention also relates to a method of producing a (poly)peptide, comprising culturing the host cell of the present invention under conditions such that the (poly)peptide encoded by said polynucleotide is expressed and recovering said (poly)peptide.

The present invention also relates to a (poly)peptide comprising an amino acid sequence encoded by a nucleic acid molecule of the present invention, or which is chemically synthesized, or is obtainable from the host cell of the present invention, or which is obtainable by a method of the present invention or which is obtainable from an in vitro translation system by expressing the nucleic acid molecule of the present invention or the vector of the present invention.

In another preferred embodiment of the invention, the (poly)peptide or protein is of vertebrate, nematode, insect, bakterium or yeast. Preferably, the nematode is Caenorhabditis elegans. In another more preferred embodiment of the present invention, the insect is Drosophila, preferably Drosophila melanogaster. In another more preferred embodiment of the present invention, the vertebrate is human, mouse rat, Xenopus laevis, zebrafish.

In another preferred embodiment, the (poly)peptide of the present invention is fused to a heterologous (poly)peptide. Such a fusion protein may include not only secretion signals, but also additional heterologous functional regions. For instance, a region of additional amino acids, particularly charged amino acids, may be added to the N-terminus of the (poly)peptide to improve stability and persistence in the host cell, during purification, or during subsequent handling and storage. Also, peptide moieties may be added to the (poly)peptide to facilitate purification. Such regions may be removed prior to final preparation of the (poly)peptide. The addition of peptide moieties to (poly)peptides to engender secretion or excretion, to improve stability and to facilitate purification, among others, are familiar and routine techniques in the art. A preferred fusion protein comprises a heterologous region from immunoglobulin that is useful to stabilize and purify proteins.

In a preferred embodiment of the present invention, the (poly)peptide of the present invention is fused to a heterologous (poly)peptide which is an immunoglobulin Fc domain or Protein A domain. In another preferred embodiment of the present invention, the (poly)peptide the (poly)peptide is labelled. Preferably, the label is selected from the group consisting of fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine(ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may also be a two stage system, where the protein or (poly)peptide is conjugated to biotin, haptens, etc. having a high affinity binding partner, e.g. avidin, specific antibodies, etc., where the binding partner is conjugated to a detectable label. In another preferred embodiment of the present invention the label is a toxin, radioisotope, or fluorescent label.

In another preferred embodiment of the present invention, the (poly)peptide contains or lacks an N-terminal methionine. it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins this prokaryotic removal process is inefficient, depending on the nature of the amino acid to which the N-terminal methionine is covalently linked.

The present invention also relates to a protein complex comprising at least two proteins, wherein said at least two proteins are selected from the group of interaction partners listed in table 9. The term “protein complex” refers to a compound stably comprising at least two proteins. Preferably, said stability allows to purify said protein complex. In a preferred embodiment of the present invention, the protein complex comprises GIT1 and huntingtin.

The present invention also relates to the protein network of huntingtin, preferably the physical protein entities forming this network, which is described herein. In one embodiment, said protein network is formed by the interaction partners shown in table 6. Preferable, the protein network of the present invention is a validated protein network as described herein.

The present invention also relates to an antibody specifically recognizing the (poly)peptide of the present invention or specifically reacting with the protein complex of the present invention. This antibody is characterized in not recognizing the individual components of the protein complex but rather the complex itself. As such, said antibody recognizes a combined epitope, composed of amino acids of two different proteins within the protein complex. Dissociation of the complex will be detrimental to antibody recognition. Therefore, antibody binding depends on the integrity of the protein complex. In a preferred embodiment of the present invention, the antibody is specific for a protein complex comprising GIT1 and huntingtin.

In a preferred embodiment, the antibody of the present invention is polyclonal, monoclonal, chimeric, single chain, single chain Fv, human antibody, humanized antibody, or Fab fragment

In a more preferred embodiment of the present invention the antibody is labeled. Preferably, the label is selected from the group consisting of fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine(ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may also be a two stage system, where the antibody is conjugated to biotin, haptens, etc. having a high affinity binding partner, e.g. avidin, specific antibodies, etc., where the binding partner is conjugated to a detectable label. In another preferred embodiment of the present invention the label is a toxin, radioisotope, or fluorescent label.

In a preferred embodiment of the present invention, the antibody is immobilized to a solid support. Preferably, the solid support may be the surface of a cell, a microtiter plate, beads or the surface of a sensor capable of detecting binding of the antibody or to the antibody.

The present invention also relates to a method of identifying whether a protein promotes huntingtin aggregation, comprising (a) transfecting a first cell with a nucleic acid molecule encoding a variant of the huntingtin protein or a fragment thereof capable of forming huntingtin aggregates; (b) co-transfecting a second cell with (i) a nucleic acid molecule encoding a variant of the huntingtin protein or a fragment thereof capable of forming huntingtin aggregates; and (ii) a nucleic acid molecule encoding a candidate modulator protein identified by the methods of the present invention or a nucleic acid molecule encoding a modulator protein selected from table 6 or table 7 (c) expressing the proteins encoded by the transfected nucleic acid molecule of (a) and (b); (d) isolating insoluble aggregates of huntingtin from the transfected cell of (a) and (b); and (e) determining the amount of insoluble huntingtin aggregates from the transfected cell of (a) and (b), wherein an increased amount of huntingtin aggregates isolated from the transfected cells of (b) in comparison with the amount of huntingtin aggregates isolated from the transfected cells of (a) is indicative of a protein's activity as an enhancer of huntingtin aggregation. Preferably, the huntingtin protein or protein fragment of step (a) is HD169Q68 or HD510Q68.

The present invention also relates to a method of identifying whether a protein inhibits huntingtin aggregation, comprising (a) transfecting a first cell with a nucleic acid molecule encoding a variant of the huntingtin protein or a fragment thereof capable of forming huntingtin aggregates; (b) co-transfecting a second cell with (i) a nucleic acid molecule encoding a variant of the huntingtin protein or a fragment thereof capable of forming huntingtin aggregates; and (ii) a nucleic acid molecule encoding a candidate modulator protein identified by the methods of the present invention or a nucleic acid molecule encoding a modulator protein selected from table 6 or table 7 (c) expressing the proteins encoded by the transfected nucleic acid molecule of (a) and (b); (d) isolating insoluble aggregates of huntingtin from the transfected cell of (a) and (b); and (e) determining the amount of insoluble huntingtin aggregates from the transfected cell of (a) and (b), wherein a reduced amount of huntingtin aggregates isolated from the transfected cells of (b) in comparison with the amount of huntingtin aggregates isolated from the transfected cells of (a) is indicative of a protein's activity as an inhibitor of huntingtin aggregation. Preferably, the huntingtin protein or protein fragment of step (a) is HD169Q68 or HD510Q68 or HdexQ51.

The term “promotes” means increasing the amount of huntingtin aggregation.

Preferably said huntingtin protein or the fragments thereof is selected from the proteins listed in table 6 and/or 7. Preferably said insoluble aggregates are isolated by using a filter retardation method comprising lysing cells and boiling in 2% SDS for 5 min in the presence of 100 mM DDT followed by a filtration step. The presence of aggregates is detected by using specific antibodies.

In a preferred embodiment of the present invention, determining the amount of insoluble huntingtin is performed by using light scattering or size exclusion chromatography. In another preferred embodiment of the present invention prior to step (d) the cells are treated with an ionic detergent. In yet another preferred embodiment of the methods of the present invention, the huntingtin aggregates are filtered or transferred onto a membrane.

The present invention also relates to a method for identifying compounds affecting, e.g. interfering or enhancing the interaction of huntingtin or of a direct or indirect interaction partner of huntingtin comprising (a) contacting interacting proteins selected from the group of interacting proteins listed in table 6 in the presence or absence of a potential modulator of interaction; and (b) identifying compounds capable of modulating said interaction. The contacting is performed under conditions that permit the interaction of the two proteins. Sometimes more than two interacting proteins might be present in a single reaction as additional interaction partners of those listed under table 6, can be tested. However, the compound may also be a small molecule. Preferably said compounds are antibodies directed to huntingtin or to said interaction partner listed in table 6, wherein these antibodies are capable of interfering with the interaction with huntingtin. Alternatively, said compound is a peptide fragment of 10 to 25 amino acid residues of an interaction partner listed in table 7, wherein said peptide fragment is capable of interfering with the interaction with huntingtin. In a more preferred embodiment of the present invention, said antibody is an antibody directed to GIT1. In another more preferred embodiment of the invention, said peptide fragment is a peptide fragment of GIT1 of 10 to 25 capable of interfering with the interaction of GIT1 with huntingtin. Said interfering peptide may contain additional modifications in order to increase cellular uptake, solubility or to increase stability. Such modifications are known to the person skilled in the art and need not be listed here in detail. In a preferred embodiment of the present invention, the methods for identifying a compound further comprise the steps of modeling said compound by peptidomentics and chemically synthesizing the modeled compound.

In another preferred embodiment of the present invention, the methods for identifying a compound further comprise producing said compound. In yet another preferred embodiment of the present invention, the method for identifying said compound further comprise modifiying to achieve (i) modified site of action, spectrum of activity, organ specificity, and/or (ii) improved potency, and/or (iii) decreased toxicity (improved therapeutic index), and/or (iv) decreased side effects, and/or (v) modified onset of therapeutic action, duration of effect, and/or (vi) modified pharmakinetic parameters (resorption, distribution, metabolism and excretion), and/or (vii) modified physico-chemical parameters (solubility, hygroscopicity, color, taste, odor, stability, state), and/or (viii) improved general specificity, organ/tissue specificity, and/or (ix) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetates, or (xiv) N-acetylation to amides, phenylcarbamates, or (xv) synthesis of Mannich bases, imines, or transformation of ketones or aldehydes to Schiff's bases, oximes, acetates, ketales, enolesters, oxazolidines, thiozolidines or combinations thereof.

The present invention also relates to a method of diagnosing Huntington's disease in a biological sample comprising the steps of (a) contacting the sample with an antibody specific for a protein of table 6 or 7 or an antibody specific for the protein complex of the present invention; and (b) detecting binding of the antibody to a protein complex, wherein the detection of binding is indicative of Huntington's disease or of a predisposition to develop Huntington's disease. Preferably, binding is detected by measuring the presence of a fluorescent label bound to the protein complex.

In a preferred embodiment of the present invention's method protein complex contains (a) GIT1 or (b) said antibody is specific for a protein complex containing GIT1.

In a preferred embodiment of the present invention, said protein complex contains (a) at least one protein selected from htt, HIP15 or HP28 or (b) said antibody is specific for a protein complex containing at least one protein selected from htt, HIP15 or HP28.

The present invention also relates to a diagnostic agent/composition comprising the nucleic acid molecule of the present invention, the (poly)peptide of the present invention including/or the (poly)peptide mentioned in table 6 or 7, the antibody of the present invention, an antibody specifically reacting with a protein selected from table 7 and/or a protein selected from table 7.

Moreover, the present invention also relates to a pharmaceutical composition comprising the nucleic acid molecule of the present invention, the (poly)peptide of the present invention, the interfering compound identified with a method of the present invention, the antibody of the present invention, an antibody specifically reacting with a protein selected from table 7 and/or a protein selected from table 7.

The pharmaceutical composition will be formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations.

As a general proposition, the total pharmaceutically effective amount of pharmaceutical composition administered parenterally per dose will be in the range of about 1 μg protein/kg/day to 10 mg protein/kg/day of patient body weight, although, as noted above, this will be subject to therapeutic discretion. More preferably, this dose is at least 0.01 mg protein/kg/day, and most preferably for humans between about 0.01 and 1 mg protein/kg/day for the peptide. If given continuously, the pharmaceutical composition is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect.

Pharmaceutical compositions of the invention may be administered orally, rectally, parenterally, intracistemally, intravaginally, intraperitoneally, topically (as by powders, ointments, drops or transdermal patch), bucally, or as an oral or nasal spray. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The pharmaceutical composition is also suitably administered by sustained-release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly (2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained-release pharmaceutical composition also include liposomally entrapped protein, antibody, (poly)peptide, peptide or nucleic acid. Liposomes containing the pharmaceutical composition are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, in one embodiment, the pharmaceutical composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation preferably does not include oxidizing agents and other compounds that are known to be deleterious to (poly)peptides.

Generally, the formulations are prepared by contacting the components of the pharmaceutical composition uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non-aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG. The proteinacous components of the pharmaceutical composition are typically formulated in such vehicles at a concentration of about 0.1 mg/ml to 100 mg/ml, preferably 1-10 mg/ml, at a pH of about 3 to 8. It will be understood that the use of certain of the foregoing excipients, carriers, or stabilizers will result in the formation protein or (poly)peptide salts.

The components of the pharmaceutical composition to be used for therapeutic administration must be sterile. Sterility is readily accomplished by filtration through sterile filtration membranes (e.g., 0.2 micron membranes). Therapeutic components of the pharmaceutical composition (poly)peptide compositions generally are placed into a container having a sterile access port, for example, an intravenous solution bag or vial having a stopper pierceable by a hypodermic injection needle.

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous protein solution, and the resulting mixture is lyophilized. The infusion solution is prepared by reconstituting the lyophilized protein using bacteriostatic Water-for-Injection.

The invention also provides a pharmaceutical/diagnostic pack or kit comprising one or more containers filled with one or more of the ingredients of the pharmaceutical/diagnostic compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the (poly)peptides of the components of the pharmaceutical composition invention may be employed in conjunction with other therapeutic compounds.

Finally, the present invention relates to the use of the nucleic acid molecule of the present invention, the interfering compound identified with a method of the present invention, the (poly)peptide of the present invention including/or the (poly)peptide mentioned in table 6 or 7, the antibody of the present invention, an antibody specifically reacting with a protein selected from table 7 and/or a protein selected from table 7 for the preparation of a pharmaceutical composition for the treatment of Huntington's disease. Tables:

TABLE 1
PROTEIN-PROTEIN INTERACTIONS IN
THE PPI OF HUNTINGTIN

| Baits (DBD) | Preys (AD) |
|---|---|
| BARD1 | PLIP |
| EF1G | EF1G |
| HD1.7 | CA150 |
| HD1.7 | HIP1 |
| HD1.7 | HYPA |
| HD1.7 | SH3GL3 |
| HDexQ20 | CA150 |
| HDexQ20 | HYPA |
| HDexQ20 | SH3GL3 |
| HDexQ51 | CA150 |
| HDexQ51 | HYPA |
| HDexQ51 | SH3GL3 |
| mp53 | p53 |
| mp53 | PIASy |
| PIASy | SUMO-2 |
| PIASy | SUMO-3 |
| VIM | NEFL |
| VIM | VIMc |
| BARD1 | BAIP1 |
| BARD1 | BAIP2 |
| BARD1 | BAIP3 |
| BARD1 | FEZ1 |
| BARD1 | GIT1 |
| BARD1 | HBO1 |
| BARD1 | HIP5 |
| BARD1 | HZFH |
| BARD1 | IKAP |
| BARD1 | mHAP1 |
| BARD1 | NAG4 |
| BARD1 | PIASy |
| BARD1 | PTN |
| BARD1 | SETBD1 |
| BARD1 | ZHX1 |
| CLH-17 | Ku70 |
| CLK1 | PIASy |
| GADD45G | BAIP3 |
| GADD45G | CGI-125 |
| GADD45G | CGI-74 |
| GADD45G | EF1A |
| GADD45G | EF1G |
| GADD45G | G45IP1 |
| GADD45G | G45IP2 |
| GADD45G | G45IP3 |
| GADD45G | HIP16 |
| GADD45G | HIP5 |
| GADD45G | LUC7B1 |
| GADD45G | PIASy |
| GADD45G | PLIP |
| GADD45G | PTN |
| GADD45G | PTPK |
| hADA3 | BAIP1 |
| hADA3 | Ku70 |
| hADA3 | MAGEH1 |
| hADA3 | PIASy |
| HD1.7 | CGI-125 |
| HD1.7 | DRP-1 |
| HD1.7 | FEZ1 |
| HD1.7 | GIT1 |
| HD1.7 | HIP11 |
| HD1.7 | HIP13 |
| HD1.7 | HIP15 |
| HD1.7 | HIP16 |
| HD1.7 | HIP5 |
| HD1.7 | HZFH |
| HD1.7 | IKAP |
| HD1.7 | Ku70 |
| HD1.7 | PIASy |
| HDd1.0 | FEZ1 |
| HDd1.0 | GIT1 |
| HDd1.0 | IKAP |
| HDd1.3 | HZFH |
| HDd1.3 | IKAP |
| HDd1.3 | Ku70 |
| HDd1.3 | PIASy |
| HDexQ20 | CGI-125 |
| HDexQ20 | HIP13 |
| HDexQ20 | HP28 |
| HDexQ20 | PFN2 |
| HDexQ51 | CGI-125 |
| HDexQ51 | HIP13 |
| HDexQ51 | HIP15 |
| HDexQ51 | HP28 |
| HDexQ51 | PFN2 |
| HIP2 | PIASy |
| HIP5 | APP1 |
| HIP5 | BAIP1 |
| HIP5 | BAIP2 |
| HIP5 | CGI-74 |
| HIP5 | FEZ1 |
| HIP5 | GIT1 |
| HIP5 | HBO1 |
| HIP5 | HMP |
| HIP5 | KPNA2 |
| HIP5 | mHAP1 |
| HIP5 | NAG4 |
| HIP5 | PLIP |
| IMPD2 | PIASy |
| KPNB1 | PIASy |
| KPNB1 | PTN |
| mp53 | HZFH |
| mp53 | ZHX1 |
| PIASy | MAPIc3 |
| TAL1 | ZHX1 |
| TCP1G | Ku70 |
| VIM | ALEX2 |
| VIM | BAIP1 |
| VIM | DRP-1 |
| VIM | G45IP1 |
| VIM | HBO1 |
| VIM | HSPC232 |
| VIM | HZFH |
| VIM | PIASy |
| VIM | SETBD1 |
| VIM | SH3GL3 |
| ZNF33B | mHAP1 |
| ZNF33B | ZHX1 |

TABLE 2
Classification of proteins in Huntington's disease interaction network

| | | | | | | |
|---|---|---|---|---|---|---|
| ID | NAME | FUSION | ACCESSION | IDEN | aa MATCH | LOC |

Huntingtin fragments

| | | | | | | |
|---|---|---|---|---|---|---|
| HD1.7 | huntingtin | DBD | P42858 | 100 | 1-506 | N, C |
| HDd1.0 | huntingtin | DBD | P42858 | 100 | 1-320 | N, C |
| HDd1.3 | huntingtin | DBD | P42858 | 100 | 166-506 | N, C |
| HdexQ20 | huntingtin | DBD | P42858 | 96 | 1-90 | N, C |
| HdexQ51 | huntingtin | DBD | P42858 | 75 | 1-82 | N, C |

Transcriptional control and DNA maintenance

| | | | | | | |
|---|---|---|---|---|---|---|
| BARD1 | BRCA1 associated ring domain protein 1 | DBD | Q99728 | 99 | 1-379 | N |
| CA150 | putative transcription factor CA150 | AD | O14776 | 93 | 299-629 | N |
| GADD45G | growth arrest and DNA damage inducible protein GADD45 gamma | DBD | O95257 | 100 | 18-159 | N |
| hADA3 | ADA3 like protein | DBD | O75528 | 100 | 235-432 | N |
| HBO1 | histone acetyltransferase binding to ORC | AD | O95251 | 100 | 1-611 | N |
| PIASy | protein inhibitor of activated STAT protein gamma (PIASy) | AD, DBD | Q8N2W9 | 100 | 5-510 | N, C |
| HYPA | huntingtin interacting protein HYPA/FBP11 (fragment) | AD | O75400 | 100 | 8-422 | C, N |
| HZFH | zinc finger helicase HZFH | AD, DBD | Q9Y4I0 | 100 | 1830-2000 | N |
| IKAP | IKK complex associated protein | AD | O95163 | 100 | 1207-1332 | N, C |
| Ku70 | ATP dependent DNA helicase II, 70 kDa subunit | AD | P12956 | 100 | 298-608 | N |
| NAG4 | bromodomain containing protein NAG4 | AD | Q9NPI1 | 100 | 94-651 | N |
| p53 | cellular tumor antigen p53 | AD | P04637 | 100 | 1-393 | N |
| p53c | cellular tumor antigen p53 (C-terminus) | AD | P04637 | 100 | 248-393 | N |
| mp53 | cellular tumor antigen p53 (mouse) | DBD | P02340 | 100 | 73-390 | N |
| PLIP | cPLA2 interacting protein | AD | O95624 | 100 | 5-461 | N, PN |
| SETDB1 | histone-lysine N-methyltransferase, H3 lysine-9 specific 4 | AD | Q15047 | 100 | 1023-1291 | N |
| SUMO-2 | ubiquitin like protein SMT3A (SUMO-2) | AD | P55854 | 100 | 1-103 | C, N |
| SUMO-3 | ubiquitin like protein SMT3B (SUMO-3) | AD | P55855 | 100 | 1-95 | C, N |
| ZHX1 | zinc finger homeobox protein ZHX1 | AD | Q9UKY1 | 100 | 145-873 | N |
| ZNF33B | zinc finger protein 33b | DBD | Q8NDW3 | 100 | 527-778 | N |

Cellular organization and protein transport

| | | | | | | |
|---|---|---|---|---|---|---|
| APP1 | amyloid like protein 1 precursor | AD | P51693 | 100 | 243-555 | PM, EC |
| CLH-17 | clathrin heavy chain 1 | DBD | Q00610 | 100 | 1-289 | PM, V |
| HP28 | axonemal dynein light chain (hp28) | AD | Q9BQZ6 | 100 | 3-258 | CN |
| mHAP1 | huntingtin associated protein 1 (mouse) | AD | O35668 | 100 | 3-471 | C, EE |
| HIP1 | huntingtin interacting protein 1 | AD | O00291 | 100 | 245-631 | C, GN |
| HMP | mitofilin | AD | Q16891 | 100 | 212-758 | Mit |
| MAP1Ic3 | microtubule associated proteins 1A/1B light chain 3 | AD | Q9H491 | 100 | 58-170 | CN, MT |
| NEFL | light molecular weight neurofilament protein | AD | Q8IU72 | 100 | 1-543 | CN, IF |
| PFN2 | profilin II | AD | P35080 | 100 | 1-140 | CN |
| PTN | pleiotrophin precursor (exon 1 included) | AD | P21246 | 100 | 1-168 | PM, EC |
| SH3GL3 | SH3 containing GRB2 like protein 3 | AD | Q99963 | 100 | 3-347 | V |
| KPNA2 | karyopherin alpha-2 subunit | AD | P52292 | 100 | 141-529 | C, N |
| KPNB1 | karyopherin beta-1 subunit | DBD | Q14974 | 100 | 668-876 | C, N |
| VIM | vimentin | DBD | P08670 | 100 | 1-466 | CN, IF |
| VIMc | vimentin (C-terminus) | AD | P08670 | 100 | 190-466 | CN, IF |

Cell signaling and fate

| | | | | | | |
|---|---|---|---|---|---|---|
| ALEX2 | armadillo repeat protein ALEX2 | AD | O60267 | 100 | 127-632 | C, PM |
| CLK1 | protein kinase CLK1 | DBD | P49759 | 100 | 209-484 | N |
| FEZ1 | fasciculation and elongation protein zeta 1 | AD | Q99689 | 100 | 131-392 | C, PM |
| GIT1 | ARF GTPase activating protein GIT1 | AD | Q9Y2X7 | 98 | 249-761 | PM, V |
| PTPK | protein-tyrosine phosphatase kappa precursor | AD | Q15262 | 100 | 1227-1439 | PM, AJ |

Cellular metabolism

| | | | | | | |
|---|---|---|---|---|---|---|
| DRP-1 | dihydropyrimidinase related protein 1 (C-terminus) | AD | Q14194 | 100 | 345-572 | C |
| IMPD2 | inosine-5′-monophosphate dehydrogenase 2 | DBD | P12268 | 100 | 34-514 | C |
| TAL1 | transaldolase | DBD | P37837 | 100 | 3-337 | C |

Protein synthesis and turnover

| | | | | | | |
|---|---|---|---|---|---|---|
| EF1A | translation elongation factor 1 alpha 1 | AD | P04720 | 100 | 294-462 | C, MT |
| EF1G | elongation factor 1 gamma | AD, DBD | P26641 | 100 | 2-437 | C, MT |
| EF1Gc | elongation factor 1 gamma (C-terminus) | AD | P26641 | 100 | 123-437 | C, MT |
| HIP2 | ubiquitin conjugating enzyme E2-25 kDa | DBD | P27924 | 100 | 1-200 | C, N |
| TCPG | T-complex protein 1, gamma subunit | DBD | P49368 | 100 | 252-544 | C |

Uncharacterized proteins

| | | | | | | |
|---|---|---|---|---|---|---|
| BAIP1 | BARD1 interacting protein 1[similar to RIKEN cDNA 1810018M11] | AD | Q9BS30 | 100 | 1-226 | UN |
| BAIP2 | BARD1 interacting protein 2 [hypothetical protein] | AD | Q9H0I6 | 100 | 107-684 | UN |
| BAIP3 | BARD1 interacting protein 3 [hypothetical protein] | AD | Q96HT4 | 100 | 152-436 | UN |
| CGI-74 | CGI-74 protein | AD | Q9Y383 | 100 | 159-270 | UN |
| CGI-125 | CGI-125 protein | AD | Q9Y3C7 | 100 | 1-131 | UN |
| G45IP1 | GADD45G interacting protein 1[hypothatical protein] | AD | Q9H0V7 | 100 | 1-340 | UN |
| G45IP2 | GADD45G interacting protein 2 [B2 gene partial cDNA, clone B2E] | AD | Q9NYA0 | 100 | 566-926 | UN |
| G45IP3 | GADD45G interacting protein 3 [OK/SW-CL.16] | AD | Q8NI70 | 100 | 3-134 | UN |
| HIP5 | huntingtin interacting protein 5 [hypothetical protein KIAA1377] | AD, DBD | Q9P2H0 | 100 | 445-988 | N, C |
| HIP11 | huntingtin interacting protein 11 [hypothetical protein] | AD | Q96EZ9 | 100 | 176-328 | UN |
| HIP13 | huntingtin interacting protein 13 [metastasis suppressor protein] | AD | Q96RX2 | 100 | 512-755 | UN |
| HIP15 | huntingtin interacting protein 15 [similar to KIAA0443 gene product] | AD | Q96D09 | 100 | 663-838 | UN |
| HIP16 | huntingtin interacting protein 16 [similar to KIAA0266 gene product] | AD | Q9BVJ6 | 100 | 585-771 | UN |
| HSPC232 | HSPC232 | AD | Q9P0P6 | 92 | 1-319 | UN |
| LUC7B1 | putative SR protein LUC7B1 (SR + 89) | AD | Q9NQ29 | 99 | 116-371 | ER |
| MAGEH1 | melanoma associated antigen H1 | AD | Q9H213 | 100 | 1-219 | UN |

Abbreviations:
aa, amino acids;
IDEN, Identity;
LOC, localisation;
AD, activation domain;
DBD, DNA binding domain;
AJ, adherens junctions;
C, cytosol;
CN, cytoskeleton;
EC, extracellular space;
EE, early endosomes;
ER, endoplasmic reticulum;
IF, intermediate filaments;
GN, Golgi network;
Mit, mitochondria;
MT, microtubules;
N, nucleus;
PM, plasma membrane;
PN, perinuclear;
UN, unknown;
V, vesicles;
[ ], database annotation

TABLE 3
New proteins in Huntington's disease interaction network

| | | | | | | |
|---|---|---|---|---|---|---|
| ID | NAME | FUSION | ACCESSION | IDEN | aa MATCH | LOC |

Transcriptional control and DNA maintenance

| | | | | | | |
|---|---|---|---|---|---|---|
| BARD1 | BRCA1 associated ring domain protein 1 | DBD | Q99728 | 99 | 1-379 | N |
| CA150 | putative transcription factor CA150 | AD | O14776 | 93 | 299-629 | N |

Cell Signaling and fate

| | | | | | | |
|---|---|---|---|---|---|---|
| GIT1 | ARF GTPase activating protein GIT1 | AD | Q9Y2X7 | 98 | 249-761 | PM, V |
| HSPC232 | HSPC232 | AD | Q9P0P6 | 92 | 1-319 | UN |
| LUC7B1 | putative SR protein LUC7B1 (SR + 89) | AD | Q9NQ29 | 99 | 116-371 | ER |

Abbreviations:
aa, amino acids;
IDEN, identity;
LOC, localisation;
AD, activation domain;
DBD, DNA binding domain;
AJ, adherens junctions;
C, cytosol;
CN, cytoskeleton;
EC, extracellular space;
EE, early endosomes;
ER, endoplasmic reticulum;
IF, intermediate filaments;
GN, Golgi network;
Mit, mitochondria;
MT, microtubules;
N, nucleus;
PM, plasma membrane;
PN, perinuclear;
UN, unknown;
V, vesicles;
[ ], database annotation

TABLE 4
New protein-protein interactions, found

| Baits (DBD) | Preys (AD) |
|---|---|
| BARD1 | BAIP1 |
| BARD1 | BAIP2 |
| BARD1 | BAIP3 |
| BARD1 | FEZ1 |
| BARD1 | GIT1 |
| BARD1 | HBO1 |
| BARD1 | HIP5 |
| BARD1 | HZFH |
| BARD1 | IKAP |
| BARD1 | mHAP1 |
| BARD1 | NAG4 |
| BARD1 | PIASy |
| BARD1 | PTN |
| BARD1 | SETBD1 |
| BARD1 | ZHX1 |
| CLH-17 | Ku70 |
| CLK1 | PIASy |
| GADD45G | BAIP3 |
| GADD45G | CGI-125 |
| GADD45G | CGI-74 |
| GADD45G | EF1A |
| GADD45G | EF1G |
| GADD45G | G45IP1 |
| GADD45G | G45IP2 |
| GADD45G | G45IP3 |
| GADD45G | HIP16 |
| GADD45G | HIP5 |
| GADD45G | LUC7B1 |
| GADD45G | PIASy |
| GADD45G | PLIP |
| GADD45G | PTN |
| GADD45G | PTPK |
| hADA3 | BAIP1 |
| hADA3 | Ku70 |
| hADA3 | MAGEH1 |
| hADA3 | PIASy |
| HD1.7 | CGI-125 |
| HD1.7 | DRP-1 |
| HD1.7 | FEZ1 |
| HD1.7 | GIT1 |
| HD1.7 | HIP11 |
| HD1.7 | HIP13 |
| HD1.7 | HIP15 |
| HD1.7 | HIP16 |
| HD1.7 | HIP5 |
| HD1.7 | HZFH |
| HD1.7 | IKAP |
| HD1.7 | Ku70 |
| HD1.7 | PIASy |
| HDd1.0 | FEZ1 |
| HDd1.0 | GIT1 |
| HDd1.0 | IKAP |
| HDd1.3 | HZFH |
| HDd1.3 | IKAP |
| HDd1.3 | Ku70 |
| HDd1.3 | PIASy |
| HDexQ20 | CGI-125 |
| HDexQ20 | HIP13 |
| HDexQ20 | HP28 |
| HDexQ20 | PFN2 |
| HDexQ51 | CGI-125 |
| HDexQ51 | HIP13 |
| HDexQ51 | HIP15 |
| HDexQ51 | HP28 |
| HDexQ51 | PFN2 |
| HIP2 | PIASy |
| HIP5 | APP1 |
| HIP5 | BAIP1 |
| HIP5 | BAIP2 |
| HIP5 | CGI-74 |
| HIP5 | FEZ1 |
| HIP5 | GIT1 |
| HIP5 | HBO1 |
| HIP5 | HMP |
| HIP5 | KPNA2 |
| HIP5 | mHAP1 |
| HIP5 | NAG4 |
| HIP5 | PLIP |
| IMPD2 | PIASy |
| KPNB1 | PIASy |
| KPNB1 | PTN |
| mp53 | HZFH |
| mp53 | ZHX1 |
| PIASy | MAPIc3 |
| TAL1 | ZHX1 |
| TCP1G | Ku70 |
| VIM | ALEX2 |
| VIM | BAIP1 |
| VIM | DRP-1 |
| VIM | G45IP1 |
| VIM | HBO1 |
| VIM | HSPC232 |
| VIM | HZFH |
| VIM | PIASy |
| VIM | SETBD1 |
| VIM | SH3GL3 |
| ZNF33B | mHAP1 |
| ZNF33B | ZHX1 |

TABLE 5
Aarskog syndrome
Achromatopsia
Acoustic neuroma
Adrenal hyperplasia
Adrenoleukodystrophy
Agenesis of corpus callosum
Aicardi syndrome
Alagille syndrome
Albinism
Alopecia areata
Alstrom syndrome
Alpha-1-antitrypsin deficiency
Alzheimer
Ambiguous genitalia
Androgen insensitivity syndrome(s)
Anorchia
Angelman syndrome
Anopthalmia
Apert syndrome
Arthrogryposis
Ataxia
Autism
Bardet-Biedl syndrome
Basal cell carcinoma
Batten disease
Beckwith-Wiedemann syndrome
Blepharophimosis
Blind
Branchio-Oto-Renal (BOR) syndrome
Canavan
Cancer: (ataxia telangiectasia, basal cell nevus, brain/spine, breast, colon/bowel,
leukemia/lymphoma, lung, melanoma/skin, multiple endocrine neoplasia, oral,
ovarian, prostate, retinoblastoma, testicular, von Hippel-Lindau, xeroderma pigmentosa)
Cardiofaciocutaneous syndrome
Celiac sprue
Charcot-Marie-Tooth
CHARGE association
Chromosome anomalies - trisomy, deletions, inversions, duplications,
translocations 4p− (Wolf-Hirshhorn), 5 (cri-du-chat, 5p−), 6, 8p, 9 (trisomy 9, 9p−),
11 (11q, 11; 22), 13 (trisomy 13, Patau), 15, 16 (mosaic), 18 (18q−, 18p−, ring 18,
trisomy 18, tetrasomy 18p, Edwards), 21 (Down syndrome, trisomy 21), 22, X & Y
[sex chromosome anomalies, Klinefelter (XXY, other), Turner (XO, other), fragile-X,
other]
Cleft lip and/or cleft palate
Cockayne syndrome
Coffin-Lowry syndrome
Coffin-Siris syndrome
Congenital heart defects
Connective tissue conditions
Cooley anemia
Conjoined twins
Cornelia de Lange syndrome
Costello syndrome
Craniofacial conditions
Cri-du-Chat (5p−)
Cystic fibrosis
Cystinosis
Cystinuria
Dandy-Walker syndrome
Deaf/hard of hearing
Dermatological (skin) conditions
Developmental delay/mental retardation
DiGeorge syndrome
Down syndrome
DRPLA
Dubowitz syndrome
Dwarfism/short stature
Dysautonomia
Dystonia
Ectodermal dysplasia
Ehlers Danlos syndrome
Endocrine Conditions
Epidermolysis bullosa
Facial anomalies, disfigurement
Fanconi anemia
Fetal alcohol syndrome and effects
FG syndrome
Fragile-X syndrome
Friedreich ataxia
Freeman Sheldon syndrome
Galactosemia
Gardner syndrome
Gastroenterology conditions
Gaucher disease
Glycogen storage disease
Goldenhar syndrome
Gorlin syndrome
Hallerman Streiff syndrome
Hearing problems
Heart conditions
Hemochromatosis
Hemophilia
Hemoglobinopathies
Hereditary hemorrhagic telangiectasia
Hereditary spastic paraplegia
Hermansky-Pudlak syndrome
Hirschsprung anomaly
Holoprosencephaly
Huntington disease
Hydrocephalus
Ichthyosis
Immune deficiencies
Incontinentia pigmenti
Infertility
Intestinal problems
Joseph disease
Joubert syndrome
Kabuki syndrome
Kidney conditions
Klinefelter syndrome
Klippel-Feil syndrome
Klippel-Trenaunay syndrome
Langer-Giedion syndrome
Laurence-Moon-Biedl syndrome
Leber Optic Atrophy
Leigh disease
Lesch-Nyhan syndrome
Leukodystrophy [Adrenoleukodystrophy (ALD), Alexanders Disease, CADASIL
(Cerebral Autosomal Dominant Arteriopathy with Subcortical Infarcts &
Leukoencephalopathy), Canavan Disease (Spongy Degeneration),
Cerebrotendinous Xanthomatosis (CTX), Globoid Cell (Krabbes) Leukodystrophy,
Metachromatic Leukodystrophy (MLD), Ovarioleukodystrophy, Pelizaeus-
Merzbacher Disease, Refsum Disease, van der Knaap syndrome, Zellweger syndrome]
Limb anomalies [missing arm(s) or leg(s), Poland anomaly, other]
Lissencephaly [Isolated Sequence (ILS), X-Linked (XLIS), Subcortical Band
Heterotopia (SBH), Miller-Dieker syndrome (MDS), Microcephaly,
Microlissencephaly (MLIS), Norman-Roberts syndrome (NRS), With Cerebellar
Hypoplasia (LCH), Polymicrogyria (PMG), Schizencephaly (SCH), Muscle-Eye-
Brain (MEB) Disease, and Walker-Warburg syndrome (WWS), 17p13.3 deletion]
Liver conditions (biliary atresia, Alagille syndrome, alpha-1 antitrypsin, tyrosinemia,
neonatal hepatitis, Wilson disease)
Lowe syndrome
Lung/pulmonary conditions
Lymphedema
Maffucci syndrome(Ollier, multiple cartilaginous enchondromatosis)
Malignant hyperthermia
Maple syrup urine disease
Marinesco-Sjogren Syndrome
Marfan syndrome
Menke syndrome
Mental retardation/developmental delay
Metabolic conditions (carbohydrate deficient glycoprotein syndrome (CDGS),
diabetes insipidus, Fabry, galactosemia, glucose-6-phosphate dehydrogenase
(G6PD), fatty acid oxidation disorders, glutaric aciduria, hypophosphatemia,
Krabbe, lactic acidosis, lysosomal storage diseases, mannosidosis, maple syrup
urine, mitochondrial, neuro-metabolic, organic acidemias, PKU, purine, pyruvate
dehydrogenase deficiency, urea cycle conditions, vitamin D deficient rickets)
Miscarriage, stillbirth, infant death
Mitochondrial conditions (Alpers, Barth, beta-oxidation defects, carnitine deficiency,
CPEO, Kearns-Sayre, lactic acidosis, Leber optic neuropathy, Leigh, LCAD, Luft,
MCAD, MAD, glutaric aciduria, MERRF, MNGIE, NARP, Pearson, PHD, SCAD,
NADH-CoQ reductase, succinate dehydrogenase, Complex III, Complex IV, COX,
Complex V, other)
Moebius syndrome
Mucolipidosis, type IV (ML4)
Mucopolysaccharidosis (Hunter syndrome, Hurler syndrome, Maroteaux-Lamy
syndrome, Sanfilippo syndrome, Scheie syndrome, Morquio syndrome, other)
Multiple hereditary exostoses
Muscular dystrophy/atrophy (neuromuscular conditions including: Duchenne,
facioscapulohumeral, Charcot Marie Tooth, spinal muscular atrophy, other)
Myotonic dystrophy
Nager & Miller syndromes
Nail Patella syndrome
Narcolepsy
Neurologic conditions (neuro-metabolic, neurogenetics, neuromuscular, other)
Neurofibromatosis (von Recklinghausen)
Neuromuscular conditions
Niemann-Pick disease
Noonan syndrome
Opitz syndromes [Opitz-Frias, Opitz FG (Opitz-Kaveggia), Opitz-C
(Trigonocephaly)]
Organic acidemias
Osler-Weber-Rendu syndrome
Osteogenesis imperfecta
Oxalosis & hyperoxaluria
Pallister-Hall syndrome
Pallister-Killian syndrome (tetrasomy 12p, Teschler-Nicola syndrome)
Parkinson's disease
Periodic paralysis
Phenylketonuria (PKU)
Polycystic kidney disease
Popliteal pterygium syndrome
Porphyria
Prader-Willi syndrome
Progeria (Werner, Hutchinson-Gilford, Cockayne, Rothmond-Thomson syndromes)
Proteus syndrome
Prune belly syndrome
Pseudoxanthoma elasticum (PXE)
Psychiatric conditions
Refsum disease
Retinal degeneration
Retinitis pigmentosa (retinal degenerative diseases, Usher syndrome)
Retinoblastoma
Rett syndrome
Robinow syndrome
Rubinstein-Taybi syndrome
Russell-Silver syndrome
SBMA
SCA
Schizencephaly
Sex chromosome anomalies (47,XXY, 47,XXX, 45,X and variants, 47,XYY)
Shwachman syndrome
Sickle cell anemia
Skeletal dysplasia
Smith-Lemli-Opitz syndrome (RHS syndrome)
Smith-Magenis syndrome (17p−)
Sotos syndrome
Spina bifida (myelomeningocele, neural tube defects)
Spinal muscular atrophy (Werdnig-Hoffman, Kugelberg-Welander)
Stickler/Marshall syndrome
Sturge-Weber
Tay-Sachs disease/other (dysautonomia, dystonia, Gaucher, Niemann Pick,
Canavan, Bloom)
Thalassemia (Cooley anemia)
Thrombocytopenia absent radius syndrome
Tourette syndrome
Treacher Collins syndrome (craniofacial)
Trisomy (21, 18, 13, 9, other, see chromosome syndromes)
Tuberous sclerosis
Turner syndrome
Twins/triplets/multiple births
Unknown disorders
Urea cycle conditions
Usher syndrome
VATER association
Velo-cardio-facial syndrome (Shprintzen, DiGeorge, 22q deletion)
Visual impairment/blind
Von Hippel-Lindau syndrome
Waardenburg syndrome
Weaver syndrome
Werner syndrome
Williams syndrome
Wilson disease (hepatolenticular degeneration)
Xeroderma pigmentosum
Zellweger syndrome

TABLE 6
PROTEIN-PROTEIN INTERACTIONS IN THE
PROTEIN NETWORK OF HUNTINGTIN

| BAIT | PREY |
|---|---|
| SETDB1 | SUMO-3 |
| PIASy | SUMO-3 |
| HZFH | SUMO-3 |
| PIASy | HYPA |
| HZFH | HYPA |
| MAP1Ic3 | HYPA |
| ZHX1 | HYPA |
| PIASy | HZFH |
| HZFH | HZFH |
| GIT1 | HZFH |
| VIM | HZFH |
| PIASy | ZHX1 |
| HZFH | ZHX1 |
| VIM | ZHX1 |
| FEZ1 | HMP |
| HZFH | HMP |
| HMP | HMP |
| PIASy | HMP |
| HZFH | PTN |
| HIP15 | PTN |
| PIASy | PTN |
| PTN | PTN |
| FEZ1 | PTN |
| KPNA2 | G45IP3 |
| GIT1 | G45IP3 |
| BAIP1 | G45IP3 |
| FEZ1 | G45IP3 |
| SH3GL3 | G45IP3 |
| EF1A | APP1 |
| SETDB1 | APP1 |
| HIP16 | APP1 |
| GDF9 | APP1 |
| G45IP1 | APP1 |
| BAIP1 | APP1 |
| HIP5 | BAIP3 |
| GIT1 | BAIP3 |
| BAIP2 | BAIP3 |
| APP1 | BAIP3 |
| FEZ1 | BAIP3 |
| NAG4 | BAIP3 |
| SETDB1 | BAIP3 |
| HBO1 | BAIP3 |
| HIP15 | BAIP3 |
| BAIP3 | BAIP3 |
| HZFH | BAIP3 |
| PLIP | BAIP3 |
| mHAP1 | BAIP3 |
| PIASy | BAIP3 |
| HMP | BAIP3 |
| NAG4 | NEFL |
| HZFH | NEFL |
| VIM | NEFL |
| PIASy | NEFL |
| HMP | HIP5 |
| PLIP | HIP5 |
| mHAP1 | HIP5 |
| HBO1 | HIP5 |
| KPNA2 | HIP5 |
| VIM | HIP5 |
| APP1 | HIP5 |
| HIP15 | HIP5 |
| NAG4 | HIP5 |
| GIT1 | HIP5 |
| BAIP1 | HIP5 |
| FEZ1 | HIP5 |
| CGI-74 | HIP5 |
| BAIP2 | HIP5 |
| ALEX2 | ALEX2 |
| PIASy | MAGEH1 |
| KPNA2 | MAGEH1 |
| SETDB1 | CA150 |
| LUC7B1 | CA150 |
| HZFH | CA150 |
| PIASy | CA150 |
| PIASy | hADA3 |
| BAIP1 | hADA3 |
| MAGEH1 | hADA3 |
| Ku70 | hADA3 |
| GIT1 | BARD1 |
| BAIP3 | BARD1 |
| SETDB1 | BARD1 |
| CA150 | BARD1 |
| NAG4 | BARD1 |
| HIP15 | BARD1 |
| HIP5 | BARD1 |
| PTN | BARD1 |
| FEZ1 | BARD1 |
| IKAP | BARD1 |
| BAIP1 | BARD1 |
| mHAP1 | BARD1 |
| HBO1 | BARD1 |
| BAIP2 | BARD1 |
| PLIP | BARD1 |
| PIASy | BARD1 |
| HZFH | BARD1 |
| ZHX1 | BARD1 |
| SH3GL3 | HDexQ20 |
| HIP13 | HDexQ20 |
| CGI-125 | HDexQ20 |
| PFN2 | HDexQ20 |
| CA150 | HDexQ20 |
| HYPA | HDexQ20 |
| HP28 | HDexQ51 |
| HYPA | HDexQ51 |
| CA150 | HDexQ51 |
| SH3GL3 | HDexQ51 |
| HIP13 | HDexQ51 |
| HIP15 | HDexQ51 |
| PFN2 | HDexQ51 |
| CGI-125 | HDexQ51 |
| LUC7B1 | GADD45G |
| GDF9 | GADD45G |
| PTN | GADD45G |
| BAIP3 | GADD45G |
| G45IP2 | GADD45G |
| HIP16 | GADD45G |
| G45IP3 | GADD45G |
| CGI-125 | GADD45G |
| G45IP1 | GADD45G |
| HIP5 | GADD45G |
| EF1G | GADD45G |
| EF1A | GADD45G |
| PLIP | GADD45G |
| PIASy | GADD45G |
| CGI-74 | GADD45G |
| PTPK | GADD45G |
| MAP1Ic3 | PIASy |
| SUMO-2 | PIASy |
| SUMO-3 | PIASy |
| HYPA | HD1.7 |
| HIP16 | HD1.7 |
| DRP-1 | HD1.7 |
| HZFH | HD1.7 |
| SH3GL3 | HD1.7 |
| HIP13 | HD1.7 |
| CGI-125 | HD1.7 |
| CA150 | HD1.7 |
| HIP11 | HD1.7 |
| Ku70 | HD1.7 |
| HIP1 | HD1.7 |
| IKAP | HD1.7 |
| PFN2 | HD1.7 |
| FEZ1 | HD1.7 |
| GIT1 | HD1.7 |
| HIP5 | HD1.7 |
| PIASy | HD1.7 |
| GIT1 | HDd1.0 |
| IKAP | HDd1.0 |
| FEZ1 | HDd1.0 |
| PIASy | HDd1.3 |
| IKAP | HDd1.3 |
| HZFH | HDd1.3 |
| Ku70 | HDd1.3 |
| PIASy | HIP2 |
| Ku70 | CLH-17 |
| HZFH | mp53 |
| ZHX1 | mp53 |
| p53 | mp53 |
| PIASy | mp53 |
| PLIP | GAPD |
| PIASy | IMPD2 |
| EF1G | EF1G |
| HIP11 | EF1G |
| HZFH | TAL1 |
| ZHX1 | TAL1 |
| Ku70 | TCPG |
| PIASy | CLK1 |
| mHAP1 | ZNF33B |
| ZHX1 | ZNF33B |
| HZFH | KPNB1 |
| PIASy | KPNB1 |
| PTN | KPNB1 |
| ALEX2 | VIM |
| SH3GL3 | VIM |
| PIASy | VIM |
| HIP16 | VIM |
| HBO1 | VIM |
| BAIP1 | VIM |
| DRP-1 | VIM |
| G45IP1 | VIM |
| MOV34 | VIM |
| VIM | VIM |
| NEFL | VIM |
| HSPC232 | VIM |
| SETDB1 | VIM |
| HIP15 | HD1.7 |
| HP28 | HDexQ20 |

TABLE 7
Classification of proteins in Huntington's disease interaction network

| | | | | | | | |
|---|---|---|---|---|---|---|---|
| ID | NAME | FUSION | LOCUS ID | ACCESSION | IDEN | aa MATCH | LOC |

Huntingtin fragments

| | | | | | | | |
|---|---|---|---|---|---|---|---|
| HD1.7 | huntingtin | DBD | 3064 | P42858 | 100 | 1-506 | N, C |
| HDd1.0 | huntingtin | DBD | 3064 | P42858 | 100 | 1-320 | N, C |
| HDd1.3 | huntingtin | DBD | 3064 | P42858 | 100 | 166-506 | N, C |
| HDexQ20 | huntingtin | DBD | 3064 | P42858 | 96 | 1-90 | N, C |
| HDexQ51 | huntingtin | DBD | 3064 | P42858 | 75 | 1-82 | N, C |

Transcriptional control and DNA maintenance

| | | | | | | | |
|---|---|---|---|---|---|---|---|
| BARD1 | BRCA1 associated ring domain protein 1 | DBD | 580 | Q99728 | 99 | 1-379 | N |
| CA150 | putative transcription factor CA150 | AD, DBD | 10915 | O14776 | 93 | 299-629 | N |
| GADD45G | growth arrest and DNA damage inducible protein | DBD | 10912 | O95257 | 100 | 18-159 | N |
| | GADD45 gamma | | | | | | |
| hADA3 | ADA3 like protein | DBD | 10474 | O75528 | 100 | 235-432 | N |
| HBO1 | histone acetyltransferase binding to ORC | AD, DBD² | 11143 | O95251 | 100 | 1-611 | N |
| HYPA | huntingtin interacting protein HYPA/FBP11 (fragment) | AD, DBD | 55660 | O75400 | 100 | 8-422 | C, N |
| HZFH | zinc finger helicase HZFH | AD, DBD | 1107 | Q9Y4I0 | 100 | 1830-2000 | N |
| IKAP | IKK complex associated protein | AD, DBD² | 8518 | O95163 | 100 | 1207-1332 | N, C |
| Ku70 | ATP dependent DNA helicase II, 70 kDa subunit | AD, DBD¹ | 2547 | P12956 | 100 | 298-608 | N |
| NAG4 | bromodomain containing protein NAG4 | AD | 29117 | Q9NPI1 | 100 | 94-651 | N |
| PIASy | protein inhibitor of activated STAT protein gamma | AD, DBD | 51588 | Q8N2W9 | 100 | 5-510 | N, C |
| | (PIASy) | | | | | | |
| p53 | cellular tumor antigen p53 | AD | 7157 | P04637 | 100 | 1-393 | N |
| p53c | cellular tumor antigen p53 (C-terminus) | AD | 7157 | P04637 | 100 | 248-393 | N |
| mp53 | cellular tumor antigen p53 (mouse) | DBD | 7157 | P02340 | 100 | 73-390 | N |
| PLIP | cPLA2 interacting protein | AD, DBD¹ | 10524 | O95624 | 100 | 5-461 | N, pN |
| SETDB1 | histone-lysine N-methyltransferase, H3 lysine-9 | AD, DBD¹ | 9869 | Q15047 | 100 | 1023-1291 | N |
| | specific 4 | | | | | | |
| SUMO-2 | ubiquitin like protein SMT3A (SUMO-2) | AD | 6612 | P55854 | 100 | 1-103 | C, N |
| SUMO-3 | ubiquitin like protein SMT3B (SUMO-3) | AD, DBD | 6613 | P55855 | 100 | 1-95 | C, N |
| ZHX1 | zinc finger homeobox protein ZHX1 | AD, DBD | 11244 | Q9UKY1 | 100 | 145-873 | N |
| ZNF33B | zinc finger protein 33b | DBD | 7582 | Q8NDW3 | 100 | 527-778 | N |

Cellular organization and protein transport

| | | | | | | | |
|---|---|---|---|---|---|---|---|
| APP1 | amyloid like protein 1 precursor | AD, DBD | 333 | P51693 | 100 | 243-555 | PM, EC |
| CLH-17 | clathrin heavy chain 1 | DBD | 1213 | Q00610 | 100 | 1-289 | PM, V |
| HP28 | axonemal dynein light chain (hp28) | AD | 7802 | Q9BQZ6 | 100 | 3-258 | CN |
| mHAP1 | huntingtin associated protein 1 (mouse) | AD, DBD² | 9001 | O35668 | 100 | 3-471 | C, EE |
| HIP1 | huntingtin interacting protein 1 | AD, DBD² | 3092 | O00291 | 100 | 245-631 | C, GN |
| HMP | mitofilin | AD, DBD | 10989 | Q16891 | 100 | 212-758 | Mit |
| KPNA2 | karyopherin alpha-2 subunit | AD, DBD² | 3838 | P52292 | 100 | 141-529 | C, N |
| KPNB1 | karyopherin beta-1 subunit | DBD | 3837 | Q14974 | 100 | 668-876 | C, N |
| MAPIc3 | microtubule associated proteins 1A/1B light chain 3 | AD, DBD² | 84557 | Q9H491 | 100 | 58-170 | CN, MT |
| (MAP1Ic3) | | | | | | | |
| NEFL | light molecular weight neurofilament protein | AD, DBD | 4747 | Q8IU72 | 100 | 1-543 | CN, IF |
| PFN2 | profilin II | AD, DBD¹ | 5217 | P35080 | 100 | 1-139 | CN |
| PTN | pleiotrophin precursor (exon 1 included) | AD, DBD | 5764 | P21246 | 100 | 1-168 | PM, EC |
| SH3GL3 | SH3 containing GRB2 like protein 3 | AD, DBD² | 6457 | Q99963 | 100 | 3-347 | V |
| VIM | vimentin | DBD | 7431 | P08670 | 100 | 1-465 | CN, IF |
| VIMc | vimentin (C-terminus) | AD | 7431 | P08670 | 100 | 189-465 | CN, IF |

Cell signaling and fate

| | | | | | | | |
|---|---|---|---|---|---|---|---|
| ALEX2 | armadillo repeat protein ALEX2 | AD, DBD | 9823 | O60267 | 100 | 127-632 | C, PM |
| CLK1 | protein kinase CLK1 | DBD | 1195 | P49759 | 100 | 209-484 | N |
| DRP-1 | dihydropyrimidinase related protein 1 (C-terminus) | AD, DBD¹ | 1400 | Q14194 | 100 | 345-572 | C |
| FEZ1 | fasciculation and elongation protein zeta 1 | AD, DBD² | 9638 | Q99689 | 100 | 131-392 | C, PM |
| GDF9 | growth/differentiation factor 9 | AD, DBD¹ | 2661 | O60383 | 100 | 276-454 | C |
| GIT1 | ARF GTPase activating protein GIT1 (9 aa insertion | AD, DBD² | 28964 | Q9Y2X7 | 98 | 249-761 | PM, V |
| | included) | | | | | | |
| PTPK | protein-tyrosine phosphatase kappa precursor | AD, DBD¹ | 5796 | Q15262 | 100 | 1227-1439 | PM, AJ |

Cellular metabolism

| | | | | | | | |
|---|---|---|---|---|---|---|---|
| GAPD | glyceraldehyde 3-phosphate dehydrogenase | DBD | 2597 | P04406 | 100 | 116-334 | C |
| IMPD2 | inosine-5′-monophosphate dehydrogenase 2 | DBD | 3615 | P12268 | 100 | 34-514 | C |
| TAL1 | transaidolase | DBD | 6888 | P37837 | 100 | 3-337 | C |

Protein synthesis and turnover

| | | | | | | | |
|---|---|---|---|---|---|---|---|
| EF1A | translation elongation factor 1 alpha 1 | AD, DBD¹ | 1915 | P04720 | 100 | 294-462 | C, MT |
| EF1G | elongation factor 1 gamma | AD, DBD | 1937 | P26641 | 100 | 2-437 | C, MT |
| EF1Gc | elongation factor 1 gamma (C-terminus) | AD | 1937 | P26641 | 100 | 123-437 | C, MT |
| HIP2 | ubiquitin conjugating enzyme E2-25 kDa | DBD | 3093 | P27924 | 100 | 1-200 | C, N |
| MOV34 | MOV34 isolog | AD, DBD¹ | 10980 | O15387 | 95 | 1-297 | C, N |
| TCPG | T-complex protein 1, gamma subunit | DBD | 7203 | P49368 | 100 | 252-544 | C |

Uncharacterized proteins

| | | | | | | | |
|---|---|---|---|---|---|---|---|
| BAIP1 | BARD1 interacting protein 1[similar to RIKEN cDNA | AD | 84289 | Q9BS30 | 100 | 1-226 | UN |
| | 1810018M11] | | | | | | |
| BAIP2 | BARD1 interacting protein 2 [hypothetical protein] | AD | 84078 | Q9H0I6 | 100 | 107-684 | UN |
| BAIP3 | BARD1 interacting protein 3 [hypothetical protein] | AD, DBD | 55791 | Q96HT4 | 100 | 152-436 | UN |
| CGI-74 | CGI-74 protein | AD | 51631 | Q9Y383 | 100 | 159-270 | UN |
| CGI-125 | CGI-125 protein | AD | 51003 | Q9Y3C7 | 100 | 1-131 | UN |
| G45IP1 | GADD45G interacting protein 1[hypothetical protein] | AD, DBD² | 84060 | Q9H0V7 | 100 | 1-340 | UN |
| G45IP2 | GADD45G interacting protein 2 [B2 gene partial cDNA, | AD | 9842 | Q9NYA0 | 100 | 566-926 | UN |
| | clone B2E] | | | | | | |
| G45IP3 | GADD45G interacting protein 3 [OK/SW-CL.16] | AD, DBD | — | Q8NI70 | 100 | 3-134 | UN |
| HIP5 | huntingtin interacting protein 5 [hypothetical protein | AD, DBD | 57562 | Q9P2H0 | 100 | 445-988 | N, C |
| | KIAA1377] | | | | | | |
| HIP11 | huntingtin interacting protein 11 [hypothetical protein] | AD, DBD¹ | 1891 | Q96EZ9 | 100 | 176-328 | UN |
| HIP13 | huntingtin interacting protein 13 [metastasis suppressor | AD, DBD¹ | 9788 | Q96RX2 | 100 | 512-755 | UN |
| | protein] | | | | | | |
| HIP15 | huntingtin interacting protein 15 [similar to KIAA0443 | AD | 114928 | Q96D09 | 100 | 663-838 | UN |
| | gene product] | | | | | | |
| HIP16 | huntingtin interacting protein 16 [similar to KIAA0266 | AD | 10813 | Q9BVJ6 | 100 | 585-771 | UN |
| | gene product] | | | | | | |
| HSPC232 | HSPC232 | AD | 51535 | Q9P0P6 | 92 | 1-319 | UN |
| LUC7B1 | putative SR protein LUC7B1 (SR + 89) | AD | 55692 | Q9NQ29 | 99 | 116-371 | ER |
| MAGEH1 | melanoma associated antigen H1 | AD, DBD | 28986 | Q9H213 | 100 | 1-219 | UN |

Abbreviations:
aa, amino acids;
IDEN, identity;
LOC, localization;
LOCUS ID, NCBI LocusLink Identity, activation domain;
DBD, DNA binding domain;
DBD¹, DBD fusion proteins yielding no interactions;
DBD², autoactive DBD fusion proteins;
AJ, adherens junctions;
C, cytosol;
CN, cytoskeleton;
EC, extracellular space;
EE, early endosomes;
ER, endoplasmic reticulum;
IF, intermediate filaments;
GN, Golgi network;
Mit, mitochondria;
MT, microtubules;
N, nucleus;
PM, plasma membrane;
pN, perinuclear;
UN, unknown;
V, vesicles;
[ ], database annotation.

TABLE 8
New proteins in Huntington's disease interaction network

| | | | | | | |
|---|---|---|---|---|---|---|
| ID | NAME | FUSION | ACCESSION | IDEN | aa MATCH | LOC |

Transcriptional control and DNA maintenance

| | | | | | | |
|---|---|---|---|---|---|---|
| BARD1 | BRCA1 associated ring domain protein 1 | DBD | Q99728 | 99 | 1-379 | N |
| CA150 | putative transcription factor CA150 | AD | O14776 | 93 | 299-629 | N |

Protein synthesis and turnover

| | | | | | | |
|---|---|---|---|---|---|---|
| MOV34 | MOV34 isolog | AD, DBD | O15387 | 95 | 1-297 | C, N |

Cell Signaling and fate

| | | | | | | |
|---|---|---|---|---|---|---|
| GIT1 | ARF GTPase activating protein GIT1 | AD | Q9Y2X7 | 98 | 249-761 | PM, V |
| HSPC232 | HSPC232 | AD | Q9P0P6 | 92 | 1-319 | UN |
| LUC7B1 | putative SR protein LUC7B1 (SR + 89) | AD | Q9NQ29 | 99 | 116-371 | ER |

Abbreviations:
aa, amino acids;
IDEN, identity;
LOC, localisation;
AD, activation domain;
DBD, DNA binding domain;
AJ, adherens junctions;
C, cytosol;
CN, cytoskeleton;
EC, extracellular space;
EE, early endosomes;
ER, endoplasmic reticulum;
IF, intermediate filaments;
GN, Golgi network;
Mit, mitochondria;
MT, microtubules;
N, nucleus;
PM, plasma membrane;
PN, perinuclear;
UN, unknown;
V, vesicles;
[ ], database annotation

TABLE 9
New protein-protein interactions found

| BAIT | PREY |
|---|---|
| SETDB1 | SUMO-3 |
| PIASy | SUMO-3 |
| HZFH | SUMO-3 |
| PIASy | HYPA |
| HZFH | HYPA |
| MAP1Ic3 | HYPA |
| ZHX1 | HYPA |
| PIASy | HZFH |
| HZFH | HZFH |
| GIT1 | HZFH |
| VIM | HZFH |
| PIASy | ZHX1 |
| HZFH | ZHX1 |
| VIM | ZHX1 |
| FEZ1 | HMP |
| HZFH | HMP |
| HMP | HMP |
| PIASy | HMP |
| HZFH | PTN |
| HIP15 | PTN |
| PIASy | PTN |
| PTN | PTN |
| FEZ1 | PTN |
| KPNA2 | G45IP3 |
| GIT1 | G45IP3 |
| BAIP1 | G45IP3 |
| FEZ1 | G45IP3 |
| SH3GL3 | G45IP3 |
| EF1A | APP1 |
| SETDB1 | APP1 |
| HIP16 | APP1 |
| GDF9 | APP1 |
| G45IP1 | APP1 |
| BAIP1 | APP1 |
| HIP5 | BAIP3 |
| GIT1 | BAIP3 |
| BAIP2 | BAIP3 |
| APP1 | BAIP3 |
| FEZ1 | BAIP3 |
| NAG4 | BAIP3 |
| SETDB1 | BAIP3 |
| HBO1 | BAIP3 |
| HIP15 | BAIP3 |
| BAIP3 | BAIP3 |
| HZFH | BAIP3 |
| PLIP | BAIP3 |
| mHAP1 | BAIP3 |
| PIASy | BAIP3 |
| HMP | BAIP3 |
| NAG4 | NEFL |
| HZFH | NEFL |
| VIM | NEFL |
| PIASy | NEFL |
| HMP | HIP5 |
| PLIP | HIP5 |
| mHAP1 | HIP5 |
| HBO1 | HIP5 |
| KPNA2 | HIP5 |
| VIM | HIP5 |
| APP1 | HIP5 |
| HIP15 | HIP5 |
| NAG4 | HIP5 |
| GIT1 | HIP5 |
| BAIP1 | HIP5 |
| FEZ1 | HIP5 |
| CGI-74 | HIP5 |
| BAIP2 | HIP5 |
| ALEX2 | ALEX2 |
| PIASy | MAGEH1 |
| KPNA2 | MAGEH1 |
| SETDB1 | CA150 |
| LUC7B1 | CA150 |
| HZFH | CA150 |
| PIASy | CA150 |
| PIASy | hADA3 |
| BAIP1 | hADA3 |
| MAGEH1 | hADA3 |
| Ku70 | hADA3 |
| GIT1 | BARD1 |
| BAIP3 | BARD1 |
| SETDB1 | BARD1 |
| CA150 | BARD1 |
| NAG4 | BARD1 |
| HIP15 | BARD1 |
| HIP5 | BARD1 |
| PTN | BARD1 |
| FEZ1 | BARD1 |
| IKAP | BARD1 |
| BAIP1 | BARD1 |
| mHAP1 | BARD1 |
| HBO1 | BARD1 |
| BAIP2 | BARD1 |
| PLIP | BARD1 |
| PIASy | BARD1 |
| HZFH | BARD1 |
| ZHX1 | BARD1 |
| HIP13 | HDexQ20 |
| CGI-125 | HDexQ20 |
| PFN2 | HDexQ20 |
| HP28 | HDexQ51 |
| HIP13 | HDexQ51 |
| HIP15 | HDexQ51 |
| PFN2 | HDexQ51 |
| CGI-125 | HDexQ51 |
| LUC7B1 | GADD45G |
| GDF9 | GADD45G |
| PTN | GADD45G |
| BAIP3 | GADD45G |
| G45IP2 | GADD45G |
| HIP16 | GADD45G |
| G45IP3 | GADD45G |
| CGI-125 | GADD45G |
| G45IP1 | GADD45G |
| HIP5 | GADD45G |
| EF1G | GADD45G |
| EF1A | GADD45G |
| PLIP | GADD45G |
| PIASy | GADD45G |
| CGI-74 | GADD45G |
| PTPK | GADD45G |
| MAP1Ic3 | PIASy |
| SUMO-2 | PIASy |
| SUMO-3 | PIASy |
| HIP16 | HD1.7 |
| DRP-1 | HD1.7 |
| HZFH | HD1.7 |
| HIP13 | HD1.7 |
| CGI-125 | HD1.7 |
| HIP11 | HD1.7 |
| Ku70 | HD1.7 |
| IKAP | HD1.7 |
| PFN2 | HD1.7 |
| FEZ1 | HD1.7 |
| GIT1 | HD1.7 |
| HIP5 | HD1.7 |
| PIASy | HD1.7 |
| GIT1 | HDd1.0 |
| IKAP | HDd1.0 |
| FEZ1 | HDd1.0 |
| PIASy | HDd1.3 |
| IKAP | HDd1.3 |
| HZFH | HDd1.3 |
| Ku70 | HDd1.3 |
| PIASy | HIP2 |
| Ku70 | CLH-17 |
| HZFH | mp53 |
| ZHX1 | mp53 |
| p53 | mp53 |
| PIASy | mp53 |
| PLIP | GAPD |
| PIASy | IMPD2 |
| EF1G | EF1G |
| HIP11 | EF1G |
| HZFH | TAL1 |
| ZHX1 | TAL1 |
| Ku70 | TCPG |
| PIASy | CLK1 |
| mHAP1 | ZNF33B |
| ZHX1 | ZNF33B |
| HZFH | KPNB1 |
| PIASy | KPNB1 |
| PTN | KPNB1 |
| ALEX2 | VIM |
| SH3GL3 | VIM |
| PIASy | VIM |
| HIP16 | VIM |
| HZFH | VIM |
| HBO1 | VIM |
| BAIP1 | VIM |
| DRP-1 | VIM |
| G45IP1 | VIM |
| MOV34 | VIM |
| VIM | VIM |
| NEFL | VIM |
| HSPC232 | VIM |
| SETDB1 | VIM |
| HIP15 | HD1.7 |
| HP28 | HDexQ20 |

SUPPLEMENTARY TABLE 1
List of DBD proteins for 1^{st} round of Y2H library screens

| ID | NAME | ACCESSION | aa MATCH | PPIs |
|---|---|---|---|---|
| BARD1 | BRCA1 associated ring domain protein 1 | Q99728 | 1-379 | 3 |
| CLH-17 | clathrin heavy chain 1 | Q00610 | 1-289 | 1 |
| CLK1 | protein kinase CLK1 | P49759 | 209-484 | 1 |
| GADD45G | growth arrest and DNA-damage-inducible protein | O95257 | 18-159 | 6 |
| | GADD45 gamma | | | |
| hADA3 | ADA3 like protein | O75528 | 235-432 | 1 |
| HD1.7 | huntingtin | P42858 | 1-506 | 5 |
| HDd1.0 | huntingtin | P42858 | 1-320 | 1 |
| HDd1.3 | huntingtin | P42858 | 166-506 | 2 |
| HDexQ20 | huntingtin | P42858 | 1-90 | 3 |
| HDexQ51 | huntingtin | P42858 | 1-82 | 4 |
| HIP2 | ubiquitin conjugating enzyme E2-25 kDa | P27924 | 1-200 | 1 |
| IMPD2 | inosine-5′-monophosphate dehydrogenase 2 | P12268 | 34-514 | 1 |
| KPNB1 | karyopherin beta-1 subunit | Q14974 | 668-876 | 1 |
| mp53 | cellular tumor antigen p53 (mouse) | P02340 | 73-390 | 2 |
| TAL1 | transaldolase | P37837 | 3-337 | 1 |
| TCPG | T-complex protein 1, gamma subunit | P49368 | 252-544 | 1 |
| VIM | vimentin | P08670 | 1-465 | 6 |
| ZNF33B | zinc finger protein 33b | Q8NDW3 | 527-778 | 1 |
| 14-3-3 | 14-3-3 protein epsilon | P42655 | 93-255 | AA |
| DNAJ | DnaJ homolog subfamily A member 1 | P31689 | 113-379 | AA |
| HD513Q68 | huntingtin | P42858 | 1-513 | AA |
| HIP1 | huntingtin interacting protein 1 | O00291 | 245-631 | AA |
| mAP2A1 | α-adaptin A (mouse) | P17426 | 697-971 | AA |
| mAP2A2 | α-adaptin C (mouse) | P17427 | 697-938 | AA |
| mHAP | huntingtin associated protein 1 (mouse) | O35668 | 3-471 | AA |
| RFA | replication protein A 70 kDa DNA-binding subunit | P27694 | 262-616 | AA |
| SH3GL3 | SH3 containing GRB2 like protein 3 | Q99963 | 3-347 | AA |
| ZFR | ZNF259 | O75312 | 29-460 | AA |
| ACTG1 | gamma-actin | P02571 | 182-375 | — |
| ALBU | serum albumin precursor | P02768 | 1-249 | — |
| ALDA | fructose-bisphosphate aldolase A | P04075 | 1-363 | — |
| AMPL | cytosol aminopeptidase | P28838 | 46-487 | — |
| ARF4L | ADP-ribosylation factor-like protein 4L | P49703 | 33-201 | — |
| ASNS | glutamine-dependent asparagine synthetase | P08243 | 318-560 | — |
| BCK | creatine kinase, B chain | P12277 | 92-381 | — |
| CLH-17 | clathrin heavy chain 1 | Q00610 | 1165-1671 | — |
| GAPDH | glyceraldehyde 3-phosphate dehydrogenase | P04406 | 1-334 | — |
| HD-CT | huntingtin | P42858 | 2721-3144 | — |
| LDHB | L-lactate dehydrogenase b chain | P07195 | 96-333 | — |
| MDHM | malate dehydrogenase, mitochondrial precursor | P40926 | 1-338 | — |
| MOV34 | MOV34 isolog | O15387 | 76-297 | — |
| NSFL1C | p97 cofactor p47 | Q9UNZ2 | 201-370 | — |
| PEBP | phosphatidylethanolamine-binding protein | P30086 | 1-186 | — |
| PHGDH | D-3-phosphoglycerate dehydrogenase | O43175 | 1-553 | — |
| PLD2 | phospholipase D2 | O14939 | 168-336 | — |
| TIP49 | 49 kDa TBP-interacting protein | Q9Y265 | 1-456 | — |
| TRFE | serotransferrin precursor | P02787 | 213-698 | — |
| TUBA1 | alpha-tubulin 1 | P05209 | 1-451 | — |
| TUBB4 | tubulin beta-4 chain | Q13509 | 113-450 | — |
| UBC1 | polyubiquitin C | Q9UEF2 | 1-685 | — |

Abbreviations:
aa, amino acids;
DBD, DNA binding domain;
PPIs, protein-protein interactions;
AA, autoactivation of reporter gene.

SUPPLEMENTARY TABLE 2
Subcloned DBD proteins for 2^{nd} round of library screens

| Prey | Reason for selection | PPIs |
|---|---|---|
| HIP5 | huntingtin interacting protein verified by in vitro binding | 8 |
| | assay | |
| PIASy | huntingtin interacting protein verified by in vitro binding | 3 |
| | assay | |
| CA150 | huntingtin interacting protein, literature verified | 1 |
| | interaction [Holbert S. et al. Proc. Natl Acad. Sci. USA | |
| | 98, 1811-1816 (2001)] | |
| EF1G | part of ternary complex with EF1A, which is found in htt | 1 |
| | aggregates [Vanwetswinkel S. et al. J | |
| | Biol.Chem.278, 43443-51 (2003)] | |
| HYPA | huntingtin interacting protein, literature verified | 1 |
| | interaction [Faber, P. W. et al. Hum. Mol. Genet.9, | |
| | 1463-1474 (1998)] | |
| FEZ1 | huntingtin interacting protein verified by in vitro binding | AA |
| | assay | |
| GIT1 | huntingtin interacting protein verified by in vitro binding | AA |
| | assay | |
| EF1A | htt aggregate-interacting protein [Mitsui K. et al. J. | — |
| | Neurosci.22, 9267-9277 (2002)] | |
| HIP1.1 | huntingtin interacting protein verified by in vitro binding | — |
| | assay | |
| NEFL | vimentin interacting protein, literature verified interaction | — |
| | [Carpenter, D. A. & lp; W. J. Cell. Sci.10, 2493-2498 | |
| | (1996)] | |
| p53 | huntingtin interacting protein, literature verified | — |
| | interaction [Steffan, J. S. et al. Proc. Natl. Acad. Sci. | |
| | USA 97, 6763-8 (2000)] | |
| PLIP | BARD1 interacting protein, literature verified interaction | — |
| | [Dechend, R. et al. Oncogene 18, 3316-3323 (1999)] | |

Abbreviations:
DBD, DNA binding domain;
PPIs, protein-protein interactions;
AA, autoactivation of reporter gene.

SUPPLEMENTARY TABLE 3
Reported interactions in Huntington's disease network

| | | |
|---|---|---|
| Protein A | Protein B | Literature |

Reported interactions, found

| | | |
|---|---|---|
| CA150 | HD1.7 | Holbert S. et al. Proc. Natl Acad. Sci. USA 98, 1811-1816 (2001). The Gln-Ala repeat transcriptional |
| | HDexQ20 | activator CA150 interacts with huntingtin: neuropathologic and genetic evidence for a role in Huntington's |
| | HDexQ51 | disease pathogenesis. |
| HYPA | HD1.7 | Faber, P. W. et al. Hum. Mol. Genet.9, 1463-1474 (1998). Huntingtin interacts with a family of WW domain |
| | HDexQ20 | proteins. |
| | HDexQ51 | |
| HIP1 | HD1.7 | Wanker, E. E. et al. Hum. Mol. Genet.3, 487-495 (1997). HIP-I: a huntingtin interacting protein isolated by the |
| | | yeast two-hybrid system. |
| SH3GL3 | HD1.7 | Slttler, A. et al. Mol. Cell4, 427-436 (1998). SH3GL3 associates with the Huntingtin exon 1 protein and |
| | HDexQ20 | promotes the formation of polygln-containing protein aggregates. |
| | HDexQ51 | |
| PIASy | mp53 | Nelson, V., Davis, G. E. & Maxwell, S. A. Apoptosis3, 221-234 (2001). A putative protein inhibitor of activated |
| | | STAT (PIASy) interacts with p53 and inhibits p53-mediated transactivation but not apoptosis. |
| p53 | mp53 | Chene, P. Oncogene20, 2611-2617 (2001). The role of tetramerization in p53 function. |
| | | Leblanc V. et al. Anal Biochem.308, 247-54 (2002). Homogeneous time-resolved fluorescence assay for |
| | | identifying p53 interactions with its protein partners, directly in a cellular extract. |
| PLIP | BARD1 | Dechend, R. et al. Oncogene18, 3316-3323 (1899). The Bcl-3 oncoprotein acts as a bridging factor between |
| | | NF-kappaB/Rel and nuclear co-regulators. |
| SUMO-2 | PIASy | Sachdev, S. et al. Genes Dev.15, 3088-3103 (2001). PIASy, a nuclear matrix-associated SUMO E3 ligase, |
| | | represses LEF1 activity by sequestration into nuclear bodies. |
| SUMO-3 | PIASy | Sachdev, S. et al. Genes Dev.15, 3088-3103 (2001). PIASy, a nuclear matrix-associated SUMO E3 ligase, |
| | | represses LEF1 activity by sequestration into nuclear bodies. |
| EF1G | EF1G | Mansilla, F. et al. Biochem. J.365, 669-676 (2002). Mapping the human translation elongation factor eEF1H |
| | | complex using the yeast two-hybrid system. |
| NEFL | VIM | Carpenter, D. A. & Ip, W. J. Cell. Sci.10, 2493-2498 (1996). Neurofilament triplet protein interactions: |
| VIMc | | evidence for the preferred formation of NF-L-containing dimers and a putative function for the end domains. |

Reported interactions, not found

| | | |
|---|---|---|
| HAP1 | HDexQ20 | Li, S. H. et al. J. Biol. Chem. 273, 19220-19227 (1998) A human HAP1 homologue. Cloning, expression, and |
| | HDexQ51 | interaction with huntingtin. |
| | | Li, S. H. et al. J. Neurosci.18, 1261-1269. (1998) Interaction of huntingtin-associated protein with dynactin |
| | | P150Glued. |
| HIP1 | CLH-17 | Henry, K. R. et al. Mol. Bio.l Cell8, 2607-2625 (2002). Scd5p and clathrin function are important for cortical |
| | | actin organization, endocytosis, and localization of sla2p in yeast. [interlogs paper] |
| | | Metzler, M. et al. J. Biol. Chem. 276, 39271-39276 (2001). HIP1 functions in clathrin-mediated endocytosis |
| | | through binding to clathrin and adaptor protein 2. |
| | | Waelter, S. et al. Hum. Mol. Genet.10, 1807-1817 (2001). The huntingtin interacting protein HIP1 is a |
| | | clathrin and alpha-adaptin-binding protein involved in receptor-mediated endocytosis. |
| p53 | HDexQ20 | Steffan, J. S. et al. Proc. Natl. Acad. Sci. USA 97, 6763-6768 (2000). The Huntington's disease protein |
| | HDexQ51 | interacts with p53 and CREB-binding protein and represses transcription. |
| p53 | hADA3 | Wang, T. et al. EMBO J.20, 6404-6413 (2001). hADA3 is required for p53 activity. |
| p53 | BARD1 | Irminger-Finger, I. et al. Mol. Cell6, 1255-1266 (2001). Identification of BARD1 as mediator between |
| | | proapoptotic stress and p53-dependent apoptosis. |
| KPNA2 | KPNB1 | Chock, Y. M. & Blobel, G. Curr. Opin. Struct. Biol.6, 703-715 (2001). Karyopherins and nuclear import. |

SUPPLEMENTARY TABLE 4
Reported huntingtin interacting proteins

| | | | |
|---|---|---|---|
| ID | NAME | LOCUS ID | PubMed ID |

Transcriptional control and DNA maintenance

| | | | |
|---|---|---|---|
| CA150 | transcription elongation regulator 1 (TCERG1) | 10915 | 11172033 |
| CREB1 | cAMP responsive element binding protein 1 | 1385 | 8643525 |
| CREBBP | CREB binding protein (Rubinstein-Taybi syndrome) | 1387 | 10823891 |
| CTBP1 | C-terminal binding protein 1 | 1487 | 11739372 |
| HYPA | formin binding protein 3 (FNBP3) | 55660 | 9700202 |
| HYPB | huntingtin interacting protein B | 29072 | 9700202 |
| HYPC | huntingtin interacting protein C | 25766 | 9700202 |
| NCOR1 | nuclear receptor co-repressor 1 | 9611 | 10441327 |
| NFKB1 | nuclear factor of kappa light polypeptide gene enhancer in | 4790 | 12379151 |
| | B-cells 1 (p105) | | |
| PQBP1 | polyglutamine binding protein 1 | 10084 | 10332029 |
| REST | RE1-silencing transcription factor | 5978 | 1288172 |
| SAP30 | sin3-associated polypeptide, 30 kDa | 8819 | 10823891; 10441327 |
| SP1 | Sp1 transcription factor | 6667 | 11988536 |
| TAF4 | TAP4 RNA polymerase II | 6874 | 11988536 |
| TBP | TATA box binding protein | 6908 | 10410676 |
| TP53 | tumor protein p53 (Li-Fraumeni syndrome) | 7157 | 10823891 |

Cellular organization and protein transport

| | | | |
|---|---|---|---|
| AP2A2 | adaptor-related protein complex 2, alpha 2 subunit | 161 | 9700202 |
| DLG4 | discs, large homolog 4 (Drosophila) (PSD95) | 1742 | 11319238 |
| HAP1 | huntingtin-associated protein 1 (neuroan 1) | 9001 | 9668110; 9454836 |
| HIP1 | huntingtin interacting protein 1 | 3092 | 9147654 |
| HIP14 | huntingtin interacting protein 14 | 23390 | 9700202; 12393793 |
| OPTN | optineurin (FIP2) | 10133 | 9700202; 11137014 |
| PACSIN1 | protein kinase C and casein kinase substrate in neurons 1 | 29993 | 12354780 |
| SH3GL3 | SH3-domain GRB2-like 3 | 6457 | 9809064 |
| SYMPK | symplekin | 8189 | 9700202 |
| TUBG1 | tubulin, gamma 1 | 7283 | 11870213 |

Cell signaling and fate

| | | | |
|---|---|---|---|
| GRAP | GRB2-related adaptor protein | 10750 | 8612237 |
| GRB2 | growth factor receptor-bound protein 2 | 2885 | 9079622 |
| ITPR1 | Inositol 1,4,5-triphosphate receptor, type 1 | 3708 | 12873381 |
| MAP3K10 | mitogen-activated protein kinase kinase kinase 10 | 4294 | 10801775 |
| PDE1A | phosphodiesterase 1A, calmodulin - dependent | 5136 | 8643525 |
| RASA1 | RAS p21 protein activator (GTPase activating protein) 1 | 5921 | 8612237; 9079622 |
| TGM2 | transglutaminase 2 | 7052 | 11442349 |
| TRIP10 | thyroid hormone receptor interactor 10 | 9322 | 12604778 |

Cellular metabolism

| | | | |
|---|---|---|---|
| CBS | cystathionine-beta-synthase | 875 | 9466992; 10434301; 10823891 |
| GAPD | glyceraldehyde-3-phosphate dehydrogenase | 2597 | 8612237 |
| TPH1 | tryptophan hydroxylase 1 | 7166 | 12354289 |

Protein synthesis and turnover

| | | | |
|---|---|---|---|
| HIP2 | huntingtin interacting protein 2 | 3093 | 8702625; 9700202 |

Uncharacterized proteins

| | | | |
|---|---|---|---|
| HYPE | huntingtin interacting protein E | 11153 | 9700202 |
| HYPK | huntingtin interacting protein HYPK | 25764 | 9700202 |
| HYPM | huntingtin interacting protein HYPM | 25763 | 9700202 |
| MAGEA3 | melanoma antigen, family A, 3 | 4102 | 9700202 |

Abbreviations:
ID, interacting protein gene symbol;
LOCUS ID, NCBI LocusLink Identity;
Pubmed ID, NCBI PubMed publication index;
Reported htt interactors are presented according to databases: MINT, HPRD, BIND; Li & Ll, Trends Genet. (2004), 20, 146-152 and Harjes & Wanker, Trends. Biochem. Sci. (2003), 28, 425-433.

SUPPLEMENTARY TABLE 15
Protein-protein interactions of the extended HD network

| Number | ID 1 | LOCUSID 1 | ID 2 | LOCUSID 2 | Reference |
|---|---|---|---|---|---|
| 1 | ABL1 | 25 | CBL | 867 | literature |
| 2 | ABL1 | 25 | PXN | 5829 | literature |
| 3 | ALEX2 | 9823 | ALEX2 | 9823 | this study |
| 4 | ALK | 238 | SHC1 | 6464 | literature |
| 5 | AP2A2 | 161 | SHC1 | 6464 | literature |
| 6 | APP1 | 333 | EF1A | 1915 | this study |
| 7 | APP1 | 333 | BAIP1 | 84289 | this study |
| 8 | APP1 | 333 | GDF9 | 2661 | this study |
| 9 | APP1 | 333 | SETBD1 | 9869 | this study |
| 10 | APP1 | 333 | HIP16 | 10813 | this study |
| 11 | APP1 | 333 | BAIP3 | 55791 | this study |
| 12 | APP1 | 333 | HIP5 | 57562 | this study |
| 13 | APP1 | 333 | G45IP1 | 84060 | this study |
| 14 | AR | 367 | EP300 | 2033 | literature |
| 15 | AR | 367 | ESR1 | 2099 | literature |
| 16 | AR | 367 | RELA | 5970 | literature |
| 17 | AR | 367 | BRCA1 | 672 | literature |
| 18 | AR | 367 | HDAC1 | 3065 | literature |
| 19 | AR | 367 | NCOA1 | 8648 | literature |
| 20 | AR | 367 | JUN | 3725 | literature |
| 21 | AR | 367 | NCOA3 | 8202 | literature |
| 22 | AR | 367 | STAT3 | 6774 | literature |
| 23 | AR | 367 | NR3C1 | 2908 | literature |
| 24 | BAIP1 | 84289 | G45IP3 | — | this study |
| 25 | BAIP3 | 55791 | BAIP2 | 84078 | this study |
| 26 | BAIP3 | 55791 | HIP15 | 114928 | this study |
| 27 | BAIP3 | 55791 | BAIP3 | 55791 | this study |
| 28 | BAIP3 | 55791 | HIP5 | 57562 | this study |
| 29 | BARD1 | 580 | PLIP | 10524 | this study |
| 30 | BARD1 | 580 | ZHX1 | 11244 | this study |
| 31 | BARD1 | 580 | POU2F1 | 5451 | literature |
| 32 | BARD1 | 580 | BRCA1 | 672 | literature |
| 33 | BARD1 | 580 | CA150 | 10915 | this study |
| 34 | BARD1 | 580 | GIT1 | 28964 | this study |
| 35 | BARD1 | 580 | IKAP | 8518 | this study |
| 36 | BARD1 | 580 | HBO1 | 11143 | this study |
| 37 | BARD1 | 580 | CDC2 | 983 | literature |
| 38 | BARD1 | 580 | NAG4 | 29117 | this study |
| 39 | BARD1 | 580 | BAIP2 | 84078 | this study |
| 40 | BARD1 | 580 | PIASy | 51588 | this study |
| 41 | BARD1 | 580 | BAIP3 | 55791 | this study |
| 42 | BARD1 | 580 | HIP5 | 57562 | this study |
| 43 | BARD1 | 580 | SETBD1 | 9869 | this study |
| 44 | BARD1 | 580 | BCL3 | 602 | literature |
| 45 | BARD1 | 580 | HAP1 | 9001 | this study |
| 46 | BARD1 | 580 | PTN | 5764 | this study |
| 47 | BARD1 | 580 | HZFH | 1107 | this study |
| 48 | BARD1 | 580 | HIP15 | 114928 | this study |
| 49 | BARD1 | 580 | BAIP1 | 84289 | this study |
| 50 | BARD1 | 580 | FEZ1 | 9638 | this study |
| 51 | BCL3 | 602 | FYN | 2534 | literature |
| 52 | BCL3 | 602 | RXRA | 6256 | literature |
| 53 | BCL3 | 602 | JUN | 3725 | literature |
| 54 | BCL3 | 602 | SHC1 | 6464 | literature |
| 55 | BRCA1 | 672 | HDAC2 | 3066 | literature |
| 56 | BRCA1 | 672 | EP300 | 2033 | literature |
| 57 | BRCA1 | 672 | ESR1 | 2099 | literature |
| 58 | BRCA1 | 672 | CDC2 | 983 | literature |
| 59 | BRCA1 | 672 | HDAC1 | 3065 | literature |
| 60 | BRCA1 | 672 | STAT3 | 6774 | literature |
| 61 | BRCA1 | 672 | JUN | 3725 | literature |
| 62 | BRCA1 | 672 | MYC | 4609 | literature |
| 63 | BRCA1 | 672 | RBBP4 | 5928 | literature |
| 64 | BRCA1 | 672 | RELA | 5970 | literature |
| 65 | CA150 | 10915 | LUC7B1 | 55692 | this study |
| 66 | CA150 | 10915 | PIASy | 51588 | this study |
| 67 | CBL | 867 | SRC | 6714 | literature |
| 68 | CBL | 867 | VAV1 | 7409 | literature |
| 69 | CBL | 867 | SH3KBP1 | 30011 | literature |
| 70 | CBL | 867 | LAT | 27040 | literature |
| 71 | CBL | 867 | SHC1 | 6464 | literature |
| 72 | CBL | 867 | PIK3R1 | 5295 | literature |
| 73 | CBL | 867 | PLCG1 | 5335 | literature |
| 74 | CBL | 867 | FYN | 2534 | literature |
| 75 | CBL | 867 | PTK2B | 2185 | literature |
| 76 | CBL | 867 | EGFR | 1956 | literature |
| 77 | CDC2 | 983 | PCNA | 5111 | literature |
| 78 | CDC2 | 983 | FYN | 2534 | literature |
| 79 | CGI-74 | 51631 | HIP5 | 57562 | this study |
| 80 | CHUK | 1147 | IKBKB | 3551 | literature |
| 81 | CLH-17 | 1213 | HGS | 9146 | literature |
| 82 | CLH-17 | 1213 | Ku70 | 2547 | this study |
| 83 | CLK1 | 1195 | PIASy | 51588 | this study |
| 84 | CREB1 | 1385 | BRCA1 | 672 | literature |
| 85 | CREB1 | 1385 | NR3C1 | 2908 | literature |
| 86 | CREBBP | 1387 | MSX1 | 4487 | literature |
| 87 | CREBBP | 1387 | RELA | 5970 | literature |
| 88 | CREBBP | 1387 | RBBP4 | 5928 | literature |
| 89 | CREBBP | 1387 | PTMA | 5757 | literature |
| 90 | CREBBP | 1387 | PPARG | 5468 | literature |
| 91 | CREBBP | 1387 | PML | 5371 | literature |
| 92 | CREBBP | 1387 | MYOD1 | 4654 | literature |
| 93 | CREBBP | 1387 | JUN | 3725 | literature |
| 94 | CREBBP | 1387 | HNF4A | 3172 | literature |
| 95 | CREBBP | 1387 | NR3C1 | 2908 | literature |
| 96 | CREBBP | 1387 | EVI1 | 2122 | literature |
| 97 | CREBBP | 1387 | KLF5 | 688 | literature |
| 98 | CREBBP | 1387 | SRC | 6714 | literature |
| 99 | CREBBP | 1387 | BCL3 | 602 | literature |
| 100 | CREBBP | 1387 | TP53 | 7157 | literature |
| 101 | CREBBP | 1387 | BRCA1 | 672 | literature |
| 102 | CREBBP | 1387 | WT1 | 7490 | literature |
| 103 | CREBBP | 1387 | NCOA3 | 8202 | literature |
| 104 | CREBBP | 1387 | NCOA1 | 8648 | literature |
| 105 | CREBBP | 1387 | KHDRBS1 | 10657 | literature |
| 106 | CREBBP | 1387 | HIPK2 | 28996 | literature |
| 107 | CREBBP | 1387 | SREBF2 | 6721 | literature |
| 108 | CREBBP | 1387 | AR | 367 | literature |
| 109 | CTBP1 | 1487 | HDAC2 | 3066 | literature |
| 110 | CTBP1 | 1487 | ZNFN1A1 | 10320 | literature |
| 111 | CTBP1 | 1487 | HDAC1 | 3065 | literature |
| 112 | CTBP1 | 1487 | EVI1 | 2122 | literature |
| 113 | CTBP1 | 1487 | BRCA1 | 672 | literature |
| 114 | DLG4 | 1742 | HGS | 9146 | literature |
| 115 | DLG4 | 1742 | FYN | 2534 | literature |
| 116 | DLG4 | 1742 | PRKCA | 5578 | literature |
| 117 | DLG4 | 1742 | DNCL1 | 8655 | literature |
| 118 | DLG4 | 1742 | ERBB2 | 2064 | literature |
| 119 | DRP-1 | 1400 | Huntingtin | 3064 | this study |
| 120 | DRP-1 | 1400 | VIM | 7431 | this study |
| 121 | EF1A | 1915 | GADD45G | 10912 | this study |
| 122 | EF1A | 1915 | PLCG1 | 5335 | literature |
| 123 | EF1G | 1937 | EF1G | 1937 | this study |
| 124 | EF1G | 1937 | GADD45G | 10912 | this study |
| 125 | EGFR | 1956 | SRC | 6714 | literature |
| 126 | EGFR | 1956 | PTK2 | 5747 | literature |
| 127 | EGFR | 1956 | PLCG1 | 5335 | literature |
| 128 | EGFR | 1956 | PIK3R1 | 5295 | literature |
| 129 | EGFR | 1956 | ERBB2 | 2064 | literature |
| 130 | EGFR | 1956 | PDGFRB | 5159 | literature |
| 131 | EGFR | 1956 | PTK2B | 2185 | literature |
| 132 | EGFR | 1956 | ESR1 | 2099 | literature |
| 133 | EGFR | 1956 | SHC1 | 6464 | literature |
| 134 | EGFR | 1956 | SOS1 | 6654 | literature |
| 135 | EP300 | 2033 | ING1 | 3621 | literature |
| 136 | EP300 | 2033 | NCOA1 | 8648 | literature |
| 137 | EP300 | 2033 | HNF4A | 3172 | literature |
| 138 | EP300 | 2033 | MDM2 | 4193 | literature |
| 139 | EP300 | 2033 | PCNA | 5111 | literature |
| 140 | EP300 | 2033 | PTMA | 5757 | literature |
| 141 | EP300 | 2033 | RELA | 5970 | literature |
| 142 | EP300 | 2033 | STAT3 | 6774 | literature |
| 143 | EP300 | 2033 | ESR1 | 2099 | literature |
| 144 | EPOR | 2057 | KIT | 3815 | literature |
| 145 | EPOR | 2057 | SHC1 | 6464 | literature |
| 146 | EPOR | 2057 | VAV1 | 7409 | literature |
| 147 | EPOR | 2057 | PIK3R1 | 5295 | literature |
| 148 | ERBB2 | 2064 | PTK2 | 5747 | literature |
| 149 | ERBB2 | 2064 | SHC1 | 6464 | literature |
| 150 | ERBB2 | 2064 | PTK2B | 2185 | literature |
| 151 | ERBB2 | 2064 | SOS1 | 6654 | literature |
| 152 | ESR1 | 2099 | JUN | 3725 | literature |
| 153 | ESR1 | 2099 | MDM2 | 4193 | literature |
| 154 | ESR1 | 2099 | PIK3R1 | 5295 | literature |
| 155 | ESR1 | 2099 | SHC1 | 6464 | literature |
| 156 | ESR1 | 2099 | NCOA3 | 8202 | literature |
| 157 | ESR1 | 2099 | NCOA1 | 8648 | literature |
| 158 | EVI1 | 2122 | HDAC1 | 3065 | literature |
| 159 | FEZ1 | 9638 | HMP | 10989 | this study |
| 160 | FEZ1 | 9638 | BAIP3 | 55791 | this study |
| 161 | FEZ1 | 9638 | HIP5 | 57562 | this study |
| 162 | FEZ1 | 9638 | G45IP3 | — | this study |
| 163 | FGFR1 | 2260 | SHC1 | 6464 | literature |
| 164 | FYN | 2534 | VAV1 | 7409 | literature |
| 165 | FYN | 2534 | SHC1 | 6464 | literature |
| 166 | FYN | 2534 | KHDRBS1 | 10657 | literature |
| 167 | FYN | 2534 | WAS | 7454 | literature |
| 168 | FYN | 2534 | PDGFRB | 5159 | literature |
| 169 | FYN | 2534 | PIK3R1 | 5295 | literature |
| 170 | FYN | 2534 | PLCG1 | 5335 | literature |
| 171 | FYN | 2534 | PXN | 5829 | literature |
| 172 | FYN | 2534 | PTK2 | 5747 | literature |
| 173 | G45IP2 | 9842 | GADD45G | 10912 | this study |
| 174 | GADD45G | 10912 | G45IP1 | 84060 | this study |
| 175 | GADD45G | 10912 | HIP5 | 57562 | this study |
| 176 | GADD45G | 10912 | LUC7B1 | 55692 | this study |
| 177 | GADD45G | 10912 | RXRA | 6256 | literature |
| 178 | GADD45G | 10912 | BAIP3 | 55791 | this study |
| 179 | GADD45G | 10912 | PIASy | 51588 | this study |
| 180 | GADD45G | 10912 | G45IP3 | — | this study |
| 181 | GADD45G | 10912 | PPARG | 5468 | literature |
| 182 | GADD45G | 10912 | PCNA | 5111 | literature |
| 183 | GADD45G | 10912 | ESR1 | 2099 | literature |
| 184 | GADD45G | 10912 | CDC2 | 983 | literature |
| 185 | GADD45G | 10912 | CGI-125 | 51003 | this study |
| 186 | GADD45G | 10912 | CGI-74 | 51631 | this study |
| 187 | GAPD | 2597 | DNCL1 | 8655 | literature |
| 188 | GAPD | 2597 | PLIP | 10524 | this study |
| 189 | GDF9 | 2661 | GADD45G | 10912 | this study |
| 190 | GIT1 | 28964 | BAIP3 | 55791 | this study |
| 191 | GIT1 | 28964 | G45IP3 | — | this study |
| 192 | GIT1 | 28964 | HIP5 | 57562 | this study |
| 193 | GIT1 | 28964 | PXN | 5829 | literature |
| 194 | GIT1 | 28964 | PTK2 | 5747 | literature |
| 195 | GRAP | 10750 | EPOR | 2057 | literature |
| 196 | GRAP | 10750 | TNFSF6 | 356 | literature |
| 197 | GRAP | 10750 | KIT | 3815 | literature |
| 198 | GRAP | 10750 | SOS1 | 6654 | literature |
| 199 | GRAP | 10750 | LAT | 27040 | literature |
| 200 | GRB2 | 2885 | TP73L | 8626 | literature |
| 201 | GRB2 | 2885 | PLCG1 | 5335 | literature |
| 202 | GRB2 | 2885 | PTK2 | 5747 | literature |
| 203 | GRB2 | 2885 | SHC1 | 6464 | literature |
| 204 | GRB2 | 2885 | SOS1 | 6654 | literature |
| 205 | GRB2 | 2885 | LAT | 27040 | literature |
| 206 | GRB2 | 2885 | SRC | 6714 | literature |
| 207 | GRB2 | 2885 | WAS | 7454 | literature |
| 208 | GRB2 | 2885 | WASL | 8976 | literature |
| 209 | GRB2 | 2885 | KHDRBS1 | 10657 | literature |
| 210 | GRB2 | 2885 | SH3KBP1 | 30011 | literature |
| 211 | GRB2 | 2885 | PIK3R1 | 5295 | literature |
| 212 | GRB2 | 2885 | RASA1 | 5921 | literature |
| 213 | GRB2 | 2885 | VAV1 | 7409 | literature |
| 214 | GRB2 | 2885 | EGFR | 1956 | literature |
| 215 | GRB2 | 2885 | ABL1 | 25 | literature |
| 216 | GRB2 | 2885 | TNFSF6 | 356 | literature |
| 217 | GRB2 | 2885 | PDGFRB | 5159 | literature |
| 218 | GRB2 | 2885 | DNM1 | 1759 | literature |
| 219 | GRB2 | 2885 | EPOR | 2057 | literature |
| 220 | GRB2 | 2885 | ERBB2 | 2064 | literature |
| 221 | GRB2 | 2885 | PTK2B | 2185 | literature |
| 222 | GRB2 | 2885 | HRAS | 3265 | literature |
| 223 | GRB2 | 2885 | KIT | 3815 | literature |
| 224 | GRB2 | 2885 | CBL | 867 | literature |
| 225 | GRB2 | 2885 | FGFR1 | 2260 | literature |
| 226 | hADA3 | 10474 | EP300 | 2033 | literature |
| 227 | hADA3 | 10474 | TP53 | 7157 | literature |
| 228 | hADA3 | 10474 | BAIP1 | 84289 | this study |
| 229 | hADA3 | 10474 | PIASy | 51588 | this study |
| 230 | hADA3 | 10474 | MAGEH1 | 28986 | this study |
| 231 | hADA3 | 10474 | ESR1 | 2099 | literature |
| 232 | HAP1 | 9001 | BAIP3 | 55791 | this study |
| 233 | HAP1 | 9001 | HGS | 9146 | literature |
| 234 | HAP1 | 9001 | HIP5 | 57562 | this study |
| 235 | HBO1 | 11143 | MCM2 | 4171 | literature |
| 236 | HBO1 | 11143 | HIP5 | 57562 | this study |
| 237 | HBO1 | 11143 | BAIP3 | 55791 | this study |
| 238 | HBO1 | 11143 | AR | 367 | literature |
| 239 | HDAC1 | 3065 | PML | 5371 | literature |
| 240 | HDAC1 | 3065 | RELA | 5970 | literature |
| 241 | HDAC1 | 3065 | PTMA | 5757 | literature |
| 242 | HDAC1 | 3065 | PHB | 5245 | literature |
| 243 | HDAC1 | 3065 | MYOD1 | 4654 | literature |
| 244 | HDAC1 | 3065 | PCNA | 5111 | literature |
| 245 | HDAC1 | 3065 | RBBP4 | 5928 | literature |
| 246 | HDAC1 | 3065 | ING1 | 3621 | literature |
| 247 | HDAC1 | 3065 | HDAC2 | 3066 | literature |
| 248 | HDAC2 | 3066 | PTMA | 5757 | literature |
| 249 | HDAC2 | 3066 | RBBP4 | 5928 | literature |
| 250 | HIP11 | 1891 | EF1G | 1937 | this study |
| 251 | HIP11 | 1891 | Huntingtin | 3064 | this study |
| 252 | HIP16 | 10813 | GADD45G | 10912 | this study |
| 253 | HIP2 | 3093 | PIASy | 51588 | this study |
| 254 | HIP2 | 3093 | TP53 | 7157 | literature |
| 255 | HIP5 | 57562 | BAIP2 | 84078 | this study |
| 256 | HIP5 | 57562 | BAIP1 | 84289 | this study |
| 257 | HIP5 | 57562 | HIP15 | 114928 | this study |
| 258 | HMP | 10989 | PIASy | 51588 | this study |
| 259 | HMP | 10989 | HIP5 | 57562 | this study |
| 260 | HMP | 10989 | HMP | 10989 | this study |
| 261 | HMP | 10989 | BAIP3 | 55791 | this study |
| 262 | HNF4A | 3172 | NCOA3 | 8202 | literature |
| 263 | HNF4A | 3172 | SRC | 6714 | literature |
| 264 | HNF4A | 3172 | SREBF2 | 6721 | literature |
| 265 | HRAS | 3265 | SOS1 | 6654 | literature |
| 266 | HRAS | 3265 | VAV1 | 7409 | literature |
| 267 | HRAS | 3265 | PIK3R1 | 5295 | literature |
| 268 | HRAS | 3265 | MAPK8 | 5599 | literature |
| 269 | Huntingtin | 3064 | TUBG1 | 7283 | literature |
| 270 | Huntingtin | 3064 | RASA1 | 5921 | literature |
| 271 | Huntingtin | 3064 | HYPA | 55660 | this study |
| 272 | Huntingtin | 3064 | GRB2 | 2885 | literature |
| 273 | Huntingtin | 3064 | HIP1 | 3092 | this study |
| 274 | Huntingtin | 3064 | HIP2 | 3093 | literature |
| 275 | Huntingtin | 3064 | ITPR1 | 3708 | literature |
| 276 | Huntingtin | 3064 | REST | 5978 | literature |
| 277 | Huntingtin | 3064 | MAGEA3 | 4102 | literature |
| 278 | Huntingtin | 3064 | SH3GL3 | 6457 | this study |
| 279 | Huntingtin | 3064 | HAP1 | 9001 | literature |
| 280 | Huntingtin | 3064 | SYMPK | 8189 | literature |
| 281 | Huntingtin | 3064 | TBP | 6908 | literature |
| 282 | Huntingtin | 3064 | SP1 | 6667 | literature |
| 283 | Huntingtin | 3064 | NFKB1 | 4790 | literature |
| 284 | Huntingtin | 3064 | PDE1A | 5136 | literature |
| 285 | Huntingtin | 3064 | TAF4 | 6874 | literature |
| 286 | Huntingtin | 3064 | GAPD | 2597 | literature |
| 287 | Huntingtin | 3064 | TPH1 | 7166 | literature |
| 288 | Huntingtin | 3064 | TP53 | 7157 | literature |
| 289 | Huntingtin | 3064 | TGM2 | 7052 | literature |
| 290 | Huntingtin | 3064 | MAP3K10 | 4294 | literature |
| 291 | Huntingtin | 3064 | SAP30 | 8819 | literature |
| 292 | Huntingtin | 3064 | CREB1 | 1385 | literature |
| 293 | Huntingtin | 3064 | HIP15 | 114928 | this study |
| 294 | Huntingtin | 3064 | PIASy | 51588 | this study |
| 295 | Huntingtin | 3064 | CGI-125 | 51003 | this study |
| 296 | Huntingtin | 3064 | GIT1 | 28964 | this study |
| 297 | Huntingtin | 3064 | HIP16 | 10813 | this study |
| 298 | Huntingtin | 3064 | HIP13 | 9788 | this study |
| 299 | Huntingtin | 3064 | FEZ1 | 9638 | this study |
| 300 | Huntingtin | 3064 | IKAP | 8518 | this study |
| 301 | Huntingtin | 3064 | HP28 | 7802 | this study |
| 302 | Huntingtin | 3064 | PFN2 | 5217 | this study |
| 303 | Huntingtin | 3064 | HYPK | 25764 | literature |
| 304 | Huntingtin | 3064 | DLG4 | 1742 | literature |
| 305 | Huntingtin | 3064 | HYPE | 11153 | literature |
| 306 | Huntingtin | 3064 | CREBBP | 1387 | literature |
| 307 | Huntingtin | 3064 | CA150 | 10915 | this study |
| 308 | Huntingtin | 3064 | NCOR1 | 9611 | literature |
| 309 | Huntingtin | 3064 | PACSIN1 | 29993 | literature |
| 310 | Huntingtin | 3064 | HYPB | 29072 | literature |
| 311 | Huntingtin | 3064 | PQBP1 | 10084 | literature |
| 312 | Huntingtin | 3064 | CTBP1 | 1487 | literature |
| 313 | Huntingtin | 3064 | GRAP | 10750 | literature |
| 314 | Huntingtin | 3064 | TRIP10 | 9322 | literature |
| 315 | Huntingtin | 3064 | HYPC | 25766 | literature |
| 316 | Huntingtin | 3064 | HIP14 | 23390 | literature |
| 317 | Huntingtin | 3064 | HYPM | 25763 | literature |
| 318 | Huntingtin | 3064 | AP2A2 | 161 | literature |
| 319 | Huntingtin | 3064 | CBS | 875 | literature |
| 320 | Huntingtin | 3064 | OPTN | 10133 | literature |
| 321 | HYPA | 55660 | MAP1Ic3 | 84557 | this study |
| 322 | HZFH | 1107 | SUMO-3 | 6613 | this study |
| 323 | HZFH | 1107 | VIM | 7431 | this study |
| 324 | HZFH | 1107 | HZFH | 1107 | this study |
| 325 | HZFH | 1107 | Huntingtin | 3064 | this study |
| 326 | HZFH | 1107 | BAIP3 | 55791 | this study |
| 327 | HZFH | 1107 | HYPA | 55660 | this study |
| 328 | HZFH | 1107 | PIASy | 51588 | this study |
| 329 | HZFH | 1107 | GIT1 | 28964 | this study |
| 330 | HZFH | 1107 | ZHX1 | 11244 | this study |
| 331 | HZFH | 1107 | NEFL | 4747 | this study |
| 332 | HZFH | 1107 | CA150 | 10915 | this study |
| 333 | HZFH | 1107 | TP53 | 7157 | this study |
| 334 | HZFH | 1107 | PTN | 5764 | this study |
| 335 | HZFH | 1107 | KPNB1 | 3837 | this study |
| 336 | HZFH | 1107 | TAL1 | 6888 | this study |
| 337 | HZFH | 1107 | HMP | 10989 | this study |
| 338 | IKAP | 8518 | CHUK | 1147 | literature |
| 339 | IKAP | 8518 | IKBKB | 3551 | literature |
| 340 | IKAP | 8518 | MAPK8 | 5599 | literature |
| 341 | IMPD2 | 3615 | PIASy | 51588 | this study |
| 342 | ING1 | 3621 | PCNA | 5111 | literature |
| 343 | ING1 | 3621 | RBBP4 | 5928 | literature |
| 344 | JUN | 3725 | STAT3 | 6774 | literature |
| 345 | JUN | 3725 | RELA | 5970 | literature |
| 346 | JUN | 3725 | MYOD1 | 4654 | literature |
| 347 | JUN | 3725 | NCOA1 | 8648 | literature |
| 348 | JUN | 3725 | MAPK8 | 5599 | literature |
| 349 | KIT | 3815 | PIK3R1 | 5295 | literature |
| 350 | KIT | 3815 | PLCG1 | 5335 | literature |
| 351 | KPNA2 | 3838 | G45IP3 | — | this study |
| 352 | KPNA2 | 3838 | MAGEH1 | 28986 | this study |
| 353 | KPNA2 | 3838 | DD5 | 51366 | literature |
| 354 | KPNA2 | 3838 | RELA | 5970 | literature |
| 355 | KPNA2 | 3838 | PTMA | 5757 | literature |
| 356 | KPNA2 | 3838 | TP53 | 7157 | literature |
| 357 | KPNA2 | 3838 | HIP5 | 57562 | this study |
| 358 | KPNB1 | 3837 | TP53 | 7157 | literature |
| 359 | KPNB1 | 3837 | PIASy | 51588 | this study |
| 360 | KPNB1 | 3837 | PTN | 5764 | this study |
| 361 | KPNB1 | 3837 | DD5 | 51366 | literature |
| 362 | KPNB1 | 3837 | PTMA | 5757 | literature |
| 363 | KPNB1 | 3837 | FGFR1 | 2260 | literature |
| 364 | Ku70 | 2547 | hADA3 | 10474 | this study |
| 365 | Ku70 | 2547 | TCPG | 7203 | this study |
| 366 | Ku70 | 2547 | Huntingtin | 3064 | this study |
| 367 | Ku70 | 2547 | EGFR | 1956 | literature |
| 368 | Ku70 | 2547 | PCNA | 5111 | literature |
| 369 | Ku70 | 2547 | MAPK8 | 5599 | literature |
| 370 | Ku70 | 2547 | VAV1 | 7409 | literature |
| 371 | Ku70 | 2547 | PTTG1 | 9232 | literature |
| 372 | Ku70 | 2547 | WRN | 7486 | literature |
| 373 | Ku70 | 2547 | ABL1 | 25 | literature |
| 374 | MAGEH1 | 28986 | PIASy | 51588 | this study |
| 375 | MAP3K10 | 4294 | PHB | 5245 | literature |
| 376 | MAP3K10 | 4294 | RACGAP1 | 29127 | literature |
| 377 | MDM2 | 4193 | PML | 5371 | literature |
| 378 | MEN1 | 4221 | RELA | 5970 | literature |
| 379 | MYC | 4609 | MAPK8 | 5599 | literature |
| 380 | MYC | 4609 | RELA | 5970 | literature |
| 381 | MYOD1 | 4654 | RXRA | 6256 | literature |
| 382 | MYOD1 | 4654 | STAT3 | 6774 | literature |
| 383 | NAG4 | 29117 | HIP5 | 57562 | this study |
| 384 | NAG4 | 29117 | BAIP3 | 55791 | this study |
| 385 | NCOR1 | 9611 | PML | 5371 | literature |
| 386 | NCOR1 | 9611 | ESR1 | 2099 | literature |
| 387 | NCOR1 | 9611 | PHB | 5245 | literature |
| 388 | NCOR1 | 9611 | PTMA | 5757 | literature |
| 389 | NCOR1 | 9611 | NCOA3 | 8202 | literature |
| 390 | NCOR1 | 9611 | AR | 367 | literature |
| 391 | NCOR1 | 9611 | NR3C1 | 2908 | literature |
| 392 | NEFL | 4747 | TSC1 | 7248 | literature |
| 393 | NEFL | 4747 | PRKCL1 | 5585 | literature |
| 394 | NEFL | 4747 | PIASy | 51588 | this study |
| 395 | NEFL | 4747 | VIM | 7431 | this study |
| 396 | NEFL | 4747 | NAG4 | 29117 | this study |
| 397 | NFKB1 | 4790 | CHUK | 1147 | literature |
| 398 | NFKB1 | 4790 | AR | 367 | literature |
| 399 | NFKB1 | 4790 | KLF5 | 688 | literature |
| 400 | NFKB1 | 4790 | NR3C1 | 2908 | literature |
| 401 | NFKB1 | 4790 | MEN1 | 4221 | literature |
| 402 | NFKB1 | 4790 | IKBKB | 3551 | literature |
| 403 | NFKB1 | 4790 | BRCA1 | 672 | literature |
| 404 | NFKB1 | 4790 | STAT3 | 6774 | literature |
| 405 | NR3C1 | 2908 | NCOA1 | 8648 | literature |
| 406 | NR3C1 | 2908 | RELA | 5970 | literature |
| 407 | NR3C1 | 2908 | MDM2 | 4193 | literature |
| 408 | NR3C1 | 2908 | STAT3 | 6774 | literature |
| 409 | NR3C1 | 2908 | JUN | 3725 | literature |
| 410 | PACSIN1 | 29993 | WASL | 8976 | literature |
| 411 | PACSIN1 | 29993 | DNM1 | 1759 | literature |
| 412 | PCNA | 5111 | PTMA | 5757 | literature |
| 413 | PCNA | 5111 | WRN | 7486 | literature |
| 414 | PDGFRB | 5159 | PLCG1 | 5335 | literature |
| 415 | PDGFRB | 5159 | SHC1 | 6464 | literature |
| 416 | PDGFRB | 5159 | PIK3R1 | 5295 | literature |
| 417 | PDGFRB | 5159 | PTK2 | 5747 | literature |
| 418 | PIASy | 51588 | MAP1lc3 | 84557 | this study |
| 419 | PIASy | 51588 | BAIP3 | 55791 | this study |
| 420 | PIASy | 51588 | HYPA | 55660 | this study |
| 421 | PIK3R1 | 5295 | SHC1 | 6464 | literature |
| 422 | PIK3R1 | 5295 | SRC | 6714 | literature |
| 423 | PIK3R1 | 5295 | VAV1 | 7409 | literature |
| 424 | PIK3R1 | 5295 | WAS | 7454 | literature |
| 425 | PIK3R1 | 5295 | HGS | 9146 | literature |
| 426 | PIK3R1 | 5295 | KHDRBS1 | 10657 | literature |
| 427 | PIK3R1 | 5295 | LAT | 27040 | literature |
| 428 | PIK3R1 | 5295 | PTK2 | 5747 | literature |
| 429 | PLCG1 | 5335 | LAT | 27040 | literature |
| 430 | PLCG1 | 5335 | WAS | 7454 | literature |
| 431 | PLCG1 | 5335 | SOS1 | 6654 | literature |
| 432 | PLCG1 | 5335 | SRC | 6714 | literature |
| 433 | PLCG1 | 5335 | VAV1 | 7409 | literature |
| 434 | PLCG1 | 5335 | KHDRBS1 | 10657 | literature |
| 435 | PLIP | 10524 | BCL3 | 602 | literature |
| 436 | PLIP | 10524 | AR | 367 | literature |
| 437 | PLIP | 10524 | STAT3 | 6774 | literature |
| 438 | PLIP | 10524 | GADD45G | 10912 | this study |
| 439 | PLIP | 10524 | BAIP3 | 55791 | this study |
| 440 | PLIP | 10524 | HIP5 | 57562 | this study |
| 441 | PML | 5371 | RELA | 5970 | literature |
| 442 | PPARG | 5468 | RXRA | 6256 | literature |
| 443 | PPARG | 5468 | NCOA1 | 8648 | literature |
| 444 | PQBP1 | 10084 | AR | 367 | literature |
| 445 | PRKCA | 5578 | YWHAZ | 7534 | literature |
| 446 | PTK2 | 5747 | PXN | 5829 | literature |
| 447 | PTK2 | 5747 | SHC1 | 6464 | literature |
| 448 | PTK2 | 5747 | SRC | 6714 | literature |
| 449 | PTK2B | 2185 | SHC1 | 6464 | literature |
| 450 | PTK2B | 2185 | PIK3R1 | 5295 | literature |
| 451 | PTK2B | 2185 | PXN | 5829 | literature |
| 452 | PTK2B | 2185 | FYN | 2534 | literature |
| 453 | PTK2B | 2185 | SRC | 6714 | literature |
| 454 | PTK2B | 2185 | VAV1 | 7409 | literature |
| 455 | PTN | 5764 | GADD45G | 10912 | this study |
| 456 | PTN | 5764 | FEZ1 | 9638 | this study |
| 457 | PTN | 5764 | PTN | 5764 | this study |
| 458 | PTN | 5764 | ALK | 238 | literature |
| 459 | PTN | 5764 | PIASy | 51588 | this study |
| 460 | PTN | 5764 | HIP15 | 114928 | this study |
| 461 | PTPK | 5796 | GADD45G | 10912 | this study |
| 462 | PXN | 5829 | SRC | 6714 | literature |
| 463 | RASA1 | 5921 | PTK2B | 2185 | literature |
| 464 | RASA1 | 5921 | PIK3R1 | 5295 | literature |
| 465 | RASA1 | 5921 | PDGFRB | 5159 | literature |
| 466 | RASA1 | 5921 | HRAS | 3265 | literature |
| 467 | RASA1 | 5921 | FYN | 2534 | literature |
| 468 | RASA1 | 5921 | PXN | 5829 | literature |
| 469 | RASA1 | 5921 | ALK | 238 | literature |
| 470 | RASA1 | 5921 | SRC | 6714 | literature |
| 471 | RASA1 | 5921 | KHDRBS1 | 10657 | literature |
| 472 | RELA | 5970 | STAT3 | 6774 | literature |
| 473 | RXRA | 6256 | NCOA3 | 8202 | literature |
| 474 | SAP30 | 8819 | ING1 | 3621 | literature |
| 475 | SAP30 | 8819 | HCFC1 | 3054 | literature |
| 476 | SAP30 | 8819 | HDAC1 | 3065 | literature |
| 477 | SAP30 | 8819 | HDAC2 | 3066 | literature |
| 478 | SAP30 | 8819 | RBBP4 | 5928 | literature |
| 479 | SAP30 | 8819 | NCOR1 | 9611 | literature |
| 480 | SETBD1 | 9869 | CA150 | 10915 | this study |
| 481 | SETBD1 | 9869 | BAIP3 | 55791 | this study |
| 482 | SH3GL3 | 6457 | VIM | 7431 | this study |
| 483 | SH3GL3 | 6457 | G45IP3 | — | this study |
| 484 | SH3GL3 | 6457 | CBL | 867 | literature |
| 485 | SH3GL3 | 6457 | SH3KBP1 | 30011 | literature |
| 486 | SOS1 | 6654 | LAT | 27040 | literature |
| 487 | SOS1 | 6654 | SH3KBP1 | 30011 | literature |
| 488 | SP1 | 6667 | HNF4A | 3172 | literature |
| 489 | SP1 | 6667 | HCFC1 | 3054 | literature |
| 490 | SP1 | 6667 | BRCA1 | 672 | literature |
| 491 | SP1 | 6667 | HDAC1 | 3065 | literature |
| 492 | SP1 | 6667 | HDAC2 | 3066 | literature |
| 493 | SP1 | 6667 | JUN | 3725 | literature |
| 494 | SP1 | 6667 | MSX1 | 4487 | literature |
| 495 | SP1 | 6667 | MYC | 4609 | literature |
| 496 | SP1 | 6667 | MYOD1 | 4654 | literature |
| 497 | SP1 | 6667 | PML | 5371 | literature |
| 498 | SP1 | 6667 | POU2F1 | 5451 | literature |
| 499 | SP1 | 6667 | RBBP4 | 5928 | literature |
| 500 | SP1 | 6667 | RXRA | 6256 | literature |
| 501 | SP1 | 6667 | SHC1 | 6464 | literature |
| 502 | SP1 | 6667 | SREBF2 | 6721 | literature |
| 503 | SP1 | 6667 | KLF4 | 9314 | literature |
| 504 | SP1 | 6667 | TP53 | 7157 | literature |
| 505 | SRC | 6714 | KHDRBS1 | 10657 | literature |
| 506 | SRC | 6714 | WAS | 7454 | literature |
| 507 | SRC | 6714 | STAT3 | 6774 | literature |
| 508 | STAT3 | 6774 | NCOA1 | 8648 | literature |
| 509 | STAT3 | 6774 | KHDRBS1 | 10657 | literature |
| 510 | SUMO-2 | 6612 | PIASy | 51588 | this study |
| 511 | SUMO-3 | 6613 | PIASy | 51588 | this study |
| 512 | SUMO-3 | 6613 | PML | 5371 | literature |
| 513 | SUMO-3 | 6613 | SETBD1 | 9869 | this study |
| 514 | TAF1B | 9014 | TAF1A | 9015 | literature |
| 515 | TAF1C | 9013 | TAF1B | 9014 | literature |
| 516 | TAF1C | 9013 | TAF1A | 9015 | literature |
| 517 | TAL1 | 6888 | ZHX1 | 11244 | this study |
| 518 | TBP | 6908 | TAF1B | 9014 | literature |
| 519 | TBP | 6908 | MSX1 | 4487 | literature |
| 520 | TBP | 6908 | HMGB1 | 3146 | literature |
| 521 | TBP | 6908 | NR3C1 | 2908 | literature |
| 522 | TBP | 6908 | MCM2 | 4171 | literature |
| 523 | TBP | 6908 | MDM2 | 4193 | literature |
| 524 | TBP | 6908 | MYC | 4609 | literature |
| 525 | TBP | 6908 | RXRA | 6256 | literature |
| 526 | TBP | 6908 | NCOA3 | 8202 | literature |
| 527 | TBP | 6908 | BCL3 | 602 | literature |
| 528 | TBP | 6908 | TAF1C | 9013 | literature |
| 529 | TBP | 6908 | TP53 | 7157 | literature |
| 530 | TBP | 6908 | TAF1A | 9015 | literature |
| 531 | TBP | 6908 | ZNFN1A1 | 10320 | literature |
| 532 | TBP | 6908 | JUN | 3725 | literature |
| 533 | TBP | 6908 | NCOA1 | 8648 | literature |
| 534 | TNFSF6 | 356 | FYN | 2534 | literature |
| 535 | TNFSF6 | 356 | SRC | 6714 | literature |
| 536 | TP53 | 7157 | HMGB1 | 3146 | literature |
| 537 | TP53 | 7157 | YWHAZ | 7534 | literature |
| 538 | TP53 | 7157 | NR3C1 | 2908 | literature |
| 539 | TP53 | 7157 | HNF4A | 3172 | literature |
| 540 | TP53 | 7157 | ING1 | 3621 | literature |
| 541 | TP53 | 7157 | PIASy | 51588 | this study |
| 542 | TP53 | 7157 | PML | 5371 | literature |
| 543 | TP53 | 7157 | EP300 | 2033 | literature |
| 544 | TP53 | 7157 | MAPK8 | 5599 | literature |
| 545 | TP53 | 7157 | CHUK | 1147 | literature |
| 546 | TP53 | 7157 | WT1 | 7490 | literature |
| 547 | TP53 | 7157 | MDM2 | 4193 | literature |
| 548 | TP53 | 7157 | TP73L | 8626 | literature |
| 549 | TP53 | 7157 | TAF1C | 9013 | literature |
| 550 | TP53 | 7157 | TAF1B | 9014 | literature |
| 551 | TP53 | 7157 | TAF1A | 9015 | literature |
| 552 | TP53 | 7157 | PTTG1 | 9232 | literature |
| 553 | TP53 | 7157 | KLF4 | 9314 | literature |
| 554 | TP53 | 7157 | HIPK2 | 28996 | literature |
| 555 | TP53 | 7157 | WRN | 7486 | literature |
| 556 | TP53 | 7157 | BRCA1 | 672 | literature |
| 557 | TP53 | 7157 | ABL1 | 25 | literature |
| 558 | TP53 | 7157 | TP53 | 7157 | this study |
| 559 | TP53 | 7157 | ZHX1 | 11244 | this study |
| 560 | TP53 | 7157 | PRKCA | 5578 | literature |
| 561 | TP53 | 7157 | CDC2 | 983 | literature |
| 562 | TP73L | 8626 | HIPK2 | 28996 | literature |
| 563 | TRIP10 | 9322 | RXRA | 6256 | literature |
| 564 | TRIP10 | 9322 | WAS | 7454 | literature |
| 565 | TSC1 | 7248 | YWHAZ | 7534 | literature |
| 566 | TUBG1 | 7283 | PIK3R1 | 5295 | literature |
| 567 | TUBG1 | 7283 | BRCA1 | 672 | literature |
| 568 | TUBG1 | 7283 | PXN | 5829 | literature |
| 569 | TUBG1 | 7283 | RACGAP1 | 29127 | literature |
| 570 | VAV1 | 7409 | LAT | 27040 | literature |
| 571 | VIM | 7431 | MEN1 | 4221 | literature |
| 572 | VIM | 7431 | PRKCL1 | 5585 | literature |
| 573 | VIM | 7431 | TSC1 | 7248 | literature |
| 574 | VIM | 7431 | DNCL1 | 8655 | literature |
| 575 | VIM | 7431 | HIP16 | 10813 | this study |
| 576 | VIM | 7431 | YWHAZ | 7534 | literature |
| 577 | VIM | 7431 | VIM | 7431 | this study |
| 578 | VIM | 7431 | SETBD1 | 9869 | this study |
| 579 | VIM | 7431 | MOV34 | 10980 | this study |
| 580 | VIM | 7431 | HBO1 | 11143 | this study |
| 581 | VIM | 7431 | ZHX1 | 11244 | this study |
| 582 | VIM | 7431 | HSPC232 | 51535 | this study |
| 583 | VIM | 7431 | PIASy | 51588 | this study |
| 584 | VIM | 7431 | HIP5 | 57562 | this study |
| 585 | VIM | 7431 | G45IP1 | 84060 | this study |
| 586 | VIM | 7431 | BAIP1 | 84289 | this study |
| 587 | VIM | 7431 | ALEX2 | 9823 | this study |
| 588 | ZHX1 | 11244 | HYPA | 55660 | this study |
| 589 | ZHX1 | 11244 | PIASy | 51588 | this study |
| 590 | ZNF33B | 7558 | HAP1 | 9001 | this study |
| 591 | ZNF33B | 7558 | ZHX1 | 11244 | this study |

Abbreviations:
ID, interacting protein gene symbol;
LOCUS ID, NCBI LocusLink Identity.
The presented list of protein-protein interactions is computed from databases: MINT, HPRD, BIND; Li & Li, Trends Genet. (2004), 20, 146-152 and Harjes & Wanker, Trends. Biochem. Sci. (2003), 28, 425-433.

The figures show:

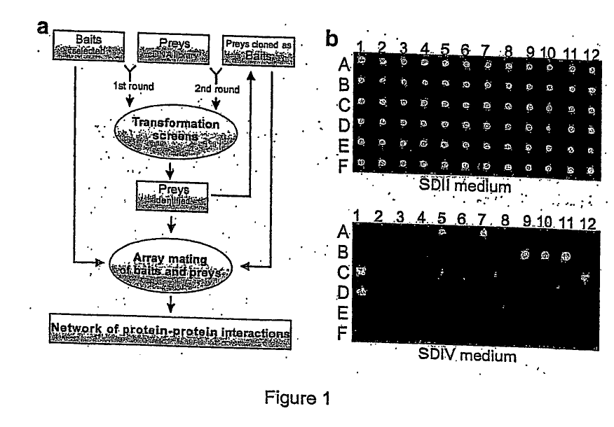

FIG. 1 Identification of two-hybrid interactions connected to HD. a, Schematic representation of the screening strategy. b, Identification of interactions by systematic interaction mating. Upper panel: Selection of diploid yeast clones by transfer on minimal medium lacking leucine and tryptophan (SDII). Lower panel: Two-hybrid selection of interactions on minimal medium lacking leucine, tryptophan, histidine and uracil (SDIV) after 5 days of growth at 30° C. The prey proteins HP28 (A5), SH3GL3 (A7), CA150 (B9), HIP15 (B10), PFN2 (B11), HIP13 (C1), CGI125 (C12) and HYPA (D1) were identified as HDexQ51 interactors.

FIG. 2 Protein interaction network for Huntington's disease. a, Matrix of 117 two-hybrid interactions between 21 bait and 49 prey proteins. b, Yeast two-hybrid interactions depicted as network using the software Pivot 1.0. In total, 96 interactions and 61 distinct proteins are depicted. In addition, dimers of EF1G, VIM and p53 are shown.

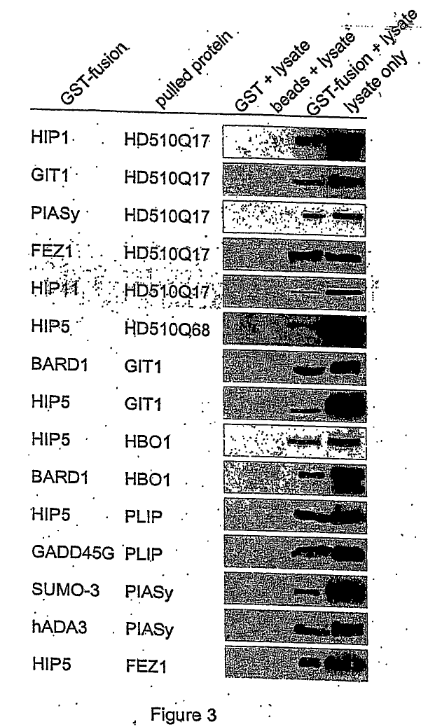

FIG. 3. Systematic validation of two-hybrid interactions by in vitro binding experiments. GST-fusion proteins (baits) immobilised on glutathione agarose beads were incubated with COS1 cell extracts containing HA-tagged prey proteins. After extensive washing of the beads, bound proteins were eluted and analysed by SDS-PAGE and immunoblotting using anti-HA antibody.

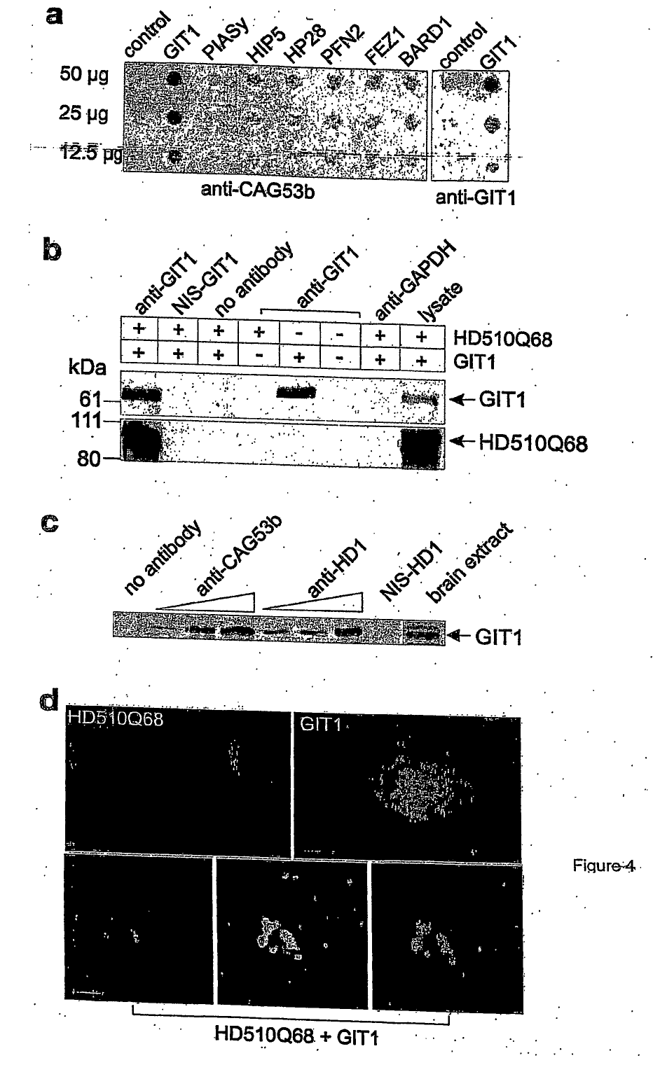

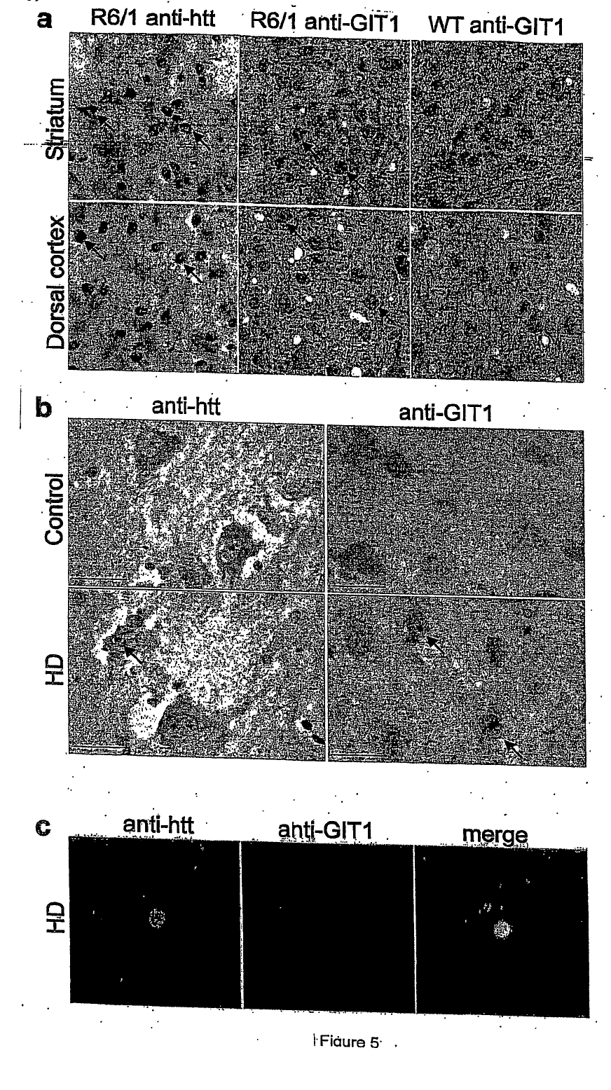

FIG. 4 Identification of network proteins stimulating htt aggregation. a, Filter retardation assay. Protein extracts were prepared from HEK293 cells coexpressing HD169Q68 and network proteins as indicated. The aggregated proteins retained on the filter were detected with anti-htt antibody (CAG53b) and anti-GIT1 antibody. b, Coimmunoprecipitation of HD510Q68 and GIT1 from COS1 cell extracts. Extracts were incubated with anti-GIT1 or preimmune serum. Immunoprecipitated material was analysed by immunoblotting using htt-antibody 4C8 and anti-HA antibody. c, Coimmunoprecipitation of htt and GIT1 from human brain extracts. Protein complexes containing GIT1 were pulled-down with increasing amounts of anti-htt antibodies, but not with corresponding preimmune sera. d, Analysis of subcellular localisation of HD510Q68 and GIT1 by immunofluorescence microscopy. COS1 cells were transfected with the indicated constructs and immunolabled with 4C8 anti-htt antibody coupled to Cy3-conjugated antibody (red) and with anti-HA antibody coupled to FITC-conjugated antibody (green). Nuclei were counterstained with Hoechst (blue). Colocalisation of HD510Q68 and GIT1 is illustrated by yellow colour of the insoluble aggregates. Scale bars, 10 μm.

FIG. 5 Detection of GIT1 in brains of R6/1 transgenic mice and HD patients. a, Sections of striatum and cortex of R6/1 mice brains labelled with anti-GIT1 and anti-htt (EM48) antisera. Arrows point to nuclear inclusions. b, Inclusions in cortex of HD patients are labelled with anti-htt (2B4) and anti-GIT1 antibodies. Arrows indicate neuronal inclusions, recognized by anti-htt (2B4) and anti-GIT1 antibodies. Scale bars, 20 μm. c, Colocalisation of GIT1 and htt in the cortex of HD patients detected by immunofluorescence microscopy.

FIG. 6 Amino acid sequence of the interacting proteins of the PPI of huntingtin.

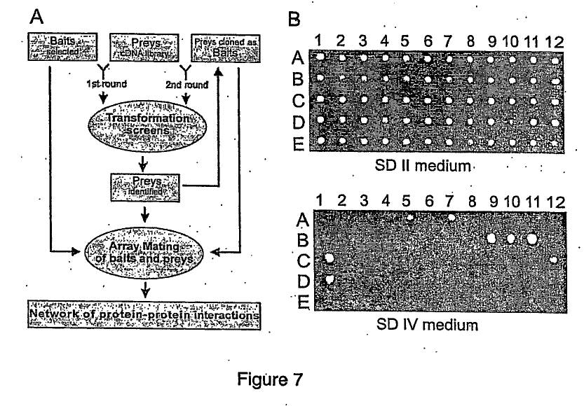

FIG. 7 Identification of Y2H interactions connected to HD. A, The screening strategy. B, Identification of interactions by systematic interaction mating. Upper panel: Selection of diploid yeast clones on SDII minimal medium. Lower panel: Two-hybrid selection of interactions on SDIV minimal medium. The prey proteins HP28 (A5), SH3GL3 (A7), CA150 (B9), HIP15 (B10), PFN2 (B11), HIP13 (C1), CGI125 (C12), and HYPA (D1) were identified as HDexQ51 interactors.

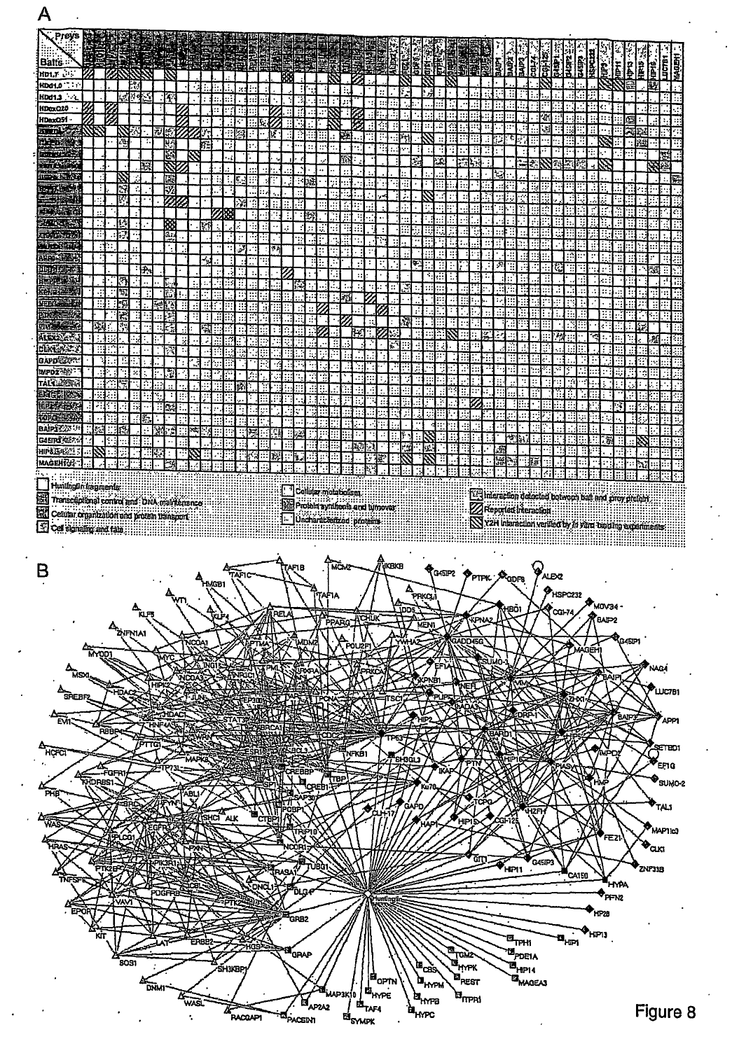

FIG. 8 A protein interaction network for Huntington's disease. A, Matrix of 186 Y2H interactions between 35 bait and 51 prey proteins. Interactions reported previously (30), or verified in pull down assays (35) are indicated. B, A comprehensive PPI network for htt. Y2H interactors identified in this study (red diamonds), previously published interactors (blue squares), interactors identified from databases HRPD, MINT and BIND, bridging any two proteins in the extended network (green triangles, Suppl. Table 5). Htt interactors previously reported and found in our screens (CA150, HYPA, HIP1, and SH3GL3), depicted as red squares.

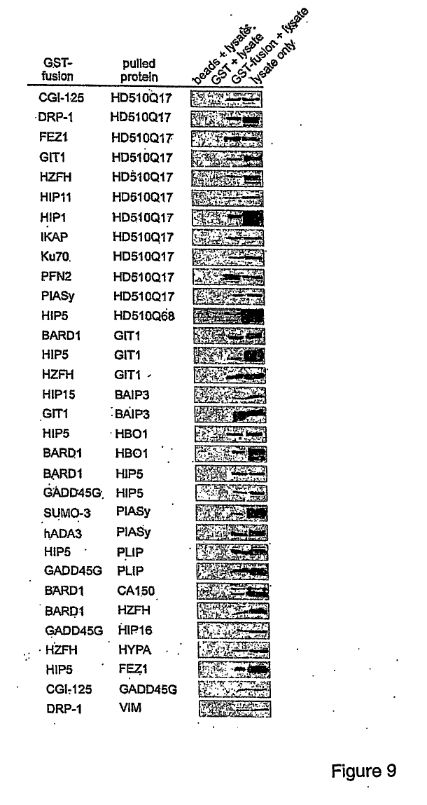

FIG. 9 Validation of Y2H interactions by in vitro binding experiments. GST-fusion proteins immobilized on glutathione agarose beads were incubated with COS-1 cell extracts containing HA-tagged proteins. After extensive washing, pulled proteins were eluted and analyzed by SDS-PAGE and immunoblotting using anti-htt 4C8 or anti-HA antibodies.

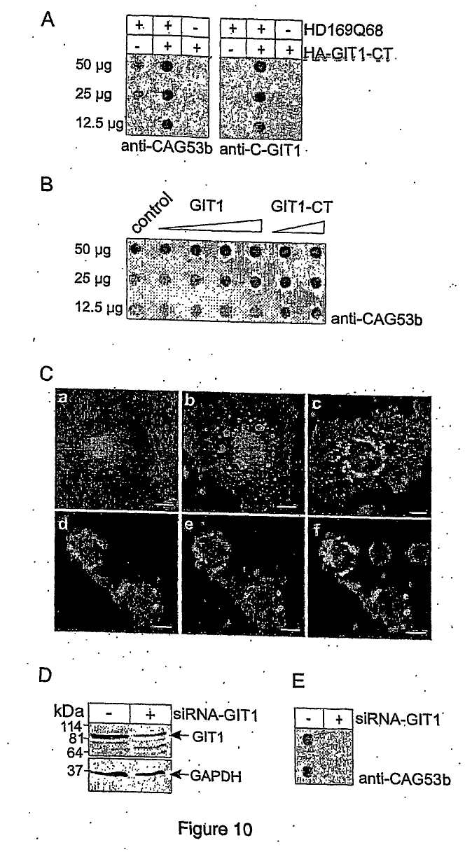

FIG. 10 GIT1 enhances and is critical for htt aggregation. A, Filter retardation assay for the identification of GIT1 as a promoter of htt aggregation. 48 h post transfection, protein extracts were prepared from HEK293 cells coexpressing HD169Q68 and GIT1-CT (aa 249-770). Aggregated proteins retained on the filter were detected with ant-htt (CAG53b) or anti-C-GIT1 antibody. B. Effect of full-length GIT1 on HD169Q68 aggregation analyzed by the filter retardation assay. C, Analysis of HD169Q68 aggregation in cells overexpressing GIT1-CT by indirect immunofluorescence microscopy. a, HD169Q68 (red). b, GIT1-CT (green). c, Colocalization of GIT1 with the endosomal marker EEAL is indicated in yellow. d-f, Colocalization of HD169Q68 (red) and GIT1-CT (green) in COS-1 cells. D, Silencing of endogenous GIT1 expression. HEK293 cells transfected with the siRNA-GIT1 were analyzed after 48 h by immunoblotting using anti-C-GIT1 and anti-GAPDH antibodies. E, Silencing of endogenous GIT1 prevents the accumulation of insoluble htt aggregates. siRNA-GIT1 treated and untreated cells expressing HD169Q68 were analyzed 72 h post transfection by filtration.

FIG. 11 Verification of the htt-GIT1 interaction. A, Coimmunoprecipitation of HD510Q68 and HA-GIT1-CT from COS-1 cell extracts using anti-C-GIT1 antibody. Immunoprecipitated material was analyzed by immunoblotting, using the anti-HA 12CA5 antibody detecting recombinant GIT1 (upper blot) and the htt-4C8 antibody (lower blot). B, Coimmunoprecipitafion of htt and GIT1 from human brain extracts. C, Subcellular localization of GIT1 and htt in differentiated PC12 cells (a-c) and SH-SY5Y cells (d-f) by confocal immunofluorescence microscopy. Colocalization of htt and GIT1 shown in yellow (panel c and f). Arrow points to cytoplasmic structures recognized by both antibodies. In addition, specific GIT1 labeling was detected at the tip of neurite-like extensions in adhesion foci (arrowheads). Scale bars, 10 μm.

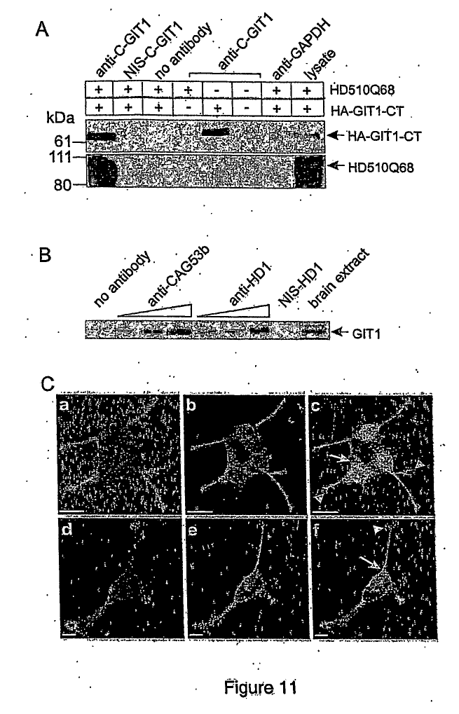

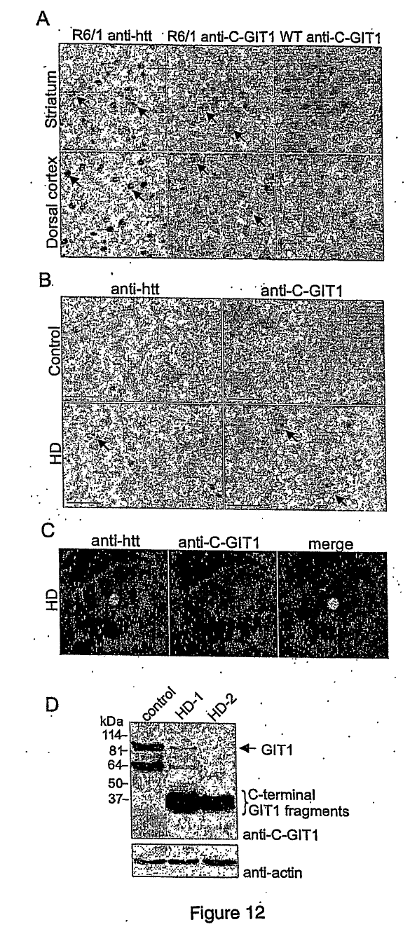

FIG. 12 Detection of GIT1 in brains of transgenic mice and HD patients. A, Sections of striatum and cortex of R6/1 mice brain labeled with anti-C-GIT1 and anti-htt EM48 antibodies. Arrows point to nuclear inclusions. B. Neuronal inclusions (arrows) in cortex of HD patients recognized by anti-htt 2B4 and anti-C-GIT1 antibodies. Scale bars, 20 μm. C, Colocalization of GIT1 and htt in the cortex of HD patients, detected by immunofluorescence microscopy. D, Detection of N-terminally truncated GIT1 degradation products in HD patient brain cortex.

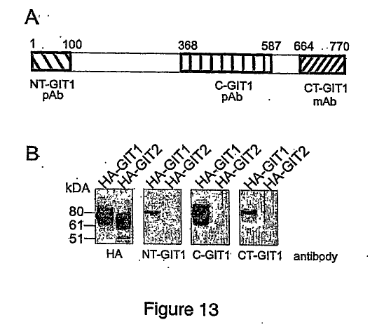

FIG. 13 Specificity of GIT1 antibodies. A, Scheme indicating the regions of GIT1, which were used for the production of antibodies. NT-GIT1 antibody recognizes the N-terminal part (aa 1-100), C-GIT1 the central part (aa 368-587) and CT-GITL the C-terminal part (aa 664-770) of GIT1. B, Analysis of the specificity of the GIT1 antibodies. All three antibodies specifically recognize GIT1, but not the homologous protein GIT2 (Premont et al., 2000). After expression of full length HAGITI and HA-GIT2 15 μg of total COS-1 cell extract was subjected to SDS-PAGE. Immunoblotting was performed with anti-NT-GIT1 (1:500), anti-C-GIT1 (1:500) and anti-CT-GIT1 (1:500) antibodies. Expression of HA-GIT1 and HA-GIT2 was detected with an anti-HA antibody (1:1000).

The examples illustrate the invention:

Part I: Establishing the Protein-Interaction Network of Huntingtin

### EXAMPLE 1

### Particular Methods and Material used in the Examples

Antibodies, Strains and Plasmids

A polyclonal antibody (pAb) against GIT1 was generated by injection of affinity purified His₆-tagged GIT1 (residues 368-587) into a rabbit. The htt-specific pAb CAG53b and HD1 were described^{13,14}. Commercially available antibodies were anti-GST pAb (Amersham Pharmacia), anti-GIT1 pAb (Santa Cruz Biotechnology), anti-HA monoclonal antibody 12CA5 (mAb) (Roche Diagnostics), anti-htt pAb EM48⁴⁷, anti-htt mAb 2B4⁴⁸ and anti-htt mAb 4C8 (Chemicon). As secondary antibodies for immunofluorescence microscopy Cy3- and FITC-conjugated IgGs (Jackson ImmunoResearch) were used. The yeast strains used as two-hybrid reporters were L40 ccua [MATa his3Δ200 trp1-901 leu2-3,112 LYS2::(lexAop)₄-HIS3 ura3::(lexAop)₈-lacZ ADE2::(lexAop)₈-URA3 GAL4 gal80can1 cyh2] and L40 ccα [MATα his3Δ200 trp1-910 leu2-3,112 ade2 LYS2::(lexAop)₄-HIS3 URA3::(lexAop)₈-lacZ GAL4 gal80 can1 cyh2]. Both strains are derivatives of L40c¹⁷. Plasmids pHD510Q17 and pHD510Q68 were generated by insertion of fragments coding for HD510Q17 and HD510Q68 into pcDNA-I (Invitrogen). pHD169Q68 was derived from pHD510Q68 by deletion of the XhoI-XhoI fragment encoding aa 170-510 of human htt.

Library Screening

Plasmids encoding bait proteins were transformed into the strain L40 ccua, tested for the absence of reporter gene activity and cotransformed with a human fetal brain cDNA library (Clontech). For each transformation 1×10⁶ independent transformants were plated onto minimal medium lacking tryptophan, leucine, histidine and uracil (SDIV medium) and incubated at 30° C. for 5 to 10 days. Clones were picked into microtitre plates using a picking robot and grown over night in liquid minimal medium lacking tryptophan and leucine (SDII medium). Then, they were spotted onto nylon or nitrocellulose membranes placed on SDIV medium plates. After incubation for 4 days membranes were subjected to a β-galactosidase (β-GAL) assay. Plasmids were prepared from positive clones and characterised by restriction analyses and sequencing. For retransformation assays plasmids encoding bait and prey proteins were cotransformed in the yeast strain L40 ccua and plated onto SDIV medium.

Array Mating Screen

Plasmids encoding bait and prey proteins were transformed into strains L40 ccua and L40 ccα, respectively. L40 ccα clones were arrayed in 96-well microtitre plates and mixed with a single L40 ccua clone for interaction mating. Diploid cells were transferred by a robot (Beckman, Biomek® 2000) onto YPD medium plates and, after incubation for 24 h at 30° C., onto SDII medium plates for additional 72 h at 36° C. For two-hybrid selection diploid cells were transferred onto SDIV medium plates with and without nylon or nitrocellulose membranes and incubated for 5 days at 30° C. The nylon or nitrocellulose membranes were subjected to the β-GAL assay. Positive clones were verified by cotransformation assays using plasmids encoding respective bait and prey proteins.

Protein Expression and Verification Assays

For verification experiments cDNA fragments encoding baits and preys were subcloned into pGEX derivatives (Stratagene) or pTL-HA¹⁸. GST fusion proteins were expressed in E. coli BL21-codon Plus™ RP (Stratagene) and affinity purified on glutathione agarose beads (Sigma) using standard protocols¹⁷. COS1 cells were transfected with mammalian expression plasmids and lysed as described¹⁸. For in vitro binding assays, 30 μg of GST or GST fusion protein were immobilized on glutathione agarose beads and incubated with 500 μg protein extract prepared from COS1 cells expressing a HA-tagged fusion protein for 2 h at 4° C. in binding buffer [50 mM HEPES pH 7.4, 150 mM NaCl, 10% glycerol, 1% NP-40, 1 mM EDTA, 20 mM NaF, 1 mM DTT, 0.1% Triton X-100, protease inhibitors (Roche Diagnostics)]. After centrifugation and extensive washing of the beads bound proteins were eluted and analysed by SDS-PAGE and Western blotting. Coimmunoprecipitation experiments were performed as described by Sittler et al.,¹⁸. For immunofluorescence microscopy COS1 cells were grown on cover slips and cotransfected with pcDNA-HD510Q68 and pTL-HA-GIT1. 40 h post transfection cells were fixed with 2% paraformaldehyde. Standard protocols for staining with appropriate primary and secondary antibodies were used¹⁸.

Filter Retardation Assay

HEK293 cells coexpressing HD169Q68 and GIT1, PIASy, HIP5, HP28, PFN2, FEZ1 or BARD1 were harvested 48 h post transfection. Cells were lysed as described 18 and boiled in 2% SDS, 100 mM DTT for 5 min. Aliquots containing 50, 25 and 12.5 μg of total protein were used for filtration on a cellulose acetate membrane 1 SDS-resistant aggregates were detected using anti-CAG53b or anti-GIT1 antibodies.

Immunocytochemistry

Mice were deeply anaesthetised and perfused through the left cardiac ventricle with 4% paraformaldehyde in 0.1 M phosphate buffer. Brains were removed and postfixed overnight in 4% paraformaldehyde. Sections were processed for immunocytochemistry as described⁴⁷. pAb EM48 (1:1000) and affinity purified anti-GIT1 pAb (1:100) were used as primary antibodies.

Six human HD and 5 control brains were used in this study. Two HD cases were classified as grade 3 and four cases as grade 4 of neuropathological severity. For immunolabelling standard protocols were used⁴⁸. 2B4 mAb (1:200) and affinity purified GIT1 pAb (1:50) were used as primary antibodies.

### EXAMPLE 2

### Two-Hybrid Screens and Data Management

To generate a PPI network for HD we used a combination of library and matrix yeast two-hybrid screens (FIG. 1a). First, 50 selected cDNAs encoding proteins potentially involved in HD including 10 different htt fragments were cloned into a DNA binding domain vector for expression of LexA fusion proteins (baits). The resulting plasmids were introduced into yeast strain L40 ccua, which carries three reporter genes, HIS3, URA3 and lacZ, for two-hybrid interaction analyses. Forty baits did not activate the reporters by themselves and were used individually for cotransformation screening of a human fetal brain cDNA library expressing GAL4 activation domain hybrids (preys). In each screen, 1×10⁶ auxotrophic transformants were tested on selective plates, and 1-50 positive colonies were typically obtained. Restriction analyses and sequencing identified preys that together with their respective baits repeatedly activated the reporter genes. Starting with 40 baits in the first round of cotransformation screens we identified 34 PPIs for 10 baits (Table 1).

In the second round of screens, 12 cDNA fragments encoding preys identified in the first screen were subcloned into a DNA binding domain vector. The resulting baits were tested for autoactivation and 10 were screened against a human fetal brain cDNA library. Four of the 10 proteins revealed additional 13 PPIs.

Finally, an array mating screen was performed to connect all baits and preys identified in the transformation screens. For this assay, MATα yeast cultures were transformed with plasmids encoding prey proteins and arrayed in 96-well microtitre plates for interaction mating with individual MATa strains expressing bait proteins. Using this strategy each bait was individually tested for interaction with every prey in the array. Diploid yeast clones, formed by mating on YPD plates, were selected on agar SDII plates, and further transferred by a spotting robot on SDIV plates to select for Y2H interactions (FIG. 1b). We examined 3500 pairwise combinations of baits and preys in the mating assay and identified additional 70 PPIs. These interactions could be confirmed in cotransformation assays (Table 5).

TABLE 5
Summary of two-hybrid screens

| | | | baits | |
|---|---|---|---|---|
| | baits | preys | yielding | interactions |
| Screen | screened | screened | interactions | identified |
| 1st transformation | 40 | 4 × 10⁷ | 10 | 34 |
| screen | | | | |
| 2nd transformation | 10 | 1 × 10⁷ | 4 | 13 |
| screen | | | | |
| Array mating screen | 50 | 70 | 21 | 70 |

Thus, the combination of cDNA library and array mating screens proved powerful in establishing a highly connected PPI network linked to htt.

Sequence analysis of the cDNAs encoding bait and prey proteins revealed ORFs ranging from 82 to 728 amino acids in size (Table 2). In a systematic Blast search 60 out of the 67 proteins identified were identical to a SwissProt or TrEMBL protein entry (http://us.expasy.org/sprot/). The remaining 7 proteins showed 75-99% identity to its best fit and either contained single amino acid substitutions, variable polyQ lengths or small regions of sequence variation. Uncharacterised proteins were named according to their interaction partners. Each ORF was further examined for consensus protein domains using the FprintScan, HMMPfam, HMMSmart, ProfileScan, and BlastProDom programs providing useful hints to protein function. For example, the protein BAIP1 (BARD1 interacting protein 1) possesses a Zn-finger-like PHD finger that is believed to be important for chromatin-mediated transcriptional regulation. Similarly, domain searches for BAIP2 (BARD1 interacting protein 2) revealed a BTB/POZ domain, a motif found in developmentally regulated zinc finger proteins of the Kelch family of actin-associated proteins. Thus, BAIP2 could potentially mediate the association of BARD1 with the actin cytoskeleton.

### EXAMPLE 3

### Analysis and Functional Assignment of the Two-Hybrid Data

Our two-hybrid screens identified a total of 117 PPIs between 70 protein fragments. As a result of the iterative two-hybrid strategy all interactions could be depicted in a single large network. The number of interactions identified for each bait varied from 1 to 18, with each protein having 1.6 interaction partners on average. In order to display the PPI data, both matrix and network representations were used (FIG. 2). The matrix shows, in addition to the two-hybrid interactions, previously reported interactions and interactions verified by independent methods (FIG. 2a). In comparison, the network view allows to immediately recognize local PPI patterns and paths connecting two proteins in the network (FIG. 2b). Interestingly, proteins such as htt, BARD1, GADD45G, HIP5, PIASy or VIM interact with more than 11 other proteins forming nodes within the HD network, while 30 proteins have only one interaction partner and thus are located at the periphery of the network (FIG. 2b). Indeed, all other proteins are embedded in many bi-fan motifs and multiple circular interaction clusters that have been interpreted to be an indication for biological relevance^{11,19}. Schwikowski et al.²⁰ defined network proteins, which are separated by no more than two other proteins, as being part of a functional cluster. In this respect all proteins in our network form a functional cluster with htt.

We assigned a subcellular localisation to each protein by examining various sources of literature and based on available experimental data we grouped the proteins into six broad functional categories (FIG. 2a, Table 2).

Eighteen proteins in the HD network are involved in transcriptional regulation or DNA maintenance (FIG. 2a). The second largest group, 14 proteins, includes mainly cytoskeletal and transport proteins. We assigned 5 proteins to cellular signalling and fate, another 4 proteins to protein synthesis and turnover, and 3 proteins to cellular metabolism. Being part of 41 interactions, 16 proteins of unknown function, were identified.

For the analysis of htt PPIs, as much as 40 out of 117 interactions (34,2%) included a htt fragment (FIG. 2a). In total, 19 different htt interacting partners from various functional groups were detected, 4 proteins had been previously described and 6 involved proteins of unknown function. Surprisingly, most htt partners (6) are involved in transcriptional regulation and DNA maintenance, but others function in cell organization and transport (4), cellular signalling (2), or cellular metabolism (1), suggesting that htt functions in different subcellular processes.

The current hypothesis that htt has a function in transcriptional regulation is inferred from, its interactions with transcriptional activators, coactivators or repressors²¹ In agreement with previous reports, binding of htt to CA150²² and HYPA²³ has been detected in our screens. In addition, new connections to nuclear proteins such as SETBD1, PLIP and HBO1 were found. These multidomain proteins act on histones and are known modulators of chromatin structure and gene expression. Similarly, the zinc finger bromo domain containing proteins BARD1, NAG4, HZFH, ZHX1, ZNF33B play a role in transcriptional control. The protein IKAP directly interacts with htt and was recently shown to be part of a complex regulating RNA polymerase II activity²⁴. Htt also interacts with PIASy, which inhibits transcription factor STAT-mediated gene activation²⁵. PIASy functions as SUMO E3 ligase for the Wnt-responsive transcription factor LEF1, inhibiting its activity via sumoylation²⁶. This suggests that PIASy catalysed sumoylation of transcription factors could represent a general mechanism in repression of gene expression. The binding of PIASy to htt indicates that htt may itself be a substrate for sumoylation. Alternatively, it could influence the sumoylation of other transcription factors. Thus, our data extend the nuclear role of htt and provide additional leads for its involvement in transcriptional regulation.

Another large group of htt interactors identified here are proteins that function in cellular organization and vesicle transport. We report a new interaction between htt and dynein light chain (HP28), a component of the dynein/dynactin motor protein complex. Interestingly, the p150^{Glued} subunit of dynactin is linked to the htt-associated proteinHAP1^{16,27}. Our observation that htt directly binds to HP28 underscores the potential scaffolding role of htt/HAP1 in dynein/dynactin driven retrograde vesicle transport along microtubules in axons.

The htt interacting protein HIP1 anchors clathrin-coated vesicles to the cytoskeleton via its actin-binding domain, a link crucial for synaptic vesicle endocytosis28. Here, a new PPI between htt and profilin II (PFN2)²⁹ was detected. PFN2, a protein enriched in neurons, modulates actin polymerization in vitro and is involved in endocytosis via association with scaffolding proteins²⁹. The htt-PFN2 connection adds support to a potential role of htt in modulation of both actin polymerization and vesicle transport processes.

Currently, for the function of 6 htt interactors, including HIP5, no genetic or biochemical evidence is available (Table 2). We found that HIP5 binds to htt as well as to karyopherin a (KPNA2). KPNA2 serves as an adapter for karyopherin β (KPNB1), which transports NLS-tagged proteins into the nucleus³⁰. Thus, HIP5 might take this route to the nucleus. Interestingly, HEAT or armadillo (ARM) repeats, forming α-helical structures in KPNA2 and KPNB1 are also present in htt³¹ Therefore, the complexes between KPNA2 and HIP5 as well as between htt and HIP5 could be similar in terms of protein structure. It is tempting to further speculate that htt participates in nucleocytoplasmic transport.

### EXAMPLE 3

### Verification of PPIs

Comparison with literature-cited interactions revealed that more than 80% of the two-hybrid interactions identified here are novel. For all network bait and prey proteins only 24 PPIs have been reported previously using two-hybrid methods, coimmunoprecipitations or affinity chromatography-based techniques; 18 of these were confirmed in our Y2H assays (FIG. 2a, Table 2). Failure to detect interactions may result from the high stringency of our particular two-hybrid system. However, in most cases the occurrence of false negatives can be explained by the lack of essential domains in one of the protein fragments used. For example, an interaction between p53 and hADA3 has been described³², with the first 214 amino acids of hADA3 being essential for this interaction. It escaped our two-hybrid analysis, because a C-terminal hADA3 fragment (amino acids 235432) was used. For the same reason, an interaction between p53 and BARD1 or between KPNA2 and KPNB1 was not observed.

Beside false negatives, the two-hybrid assay is also prone to create false positive results⁹. Addressing this issue we performed a series of pull-down and overlay assays and thereby confirmed several of the two-hybrid PPIs independently. Proteins were expressed as GST-fusions in E coli and as HA-fusions in COS1 cells. After immobilization of the GST-fusion protein to beads or nitrocellulose membranes the respective partner was affinity-purified from a COS1 cell extract and binding was detected by immunoblotting. Using these assays, 22 physical interactions, central to the HD network, were verified (FIG. 2a). The results of some in vitro GST pull-down assays are shown in FIG. 3. For example HD510Q17 interacts with HIP1, GIT1, PIASy, FEZ1 and HIP11, and HIP5 binds to HD510Q68, GIT1, HBO1, PLIP and FEZ1 (FIG. 3). In total, 35 two-hybrid interactions were verified independently either in previous studies or by our in vitro binding assays (FIG. 2a).

### EXAMPLE 4

### GIT1 Promotes htt Aggregation In Vivo

The formation of insoluble polyQ-containing protein aggregates is a pathological hallmark of HD. Several lines of evidence link htt aggregation to disease progression and the development of motor symptoms. We screened network proteins for their potential to enhance htt aggregation in a cell-based aggregation assay¹⁴. In this assay, formation of SDS-insoluble htt aggregates in mammalian cells, that have been cotransfected with constructs encoding an N-terminal htt fragment with 68 glutamines (HD169Q68) and a network protein of interest, is monitored by filter retardation¹⁴ HD169Q68 per se has only a low propensity to form insoluble aggregates in HEK293 cells. However, as shown in FIG. 4a coexpression of the htt-interacting protein GIT1 strongly promotes the formation of HD169Q68 aggregates, whereas coexpression of PIASy, HIP5, HP28, PFN2, FEZ1 and BARD1 has no discernable effect. Thus, GIT1 is a potential modifier of HD pathogenesis, which may influence the rate of formation of insoluble htt aggregates in vivo.

Furthermore, probing of the insoluble HD169Q68 aggregates with an anti-GIT1 antibody revealed that GIT1 does not only stimulate aggregation but is also an integral part of the insoluble aggregates (FIG. 4a). This suggests that GIT1 promotes aggregation through direct binding to mutant htt.

The interaction between GIT1 and htt was confirmed by coimmunoprecipitation from COS1 cells transfected with constructs encoding HD510Q68 and HA-GIT1. Forty hours post transfection cell extracts were prepared and treated with antiserum against GIT1. HD510Q68 and HA-GIT1 were detected in the immunoprecipitate on Western blots with anti-hft antibody 4C8 and anti-HA antibody 12CA5, respectively (FIG. 4b).

The GIT1-htt interaction was also detected in human brain. Protein extracts prepared from human cortex were treated with the anti-htt antibodies CAG53b and HD1, and the precipitate was probed for the presence of GIT1 (FIG. 4c). Full length GIT1, migrating at about 90 kDa³³, was precipitated by both ant-htt antibodies in a concentration dependent manner, indicating the existence of a complex between htt and GIT1 in neurons.

Finally, we performed colocalisation studies of htt and GIT1 in COS1 cells using immunofluorescence microscopy. In cells expressing HD510Q68 or GIT1 alone a diffuse cytoplasmic staining was observed for each protein (FIG. 4d). However, when GIT1 and mutant htt were coexpressed, large perinuclear structures, most likely reflecting protein aggregates, appeared almost exclusively. These structures contained both GIT1 and htt. The images further substantiate the findings that GIT1 and htt bind to each other and that GIT1 is a potent enhancer of mutant htt aggregation.

### EXAMPLE 5

### GIT1 Localises to htt Aggregates in HD Transgenic Mouse and Patient Brains

The finding of colocalisation of htt and GIT1 within aggregates in transfected COS1 cells suggests that GIT1 might also be a component of htt aggregates in vivo. To investigate this possibility we first assessed the distribution of GIT1 in brains of R6/1 transgenic mice expressing a human htt exon 1 protein with 150 glutamines³⁴. In wildtype mice, GIT1 immunoreaction product was found diffuse in the cytoplasm and nuclei of neurons throughout the brain. In R6/1 brains, in addition to the diffuse staining, GIT1 immunoreactivity was also present in large nuclear and cytoplasmic puncta similar to htt aggregates (FIG. 5a). To further confirm these data, we examined the subcellular distribution of GIT1 in cortex from HD patient brains and healthy individuals (FIG. 5b). In patient brains, GIT1 antibodies labelled neuronal nuclear inclusions as well as neuropil aggregates characteristic of HD brains³⁵. In contrast, neurons from control brains only showed a diffuse nuclear and cytoplasmic GIT1 immunostaining. In fact, in colocalisation studies performed in HD brain sections, GIT1 positive aggregates were also labelled with anti-htt antibody 2B4, indicating that both proteins coaggregated in vivo (FIG. 5c). This observation raises the possibility that an alteration of the neuronal GIT1 subcellular distribution contributes to HD pathogenesis.

Part II: Verification and Further Results

### EXAMPLE 6

### Experimental Procedures

Antibodies

A polyclonal antibody (pAb) against GIT1 was generated by injection of purified His6-tagged GIT1 (aa 368-587) into a rabbit. The resulting GIT1 pAb (C-GIT1) was affinity purified using immobilized GIT1 protein. The pAb NT-GIT1 recognizes the first 100 aa of GIT1 (Santa Cruz Biotechnology), the monoclonal antibody (mAb) CT-GIT1 (Transduction Laboratories) is specific for the last 106 amino acids of GIT1. For all three Abs, no cross-reaction with GIT2 was observed (FIG. 13). The pAbs against GAPDH (Wanker et al., 1997) and htt [CAG53b (Davies et al., 1997) and HD1 (Scherzinger et al., 1997)] were described. Commercially available antibodies were anti-GST pAb (Amersham Pharmacia), anti-HA mAb 12CA5 (Roche Diagnostics), anti-htt pAb EM48 (Gutekunst et al., 1999), anti-htt mAb 2B4 (Lunkes et al., 2002), anti-htt mAb 4C8 (Chemicon) and anti-EEA1 pAb (Santa Cruz Biotechnology). As secondary antibodies for immunofluorescence microscopy, Cy3-(dianova) and Alexa 488-(MoBiTec) conjugated IgGs were used.

Strains and Plasmids

The yeast strains used for two-hybrid analysis were L40 ccua [MATa his3D200 trp1-901 leu2-3,112 LYS2::(lexAop)4-HIS3 ura3::(lexAop)8-lacZ ADE2::(lexAop)8-URA3 GAL4 gal80 can1 cyh2] and L40 cca [MATa his3D200 trp1-910 leu2-3,112 ade2 LYS2::(lexAop)4-HIS3 URA3::(lexAop)8-lacZ GAL4 gal80 can1 cyh2].

Plasmids pHD510Q17 and pHD510Q68 were generated by insertion of fragments coding for HD510Q17 and HD510Q68 into pcDNA-1 (Invitrogen). pHD169Q68 was derived from pHD510Q68 by deletion of the XhoI-XhoI fragment encoding aa 170-510 of human htt. pV5-HD169Q68 was generated by inserting the EcoRI-XhoI fragment from pHD510Q68 into pcDNA3.1/5-HIS (Invitrogen). Full-length GIT1 (aa 1-770) was amplified by PCR from the cDNA clone IMAGp958H111245Q2 (RZPD, Germany) using the primers GIT1-F/GIT1-R and subcloned into the EcoRI-BglII site of pTL-HA (HA-GIT1). The GIT2 cDNA (aa 1-759) was PCR amplified with the primers GIT2-F/GIT2-R and subcloned into the XhoI-NotI site of pTL-HA (HA-GIT2). The primer sequences were as follows: GIT1-F (5′-CGGMTTCATGTCCCGAAAGGGGCCGCG-3′), GIT1-R (5′-GGMGATCT GGTCACTGCTTCTTCTCTCGGG-3′), GIT2-F (5′-ACGCGTCGACCATGTCGAAA CGGCTCCG-3′) and GIT2-R (5′-ATAAGAATGCGGCCGCGCCCTGCCCTTGCTA GTTG-3′).

Library Screening

Plasmids encoding baits were transformed into L40 ccua, tested for the absence of reporter gene activity and cotransformed with a human fetal brain cDNA library (Clontech). For each transformation, 1×10⁶ independent transformants were plated onto minimal medium lacking tryptophan, leucine, histidine and uracil (SDIV medium) and incubated at 30° C. for 5 to 10 days. Clones were picked into microtitre plates and grown overnight in liquid minimal medium lacking tryptophan and leucine (SDII medium). Then, they were spotted onto nylon membranes placed on SDIV agar plates. After incubation for 4 days, the membranes were subjected to a b-galactosidase (b-GAL) assay. Plasmids were prepared from positive clones and characterized by sequencing. For retransformation assays, plasmids encoding baits and preys were cotransformed into L40 ccua and plated onto SDIV medium.

Array Mating Screen

Plasmids encoding baits and preys were transformed into strains L40 ccua and L40 cca, respectively. L40 cca clones were arrayed in 96-well microtitre plates and mixed with a single L40 ccua clone for interaction mating. Diploid cells were transferred onto YPD medium plates and, after incubation for 24 h at 30° C., onto SDII medium plates for additional 72 h at 30° C. For two-hybrid selection, diploid cells were transferred onto SDIV medium plates with and without nylon membranes and incubated for 5 days at 30° C. The nylon membranes were subjected to the b-GAL assay. Positive clones were verified by cotransformation assays.

Protein Expression and Verification Assays

For verification experiments, cDNA fragments encoding baits and preys were subcloned into pGEX derivatives (Stratagene) or pTL-HA (Sittler et al., 1998). GST-fusion proteins were expressed in E. coli BL21-codon Plus™ RP (Stratagene) and affinity purified on glutathione agarose beads (Sigma) (Wanker et al., 1997). COS-1 cells were transfected with mammalian expression plasmids and lysed as described (Sittler et al., 1998). For in vitro binding assays, 30 μg of GST or GST fusion protein were immobilized on glutathione agarose beads and incubated with 500 μg COS-1 cell extract containing HA-tagged fusion protein for 2 h at 4° C., in binding buffer [50 mM HEPES-KOH pH 7.4, 150 mM NaCl, 10% glycerol, 1% NP-40, 1 mM EDTA, 20 mM NaF, 1 mM DTT, 0.1% Triton X-100, protease inhibitors (Roche Diagnostics)]. After centrifugation and extensive washing, bound proteins were eluted and analyzed by SDS-PAGE and Western blotting.

Coimmunoprecipitation experiments were performed as previously described (Sittler et al., 1998). For immunofluorescence microscopy, COS-1 cells were grown on cover slips and cotransfected with plasmids encoding N-terminal htt V5-HD169Q68 and/or C-terminal HA-GIT1-CT. 40 h post-transfection, cells were treated with 2% paraformaldehyde. Immunolabeling was performed with anti-C-GIT1 (1:500) and with anti-V5 (1:300) Abs. Nuclei were counterstained with Hoechst. For subcellular localization of endogenous GIT1 and htt, differentiated PC12 and SH-SY5Y cells were used. PC12 cells were treated with 50 ng/ml NGF and grown on cover slips for 6 d. SH-SY5Y cells were serum starved for 24 h and then differentiated with 10 nM IGF-I for 30 min. Cells were labeled with C-GIT1 (1:20) and 4C8 (1:20) Abs and viewed with a confocal microscope LSM510 (Zeiss).

Filter Retardation Assay

HEK293 cells coexpressing HD169Q68 and selected network proteins were harvested 48 h post-transfection. Cell lysates were boiled in 2% SDS, 50 mM DTT for 5 min. Aliquots containing 12.5, 25 or 50 μg of total protein were used for filtration on cellulose acetate membranes (Scherzinger et al., 1997). SDS-resistant aggregates were detected using anti-CAG53b or anti C-GIT1 pAbs.

Inhibition of GIT1 Expression by siRNA

For silencing of endogenous GIT1 expression, HEK293 cells were transfected with the siRNA duplex siRNA-GIT1 (5′-AAGCCTGGATGGAGACCTA GA-3′) using TransMessenger (Qiagen) or Lipofectamin 2000 (Invitrogen) transfection reagents. 48 h post transfection, cell lysates were analyzed for GIT1 expression by immunoblotting using C-GIT1 Ab. To examine the effect of endogenous GIT1 silencing on htt aggregation, HEK293 cells were cotransfected with pHD169Q68 and siRNA-GIT1 and subjected to filtration after 72 h.

Detection of GIT1 in R6/1 Mouse and Human HD Brains

For immunocytochemistry, mice were deeply anaesthetized and perfused through the left cardiac ventricle with 4% paraformaldehyde in 0.1 M phosphate buffer. Brains were removed and postfixed overnight in 4% paraformaldehyde. Sections were processed for immunocytochemistry as described (Gutekunst et al., 1999). EM48 (1:1000) and C-GIT1 (1:100) pAbs were used.

Tissues from 8 human HD and 7 control brains were used in this study. Two HD cases were classified as grade 3 of neuropathological severity, six cases as grade 4. Standard protocols were used (Lunkes et al., 2002) for immunolabeling with 2B4 mAb (1:200) and C-GIT1 pAb (1:50). For Western analysis of total protein lysates from frontal cortex, the C-GIT1 pAb (1:300) was used.

### EXAMPLE 7

### Two-Hybrid Screens

To generate a PPI network for HD, we used a combination of library and matrix yeast two-hybrid screens (FIG. 7A). Previous studies have shown that htt potentially participates in clathrin-mediated endocytosis, apoptosis, vesicle transport, cell signaling, morphogenesis and transcriptional regulation (Harjes and Wanker, 2003; Li and Li, 2004). For this reason, we selected 50 cDNAs encoding proteins involved in these processes, including 5 different N-terminal htt fragments, as well as proteins known to interact with htt, for subcloning into a DNA binding domain vector to express LexA fusion proteins as baits (Suppl. Table 1). The resulting plasmids were sequenced and introduced into yeast strain L40 ccua, which carries three reporter genes, HIS3, URA3 and lacZ, for two-hybrid interaction analysis.

Forty of these baits did not activate the reporters by themselves and were used individually for cotransformation screening of a human fetal brain cDNA library expressing GAL4 activation domain (AD) hybrids as preys. In each screen, 1×10⁶ auxotrophic transformants were tested on selective plates, and 1-50 positive colonies were typically obtained. Restriction analysis and sequencing revealed that about 12% of all positive clones expressed preys with correct in-frame sequences, while 88% of the clones contained plasmids with out-of-frame sequences or sequences from non-protein-encoding regions, which were discarded. 27 preys were identified only once, while the other 11 were found up to four times as independent AD fusions. Plasmids with the longest coding regions were used for subsequent studies. The preys identified by the library two-hybrid screens were tested together with their respective baits for activation of reporter gene expression in cotransformation assays. Only prey/bait combinations that activated the reporter gene expression in two independent cotransformation assays were selected for further two-hybrid studies and in vitro pull-down assays (FIG. 9). Starting with 40 baits in the library and subsequent cotransformation screens, we identified 41 PPIs among 18 bait and 38 prey proteins.

For a second round of two-hybrid screens, cDNAs encoding 12 prey proteins were selected from literature verified interactions and from interactions confirmed by in vitro binding experiments (Suppl. Table 2), and subcloned into a DNA binding domain vector. The resulting baits were tested for autoactivation, and 10 were screened against a human fetal brain cDNA library as described above. We identified another 14 PPIs among 5 bait and 13 prey proteins. Nine preys were found once and 4 were discovered multiple times as independent AD fusions. All interactions were confirmed by cotransformation assays.

Finally, an array-mating screen was performed to connect bait and prey proteins identified in the cDNA library transformation screens (FIG. 7A). L40 cca yeast cultures were transformed with plasmids encoding the 51 prey proteins obtained in the first and second round of cDNA library screens and arrayed in 96-well microtiter plates. Prey cDNAs were also subcloned into DNA binding domain vectors and introduced into an L40ccua strain to generate additional baits for interaction mating. Including the ones already used for the library screens, we arrived at 46 baits, which did not autoactivate the reporter genes (Table 7). These baits were used individually for mating against the matrix of prey proteins. Diploid yeast clones, formed on YPD plates, were selected on agar SDII plates, and further transferred by a spotting robot onto SDIV plates to select for Y2H interactions (FIG. 7B). We examined 2346 (51×46). pair wise combinations of baits and preys in the mating assay reproducing all 55 two-hybrid interactions, which had been found in the library screens. In addition, 131 new PPIs were found by interaction mating and subsequently reproduced in cotransformation assays. Using this combination of library and matrix two-hybrid screens, a total of 186 PPIs among. 35 bait and 51 prey proteins could be identified (FIG. 8A);

Sequence analysis of the cDNAs revealed ORFs ranging from 82 to 728 amino acids in size (Table 7). In a systematic Blast search, 77 of the 86 bait and prey protein fragments were identical to a SwissProt or TrEMBL protein entry (http://us.expasy.org/sprott/). Nine proteins showed 75-99% identity to their best respective database hit and either contained single amino acid substitutions, variable polyQ lengths or small regions of sequence variation. Uncharacterized proteins were named according to their interaction partners.

This chapter describes the whole yeast two hybrid screening procedure and obtained fundamental data. A full description of our final datasets are shown in tables 6 to 9. Table 6 contains a compilation of all found protein-protein interactions in the Huntington's disease protein network. Some of these interactions are already known and literature-cited. A dataset which describes only new identified interactions will be found in Table 9. Table 7 characterizes all proteins involved in the protein network. Most of these proteins are known from different databases but some proteins are still unknown (Table 8). Nucleic acid and amino acid sequence data for all network-proteins are available from FIG. 6.

### EXAMPLE 8

### Functional Assignment of Yeast Two-Hybrid Data

To chart two-hybrid interactions identified in this study, previously reported, or verified by independent methods, a matrix representation was used (FIG. 8A). We assigned a subcellular localization to each network protein by examining various sources of literature and, based on the experimental data, we grouped the proteins into six broad functional categories (FIG. 8A, Table 7). 18 proteins in the HD network are involved in transcriptional regulation or DNA maintenance; 14 proteins mainly participate in cytoskeletal and transport processes. We assigned 7 proteins to cellular signaling and fate, another 5 to protein synthesis and turnover, and 3 proteins to cellular metabolism. 16 proteins of unknown function were identified, participating in 72 interactions. The number of interactions identified for each protein varied from 1 to 24, with 2.6 interaction partners on average. Interestingly, proteins such as htt, BARD1, GADD45G, HIP5, HZFH, PIASy, BAIP3 or VIM interact with more than 15 other proteins, forming hubs in the HD network, while 15 proteins have only one interaction partner.

For htt, 19 different interacting partners from various functional groups were identified, of which HIP1, CA150, SH3GL3 and HYPA had been described previously (Harjes and Wanker, 2003). 6 of the htt partners are involved in transcriptional regulation and DNA maintenance, 4 function in cellular organization and transport and 3 in cellular signaling, supporting the hypothesis that htt is involved in these processes. Moreover, we have detected 6 novel htt interacting proteins of unknown function termed HIP5, HIP11, HIP13, HIP15, HIP16, and CGI-125.

Using 5 different N-terminal htt fragments as baits, the potential htt-binding sites of 13 interaction partners were mapped (FIG. 8A). For the proteins CA150, HYPA, PNF2, SH3GL3, CGI-125 and HIP13, however, a conclusive determination of the htt binding region was not possible with the two-hybrid assay, because these proteins bound to HDexQ20, HDexQ51 and HD1.7, but not to HDd1.0 (FIG. 8A). We suggest that these proteins bind to the htt exon 1 fragment, but this binding region might be masked in the HDd1.0 protein, while it is accessible in the HD1.7 fragment. Interestingly, we found that HP28 and HIP15 bind to HDexQ51, but not to HDexQ20, HD1.7 and HD1.0, indicating that the interaction of these proteins with htt is enhanced by the expanded polyQ repeat. Thus, HP28 and HIP15 may be disease specific htt interactors.

To generate a more comprehensive HD interaction map, we supplemented bur two-hybrid network (red diamonds) with all 38 known direct htt interaction partners (Suppl. Table 4 and FIG. 8B, blue squares). Furthermore, we added 83 human proteins (green triangles), identified from protein interaction databases HPRD, MINT, and BIND that bridge any two proteins in our extended network. Using this approach, we obtained an interaction network for htt containing a total of 181 proteins and 591 PPIs (FIG. 8B and Suppl. Table 5).

### EXAMPLE 9

### Verification of PPIs

Comparison with literature-cited interactions revealed that more than 89% of the two-hybrid interactions identified are unknown. 30 PPIs have been reported previously using two-hybrid methods, coimmunoprecipitations or affinity chromatography-based techniques; 21 of these were detected in our Y2H assays (FIG. 8A, Suppl. Table 3). In most cases, the occurrence of false negatives can be explained by the lack of essential domains in one of the protein fragments. For example, an interaction between p53 and hADA3 has been described (Wang et al., 2001), with the first 214 amino acids of hADA3 being essential for this interaction. It escaped our two-hybrid analysis, because a C-terminal hADA3 fragment (amino acids 235-432) was used.

Failure to detect interactions may also result from the high stringency of our two-hybrid assay, which can be attributed to low protein expression levels and the simultaneous use of three reporters. Our system is particularly designed to minimize false positives, which are known to occur frequently in two-hybrid assays (von Mering et al., 2002). To determine the rate of false positives in our system, we directly assessed 54 interactions from the two-hybrid network by in vitro pull-down experiments, mainly focusing on htt and its immediate interaction partners. Proteins were expressed as GST-fusions in E. coli, and their interacting partners as HA-fusions in COS-1 cells. After immobilization of GST-fusion proteins to beads, the potential interaction partners were pulled down from COS-1 cell extracts. Binding was detected by SDS-PAGE and immunoblotting. Using this assay, 35 interactions representing 32 different protein pairs were verified successfully (FIG. 9). Failure to detect an interaction by GST pull-down assays could be due to low protein expression levels or the lack of appropriate protein modifications. Therefore, the 19 non-verified protein-protein interactions are still valid until further experiments show contradictory results. The rate of 64.8% verified interactions suggests that in our Y2H network false positives might appear less frequently than described for other PPI studies (von Mering et al., 2002).

### EXAMPLE 10

### GIT1 Promotes htt Aggregation

Several lines of evidence indicate that aggregation of mutant htt is linked to disease progression and the development of motor symptoms (Davies et al., 1997; Sanchez et al., 2003). Therefore, cellular proteins that influence aggregate formation are potential modulators of disease pathogenesis. In order to identify such proteins, we screened all 19 direct htt interaction partners (FIG. 8A) for their ability to enhance htt aggregation in a cell-based assay (Sittler et al., 1998). In this assay, HEK293 cells were cotransfected with constructs encoding an aggregation prone N-terminal htt fragment with 68 glutamines (HD169Q68) and a network protein. After 48 h, formation of SDS-insoluble htt aggregates was analyzed by a filter retardation assay (Scherzinger et al., 1997). In this time period HD169Q68 by itself formed only few aggregates. In comparison, coexpression of the C-terminal GIT1 fragment found in the Y2H screens (GIT1-CT) increased the amount of htt aggregates 3-fold (FIG. 10A). Coexpression of HD169Q68 with other htt-interacting proteins, on the other hand, did not enhance htt aggregation (data not shown).

It has been described previously that GIT1 and its homologue p95-APP1 are able to form homo- and heterodimers in vitro and in vivo (Kim et al., 2003; Paris et al., 2003). Therefore, we wondered whether GIT1-CT by itself is able to form SDS-insoluble protein aggregates in mammalian cells. As shown in FIG. 10A, we did not detect aggregates in the filter retardation assay upon transient overexpression of GIT1-CT. However, in cells coexpressing HD169Q68 and GIT1-CT, stable SDS-resistant aggregates immunoreactive with the anti-C-GIT1 antibody were formed, indicating that both proteins coaggregate in cells, and that GIT1-CT is an integral part of the insoluble htt aggregates (FIG. 10A).

Next, we tested whether full-length GIT1 is able to accelerate htt aggregation in mammalian cells. Analysis by filter retardation assay revealed that full-length GIT1 enhances htt aggregation in a dose dependent manner (FIG. 10B). However, compared to GIT1-CT, it was less efficient in stimulating HD169Q68 aggregation in the cell model, indicating that the N-terminally truncated GIT1 fragment is a more potent enhancer of htt aggregation than the full-length protein.

As previous studies have shown that the expression of C-terminal GIT1/p95-APP1 fragments induces the formation of large vesicular structures in mammalian cells (Di Cesare et al., 2000; Matafora et al., 2001), we analyzed the effect of GIT1-CT on HD169Q68 aggregation by indirect immunofluorescence microscopy. We found that expression of GIT1-CT alone induced the accumulation of large vesicular structures in the perinuclear region (FIG. 10Cb). In comparison, when HD169Q68 was expressed alone, the protein was distributed in the cytoplasm, and no large aggregates or inclusion bodies were observed (FIG. 10Ca). However, when HD169Q68 and GIT1-CT were coexpressed (FIG. 10Cd-f), htt was almost exclusively detected in the perinuclear vesicles (FIG. 10Cd), indicating that GIT1-CT overexpression induces the relocalization of htt to membranous structures. A similar effect was observed when full-length GIT1 and HD169Q68 were coexpressed in COS1 cells, however, the rate of vesicle formation and htt recruitment was lower, compared to GIT1-CT/HD169Q68 expressing cells (data not shown). The colocalization of GIT1 with the early endosomal marker EEA1 is shown in FIG. 10Cc. Together, these results suggest that the enhancement of HD169Q68 aggregation in mammalian cells is due to the recruitment of mutant htt into vesicular structures induced by overexpression of GIT1 or GIT1-CT.

### EXAMPLE 11

### GIT1 is Crucial for the Formation of htt Aggregates in Mammalian Cells

Next, we investigated whether endogenous GIT1 promotes htt aggregation in mammalian cells. In order to reduce endogenous GIT1 levels in HEK293 cells, we employed the short-interfering RNA (siRNA) technology (Elbashir et al., 2001). Cells were cotransfected with HD169Q68 and GIT1-specific siRNA, and silencing of endogenous GIT1 was monitored 48 h post transfection by Western blot analysis (FIG. 10D). We found that siRNA treatment specifically reduced endogenous GIT1 by ˜80% and caused a strong decrease of HD169Q68 aggregate formation (FIG. 10E). After incubation for 72 h, SDS-resistant HD169Q68 aggregates were detected in untreated, but not in siRNA treated cells. This indicates that physiological levels of GIT1 are critical for htt aggregation in mammalian cells, and that an inhibition of GIT1 expression dramatically slows down aggregate formation. A similar effect was also obtained when GIT1-specific siRNA was applied to cells overexpressing GIT1-CT and HD169Q68 proteins (data not shown).

### EXAMPLE 12

### Verification of the htt-GIT1 Interaction

The interaction between GIT1-CT and htt was confirmed by coimmunoprecipitation from COS-1 cells transfected with constructs encoding the first 510 amino acids of htt with 68 glutamines (HD510Q68) and an N-terminally truncated hemagglutinin (HA) tagged HA-GIT1-CT (aa 249-770) protein. 40 h post-transfection, cell extracts were prepared and treated with GIT1 antiserum. HD510Q68 and HA-GIT1-CT were detected in the immunoprecipitates on Western blots with anti-htt antibody 4C8 and anti-HA antibody 12CA5, respectively (FIG. 11A).

The GIT1-htt interaction was also detected in healthy human brain. Protein extracts prepared from cortex were treated with the anti-htt antibodies CAG53b and HD1, and the precipitate was probed for the presence of GIT1 (FIG. 11B) with a GIT1 specific antibody (NT-GIT1; FIG. 13). Full length GIT1, migrating at about 95 kDa (Vitale et al., 2000), was precipitated by both anti-htt antibodies in a concentration dependent manner, indicating that a protein complex containing htt and GIT1 is formed under physiological conditions.

Next, we examined the colocalization of endogenous htt and GIT1 in differentiated PC12 cells by confocal immunofluorescence microscopy. Both proteins were mainly detected in the cytoplasm, but were also present in the neurite-like extensions (FIG. 11Cab). Colocalization, indicated in yellow, was visible in cytoplasmic complexes in the perinuclear region (FIG. 11Cc) as well as in a number of intracellular structures scattered throughout the neuritic extensions. GIT1 was also detected in adhesion-like structures at the tip of the extensions, as previously reported (Di Cesare et al., 2000; Manabe Ri et al., 2002). These regions, however, did not contain htt protein. Similar results were obtained when the endogenous localization of GIT1 and htt was analyzed in differentiated neuroblastoma SH-SY5Y cells using confocal immunofluorescence microscopy (FIG. 11Cd-f).

### EXAMPLE 13

### GIT1 Localizes to htt Aggregates in Patient Brain

Our findings suggest that GIT1 might also be a component of neuronal inclusions containing htt aggregates in brain of HD patients and transgenic animals (Davies et al., 1997; DiFiglia et al., 1997). To investigate this possibility, we first assessed the distribution of GIT1 in brain slices of R6/1 transgenic mice expressing a human hft exon 1 protein with 150 glutamines (Mangiarini et al., 1996). In wild type mice, GIT1 specific immunoreactivity was diffused in the cytoplasm and nuclei of neurons throughout the brain. In R6/1 brain, however, in addition to a diffuse staining, GIT1 immunoreactivity was also present in large nuclear and cytoplasmic puncta containing htt aggregates (FIG. 12A). To further confirm these data, we examined the subcellular distribution of GIT1 in HD patient and healthy cortex (FIG. 12B). In patient brain, GIT1 specific antibodies labeled neuronal nuclear inclusions as well as the neuropil aggregates characteristic of HD (DiFiglia et al., 1997). In contrast, neurons from control tissue showed only diffuse nuclear and cytoplasmic GIT1 immunostaining. FIG. 12C shows colocalization of htt and GIT1 in neuronal nuclear inclusions.

### EXAMPLE 14

### GIT1 is Degraded in HD Patient Brain

The presence of GIT1 in protein extracts from HD affected and unaffected cortex was also analyzed by SDS-PAGE and immunoblotting. As shown in FIG. 12D, full-length GIT1 protein migrating at about 95 kDa was detected in healthy brain (FIG. 12D), but was significantly reduced in HD. Interestingly, in HD, but not in control brain, prominent GIT1 degradation products migrating at about 25-50 kDa were detected with the C-terminal GIT1 antibody C-GIT1 (FIG. 12D). In strong contrast, no such products were observed when the N-terminal GIT1 antibody NT-GIT1 directed against the ARF-GAP domain was used (data not shown). This indicates the formation of large amounts of N-terminally truncated GIT1 fragments in HD brain, which may be a significant factor in disease pathogenesis.

### ADDITIONAL REFERENCES

1. Tucker, C. L., Gera, J. F. & Uetz, P. Towards an understanding of complex protein networks. Trends Cell. Biol. 11, 102-106 (2001).
2. Alberts, B. The cell as a collection of protein machines: preparing the next generation of molecular biologists. Cell 92, 291-294 (1998).
3. Legrain, P., Wojcik, J. & Gauthier, J. M. Protein—protein interaction maps: a lead towards cellular functions. Trends Genet 17, 346-352 (2001).
4. Fields, S. & Song, O. A novel genetic system to detect protein-protein interactions. Nature 340, 245-246 (1989).
5. Uetz, P. et al. A comprehensive analysis of protein-protein interactions in Saccharomyces cerevisiae. Nature 403, 623-627 (2000).
6. Ito, T. et al. A comprehensive two-hybrid analysis to explore the yeast protein interactome. Proc. Natl Acad. Sci. USA 98, 4569-74 (2001).
7. Rain, J. C. et al. The protein-protein interaction map of Helicobacter pylori. Nature 409, 211-5 (2001).
8. Bader, G. D. & Hogue, C. W. Analyzing yeast protein-protein interaction data obtained from different sources. Nature Biotechnol. 20, 991-997 (2002).
9. von Mering, C. et al. Comparative assessment of large-scale data sets of protein-protein interactions. Nature 417, 399-403 (2002).
10. Walhout, A. J. et al. Integrating Interactome, Phenome, and Transcriptome Mapping Data for the C. elegans Germline. Curr. Biol. 12, 1952-1958 (2002).
11. Walhout, A. et al. Protein interaction mapping in C. elegans using proteins involved in vulval development. Science 287, 116-122 (2000).
12. Fromont-Racine, M., Rain, J. & Legrain, P. Toward a functional analysis of the yeast genome through exhaustive two-hybrid screens. Nature Genet. 16, 277-282 (1997).
13. Davies, S. W. et al. Formation of neuronal intranuclear inclusions underlies the neurological dysfunction in mice transgenic for the HD mutation. Cell 90, 537-548 (1997).
14. Scherzinger, E. et al. Huntingtin-encoded polyglutamine expansions form amyloid-like protein aggregates in vitro and in vivo. Cell 90, 549-558 (1997).
15. Cattaneo, E. et al. Loss of normal huntingtin function: new developments in Huntington's disease research. Trends Neurosci. 24, 182-188 (2001).
16. Li, X. et al. A huntingtin-associated protein enriched in brain with implications for pathology. Nature 378, 398-402 (1995).
17. Wanker, E. et al. HIP-I: a huntingtin interacting protein isolated by the yeast two-hybrid system. Hum. Mol. Genet. 3, 487-495 (1997).
18. Sittler, A. et al. SH3GL3 associates with the Huntingtin exon 1 protein and promotes the formation of polygin-containing protein aggregates. Mol. Cell 4, 427-436 (1998).
19. Milo, R. et al. Network motifs: simple building blocks of complex networks. Science 298, 824-7 (2002).
20. Schwikowski, B., Uetz, P. & Fields, S. A network of protein-protein interactions in yeast. Nature Biotechnol. 17, 1030-1032 (2000).
21. Cha, J. H. Transcriptional dysregulation in Huntington's disease. Trends Neurosci. 23, 387-392 (2000).
22. Holbert, S. et al. The Gin-Ala repeat transcriptional activator CAI 50 interacts with huntingtin: neuropathologic and genetic evidence for a role in Huntington's disease pathogenesis. Proc. Natl Acad. Sci. USA 98, 1811-1816 (2001).
23. Faber, P. et al. Huntingtin interacts with a family of WW domain proteins. Hum. Mol. Genet. 7, 1463-1474 (1998).
24. Hawkes, N. A. et al. Purification and characterization of the human elongator complex. J. Biol. Chem. 277, 3047-3052 (2002).
25. Liu, B., Gross, M., ten Hoeve, J. & Shuai, K. A transcriptional corepressor of Stat1 with an essential LXXLL signature motif. Proc. Natl Acad. Sci. USA 98, 3203-3207 (2001).
26. Sachdev, S. et al. PIASy, a nuclear matrix-associated SUMO E3 ligase, represses LEFL activity by sequestration into nuclear bodies. Genes Dev. 15, 3088-3103 (2001).
27. Engelender, S. et al. Huntingtin-associated protein 1 (HAPI) interacts with the p150^{Glued} subunit of dynactin. Hum. Mol. Genet 6, 2205-2212 (1997).
28. Waelter, S. et al. The huntingtin interacting protein HIP1 is a clathrin and alpha-adaptin-binding protein involved in receptor-mediated endocytosis. Hum. Mol. Genet. 10, 1807-1817 (2001).
29. Witke, W. et al. In mouse brain profilin I and profilin 11 associate with regulators of the endocytic pathway and actin assembly. EMBO J. 17, 967-976 (1998).
30. Chook, Y. & Blobel, G. Karyopherins and nuclear import. Curr. Opin. Struct. Biol. 11, 703-715 (2001).
31. Andrade, M. A., Petosa, C., O'Donoghue, S. I., Muller, C. W. & Bork, P. Comparison of ARM and HEAT protein repeats. J. Mol. Biol. 309, 1-18 (2001).
32. Wang, T. et al. hADA3 is required for p53 activity. EMBO J. 20, 6404-6413 (2001).
33. Vitale, N. et al. GIT proteins, A novel family of phosphatidylinositol 3,4,5-trisphosphate-stimulated GTPase-activating proteins for ARF6. J. Biol. Chem. 275, 13901-13916 (2000).
34. Mangiarini, L. et al. Exon 1 of the Huntington's disease gene containing a highly expanded CAG repeat is sufficient to cause a progressive neurological phenotype in transgenic mice. Cell 87, 493-506 (1996).
35. DiFiglia, M. et al. Aggregation of huntingtin in neuronal intranuclear inclusions and dystrophic neurites in brain. Science 277, 1990-1993 (1997).
36. Aebersold, R. & Mann, M. Mass spectrometry-based proteomics. Nature 422, 198-207 (2003).
37. Bernstein, C., Bernstein, H., Payne, C. M. & Garewal, H. DNA repair/pro-apoptotic dual-role proteins in five major DNA repair pathways: fail-safe protection against carcinogenesis. Mutat. Res. 511, 145-178 (2002).
38. Becher, M. W. et al. Intranuclear neuronal inclusions in Huntington's disease and dentatorubral and pallidoluysian atrophy: correlation between the density of inclusions and IT15 CAG triplet repeat length. Neurobiol. Dis. 4, 387-397 (1998).
39. Hernandez-Deviez, D. J., Casanova, J. E. & Wilson, J. M; Regulation of dendritic development by the ARF exchange factor ARNO. Nature Neurosci. 5, 623-624 (2002).
40. Di Cesare, A. et al. p95-APP1 links membrane transport to Rac-mediated reorganization of actin. Nature Cell Biol. 8, 521-530 (2000).
41. Zhang, H., Webb, D. J., Asmussen, H. & Horwitz, A. F. Synapse formation is regulated by the signaling adaptor GIT1. J. Cell. Biol. 161, 131-142 (2003).
42. Graveland, G. A., Williams, R. S. & DiFiglia, M. Evidence for degenerative and regenerative changes in neostriatal spiny neurons in Huntington's disease. Science 227, 770-773 (1985).
43. Ko, J. et al. Interaction between liprin-alpha and GIT1 is required for AMPA receptor targeting. J. Neurosci. 23, 1667-1677 (2003).
44. Cha, J. H. et al. Altered brain neurotransmitter receptors in transgenic mice expressing a portion of an abnormal human Huntington disease gene. Proc. Natl Acad. Sci. USA 95, 6480-6485 (1998).
45. Kim, S. et al. The GIT family of proteins forms multimers and associates with the presynaptic cytomatrix protein Piccolo. J. Biol. Chem. 278, 6291-6300 (2003).
46. Scherzinger, E. et al. Self-assembly of polyglutamine-containing huntingtin fragments into amyloid-like fibrils: implications for Huntington's disease pathology. Proc. Natl Acad. Sci. USA 96, 4604-4609 (1999).
47. Gutekunst, C. A. et al. Nuclear and neuropil aggregates in Huntington's disease: relationship to neuropathology. J. Neurosci. 19, 2522-2534 (1999).
48. Lunkes, A. et al. Proteases acting on mutant huntingtin generate cleaved products that differentially build up cytoplasmic and nuclear inclusions. Mol. Cell 10, 259-269 (2002).
49. Davy A., Bello P., Thierry-Mieg N., Vaglio P., Hitti J., Doucette-Stamm L., Thierry-Mieg D., Reboul J., Boulton S., Walhout A J., Coux O., Vidal M. A protein-protein interaction map of the Caenorhabditis elegans 26S proteasome. EMBO Rep. 2001, September; 2(9): 821-8.
   50. Albertinazzi, C., Za, L., Paris, S., and De Curtis, I. (2003). ADP-Ribosylation Factor 6 and a Functional PIX/p95-APP1 Complex Are Required for Rac1B-mediated Neurite Outgrowth. Mol Biol Cell 14, 1295-1307.
51. Alberts, B. (1998). The cell as a collection of protein machines: preparing the next generation of molecular biologists. Cell 92, 291-294.
52. Bader, G. D., and Hogue, C. W. (2002). Analyzing yeast protein-protein interaction data obtained from different sources. Nature Biotechnol 20, 991-997.
53. Becher, M. W., Kotzuk, J. A., Sharp, A. H., Davies, S. W., Bates, G. P., Price, D. L., and Ross, C. A. (1998). Intranuclear neuronal inclusions in Huntington's disease and dentatorubral and pallidoluysian atrophy: correlation between the density of inclusions and IT15 CAG triplet repeat length. Neurobiol Dis 4, 387-397.
54. Cattaneo, E., Rigamonti, D., Goffredo, D., Zuccato, C., Squitieri, F., and Sipione, S. (2001). Loss of normal huntingtin function: new developments in Huntington's disease research. Trends Neurosci 24, 182-188.
55. Claing, A., Perry, S. J., Achiriloaie, M., Walker, J. K., Albanesi, J. P., Lefkowitz, R. J., and Premont, R. T. (2000). Multiple endocytic pathways of G protein-coupled receptors delineated by GIT1 sensitivity. Proc Natl Acad Sci USA97, 1119-1124.
56. Davies, S. W., Turmaine, M., Cozens, B. A., DiFiglia, M., Sharp, A. H., Ross, C. A., Scherzinger, E., Wanker, E. E., Mangiarini, L., and Bates, G. P. (1997). Formation of neuronal intranuclear inclusions underlies the neurological dysfunction in mice transgenic for the HD mutation. Cell 90, 537-548.
57. de Curtis, I. (2001). Cell migration: GAPs between membrane traffic and the cytoskeleton. EMBO Rep 2, 277-281.
58. Di Cesare, A., Paris, S., Albertinazzi, C., Dariozi, S., Andersen, J., Mann, M., Longhi, R., and de Curtis, I. (2000). p95-APP1 links membrane transport to Rac-mediated reorganization of actin. Nature Cell Biol 8, 521-530.
59. DiFiglia, M., Sapp, E., Chase, K. O., Davies, S. W., Bates, G. P., Vonsattel, J. P., and Aronin, N. (1997). Aggregation of huntingtin in neuronal intranuclear inclusions and dystrophic neurites in brain. Science 277, 1990-1993.
60. Elbashir, S. M., Harborth, J., Lendeckel, W., Yalcin, A., Weber, K., and Tuschl, T. (2001). Duplexes of 21-nucleotide RNAs mediate RNA interference in cultured mammalian cells. Nature 411, 494-498.
61. Ge, H., Walhout, A. J., and Vidal, M. (2003). Integrating ‘omic’ information: a bridge between genomics and systems biology. Trends Genet 19, 551-560.
62. Graveland, G. A., Williams, R. S., and DiFiglia, M. (1985). Evidence for degenerative and regenerative changes in neostriatal spiny neurons in Huntington's disease. Science 227, 770-773.
63. Gutekunst, C. A., Li, S. H., Yi, H., Mulroy, J. S., Kuemmerle, S., Jones, R., Rye, D., Ferrante, R. J., Hersch, S. M., and Li, X. J. (1999). Nuclear and neuropil aggregates in Huntington's disease: relationship to neuropathology. J Neurosci 19, 2522-2534.
64. Harjes, P., and Wanker, E. E. (2003). The hunt for huntingtin function: interaction partners tell many different stories. Trends Biochem Sci 28, 425-433.
65. Harper, P. S. (1996). Huntington's disease (London, W.B. Saunders Ltd.).
66. Jimenez-Sanchez, G., Childs, B., and Valle, D. (2001). Human disease genes. Nature 409, 853-855.
67. Kim, S., Ko, J., Shin, H., Lee, J., Lim, C., Han, J., Altrock, W., Garner, C., Gundelfinger, E., Premont, R., et al. (2003). The GIT family of proteins forms multimers and associates with the presynaptic cytomatrix protein Piccolo. J Biol Chem 278, 6291-6300.
68. Li, S.-H., and Li, X.-J. (2004). Huntingtin-protein interactions and the pathogenesis of Huntington's disease. Trends Genet 20, 146-154.
69. Lunkes, A., Lindenberg, K. S., Ben-Haiem, L., Weber, C., Devys, D., Landwehrmeyer, G. B., Mandel, J. L., and Trottier, Y. (2002). Proteases acting on mutant huntingtin generate cleaved products that differentially build up cytoplasmic and nuclear inclusions. Mol Cell 10, 259-269.
70. Manabe R1, R., Kovalenko, M., Webb, D. J., and Horwitz, A. R. (2002). GIT1 functions in a motile, multi-molecular signaling complex that regulates protrusive activity and cell migration. J Cell Sci 115, 1497-1510.
71. Mangiarini, L., Sathasivam, K., Seller, M., Cozens, B., Harper, A., Hetherington, C., Lawton, M., Troltier, Y., Lehrach, H., Davies, S. W., and Bates, G. P. (1996). Exon 1 of the Huntington's disease gene containing a highly expanded CAG repeat is sufficient to cause a progressive neurological phenotype in transgenic mice. Cell 87, 493-506.
72. Matafora, V., Paris, S., Dariozzi, S., and de Curtis, I. (2001). Molecular mechanisms regulating the subcellular localization of p95-APP1 between the endosomal recycling compartment and sites of actin organization at the cell surface. J Cell Sci 4, 4509-4520.
73. Milo, R., Shen-Orr, S., ltzkovitz, S., Kashtan, N., Chklovskii, D., and Alon, U. (2002). Network motifs: simple building blocks of complex networks. Science 298, 824-827.
74. Paris, S., Longhi, R., Santambrogio, P., and de Curtis, I. (2003). Leucine-zipper-mediated homo- and hetero-dimerization of GIT family p95-ARF GTPase-activating protein, PIX-, paxillin-interacting proteins 1 and 2. Biochem J 372, 391-398.
75. Perutz, M. F. (1999). Glutamine repeats and neurodegenerative diseases: molecular aspects. Trends Biochem Sci 24, 58-63.
76. Sanchez, I., Mahlke, C., and Yuan, J. (2003). Pivotal role of oligomerization in expanded polyglutamine neurodegenerative disorders. Nature 421, 373-379.
77. Scherzinger, E., Lurz, R., Turmaine, M., Mangiarini, L., Hollenbach, B., Hasenbank, R., Bates, G. P., Davies, S. W., Lehrach, H., and Wanker, E. E. (1997). Huntingtin-encoded polyglutamine expansions form amyloid-like protein aggregates in vitro and in vivo. Cell 90, 549-558.
78. Schwikowski, B., Uetz, P., and Fields, S. (2000). A network of protein-protein interactions in yeast. Nature Biotechnol 17, 1030-1032.
79. Sittler, A., Walter, S.; Wedemeyer, N., Hasenbank, R., Scherzinger, E., Eickhoff, H., Bates, G., Lehrach, H., and Wanker, E. (1998). SH3GL3 associates with the Huntingtin exon 1 protein and promotes the formation of polygln-containing protein aggregates. Mol Cell 4, 427-436.
80. Turner, C., West, K., and Brown, M. (2001). Paxillin-ARF GAP signaling and the cytoskeleton. Curr Opin Cell Biol 13, 593-599.
81. Vitale, N., Patton, W. A., Moss, J., Vaughan, M., Lefkowitz, R. J., and Premont, R. T. (2000). GIT proteins, A novel family of phosphatidylinositol 3,4, 5-trisphosphate-stimulated GTPase-activating proteins for ARF6. J Biol Chem 275, 13901-13916.
82. von Mering, C., Krause, R., Snel, B., Cornell, M., Oliver, S. G., Fields, S., and Bork, P. (2002). Comparative assessment of large-scale data sets of protein-protein interactions. Nature 417, 399-403.
83. Wang, T., Kobayashi, T., Takimoto, R., Denes, A., Snyder, E., el-Deiry, W., and Brachmann, R. (2001). hADA3 is required for p53 activity. EMBO J. 20, 6404-6413.
84. Wanker, E., Rovira, C., Scherzinger, E., Hasenbank, R., Walter, S., Tait, D., Colicelli, J., and Lehrach, H. (1997). HIP-I: a huntingtin interacting protein isolated by the yeast two-hybrid system. Hum Mol Genet 3, 487-495.
85. Witke, W., Podtelejnikov, A., Di Nardo, A., Sutherland, J., Gurniak, C., Dotti, C., and Mann, M. (1998). In mouse brain profilin I and profilin 11 associate with regulators of the endocytic pathway and actin assembly. EMBO J. 17, 967-976.
86. Zhang, H., Webb, D. J., Asmussen, H., and Horwitz, A. F. (2003). Synapse formation is regulated by the signaling adaptor GIT1. J Cell Biol 161, 131-142.
87. Zhao, Z., Manser, E., Loo, T., and Lim, L. (2000). Coupling of PAK-interacting exchange factor PIX to GIT1 promotes focal complex disassembly. MolCell Biol 20, 6354-6363.

1. A method for generating a network of direct and indirect interaction partners of a disease-related (poly)peptide comprising the steps of
(a) contacting a selection of (poly)peptides suspected to contain one or several of said direct or indirect interaction partners with said disease-related (poly)peptides and optionally with known direct or indirect interaction partners of said disease-related (poly)peptide under conditions that allow the interaction between interaction partners to occur;
(b) detecting (poly)peptides that interact with said disease-related (poly)peptide or with said known direct or indirect interaction partners of said disease-related (poly)peptide;
(c) contacting (poly)peptides detected in step (b) with a selection of (poly)peptides suspected to contain one or several (poly)peptides interacting with said (poly)peptides detected in step (b) under conditions that allow the interaction between interaction partners to occur;
(d) detecting proteins that interact with said (poly)peptides detected in step (b);
(e) contacting said disease-related (poly)peptide and optionally said known direct or indirect interaction partners of said disease-related (poly)peptide, said (poly)peptides detected in steps (b) and (d) and a selection of proteins suspected to contain one or several (poly)peptides interacting with any of the afore mentioned (poly)peptides under conditions that allow the interaction between interaction partners to occur;
(f) detecting (poly)peptides that interact with said disease-related (poly)peptide and optionally said known direct or indirect interaction partners of said disease-related (poly)peptide or with said (poly)peptides identified in step (b) or (d); and
(g) generating a (poly)peptide-(poly)peptide interaction network of said disease-related (poly)peptide and optionally said known direct or indirect interaction partners of said disease-related (poly)peptide and said (poly)peptides identified in steps (b), (d) and (f).

2. The method of claim 1, wherein said contacting step (e) is effected in an interaction mating two hybrid approach.

3. The method of claim 1, said method comprising after step (d) and before step (e) the steps of:
(d′) contacting (poly)peptides detected in step (d) with a selection of (poly)peptides suspected to contain one or several (poly)peptides interacting with said (poly)peptides detected in step (d) under conditions that allow the interaction between interaction partners to occur; and
(d″) detecting proteins that interact with said (poly)peptides detected in step (d′).

4. The method of claim 3, wherein said disease-related protein is a protein suspected of being a causative agent of a hereditary disease.

5. The method of claim 4, wherein said disease-related protein is huntingtin and wherein said interaction partners are the interaction partners as shown in tables 6, 7 or 9.

6. The method of claim 5, said method comprising the step of determining the nucleotide sequence of a nucleic acid molecule encoding a direct or indirect interaction partner of the disease related protein.

7. The method of claim 6, wherein said selections of proteins are translated from a nucleic acid library.

8. The method of claim 7, wherein said selection of proteins in step (a) and/or (c) and/or (d′) and/or (e) is the same selection or a selection from the same source.

9. The method of claim 7, wherein said selection of proteins in step (a) and/or (c) and/or (d′) and/or (e) is a different selection or a selection from a different source.

10. The method of claim 9, wherein said method is performed by contacting the proteins on an array.

11. The method of claim 10, wherein said interactions are detected by using the yeast two-hybrid system.

12. The method of claim 11, containing after step (b), (d), (d″) or (f) the additional steps of isolating a nucleic acid molecule with homology to said cDNA expressing the encoded protein and testing it for its activity as a modulator of huntingtin, wherein said nucleic acid molecule is DNA, or RNA, preferably cDNA, or genomic or synthetic DNA or mRNA.

13-19. (canceled)

20. A (poly)peptide comprising an amino acid sequence of a protein listed in table 8.

21. The (poly)peptide of claim 20 fused to a heterologous (poly)peptide.

22. A protein complex comprising at least two proteins, wherein said at least two proteins are selected from the group of interaction partners listed in table 9.

23-24. (canceled)

25. A method of identifying whether a protein promotes huntingtin aggregation, comprising
(a) transfecting a first cell with a nucleic acid molecule encoding a variant of the huntingtin protein or a fragment thereof capable of forming huntingtin aggregates;
(b) co-transfecting a second cell with
(i.) a nucleic acid molecule encoding a variant of the huntingtin protein or a fragment thereof capable of forming huntingtin aggregates; and
(ii.) a nucleic acid molecule encoding a candidate modulator protein identified by the method of claim 1 or a nucleic acid molecule encoding a modulator protein selected from table 6 or table 7;
(c) expressing the proteins encoded by the transfected nucleic acid molecule of (a) and (b);
(d) isolating insoluble aggregates of huntingtin from the transfected cell of (a) and (b); and
(e) determining the amount of insoluble huntingtin aggregates from the transfected cell of (a) and (b)
wherein an increased amount of huntingtin aggregates isolated from the transfected cells of (b) in comparison with the amount of huntingtin aggregates isolated from the transfected cells of (a) is indicative of a protein's activity as an enhancer of huntingtin aggregation.

26. A method of identifying whether a protein inhibits huntingtin aggregation, comprising
(a) transfecting a first cell with a nucleic acid molecule encoding a variant of the huntingtin protein or a fragment thereof capable of forming huntingtin aggregates;
(b) co-transfecting a second cell with
(i.) a nucleic acid molecule encoding a variant of the huntingtin protein or a fragment thereof capable of forming huntingtin aggregates; and
(ii.) a nucleic acid molecule encoding a candidate modulator protein identified by the method of claim 1 or a nucleic acid molecule encoding a modulator protein selected from table 6 or table 7;
(c) expressing the proteins encoded by the transfected nucleic acid molecule of (a) and (b);
(d) isolating insoluble aggregates of huntingtin from the transfected cell of (a) and (b); and
(e) determining the amount of insoluble huntingtin aggregates from the transfected cell of (a) and (b)
wherein a reduced amount of huntingtin aggregates isolated from the transfected cells of (b) in comparison with the amount of huntingtin aggregates isolated from the transfected cells of (a) is indicative of a protein's activity as an inhibitor of huntingtin aggregation.

27. The method of claim 26, wherein prior to step (d) the cells are treated with an ionic detergent.

28. The method of claim 27, wherein the huntingtin aggregates are filtered or transferred onto a membrane.

29-31. (canceled)

32. A method of diagnosing Huntington's disease in a biological sample comprising the steps of
(a) contacting the sample with an antibody specific for a protein of table 6 or 7 or an antibody specific for the protein complex of claim 22; and
(b) detecting binding of the antibody to a protein complex, wherein the detection of binding is indicative of Huntington's disease or of a predisposition to develop Huntington's disease.

33-36. (canceled)
